(19) United States
(12) Patent Application Publication
(10) Pub. No.: US 2009/0263396 A1
(43) Pub. Date: Oct. 22, 2009

(54) INHIBITORS OF T-DARPP FOR USE IN COMBINATION ANTI-CANCER THERAPIES

(75) Inventors: Wael El-Rifai, Brentwood, TN (US); Abbes Belkhiri, Brentwood, TN (US)

Correspondence Address: FULBRIGHT & JAWORSKI L.L.P., 600 CONGRESS AVE., SUITE 2400, AUSTIN, TX, 78701, US

(73) Assignee: VANDERBILT UNIVERSITY

(21) Appl. No.: 12/272,288

(22) Filed: Nov. 17, 2008

Related U.S. Application Data
(60) Provisional application No. 60/988,131, filed on Nov. 15, 2007.

(51) Int. Cl. A61K 39/395; A61K 31/713; A61P 35/00

(52) U.S. Cl. 424/139.1; 514/44 A

(57) ABSTRACT

The present invention relates to an improved anti-HER2/Neu therapy comprising co-administration of an inhibitor of t-DARPP activity. In addition, inhibitors of t-DARPP can act as a monotherapy against cancers that express elevated t-DARPP activity, including elevated t-DARPP levels, or in combination with chemo- or radiotherapeutic anti-cancer treatments drugs.

This application claims benefit of U.S. Provisional Application Ser. No. 60/988,131, filed Nov. 15, 2007, the entire contents of which are hereby incorporated by reference.

This invention was made with government support under grant no. RO1 CA93999 awarded by the National Institutes of Health. The government has certain rights in the invention.

### BACKGROUND OF THE INVENTION

I. Field of the Invention

The present invention relates to the fields of oncology, genetics and molecular biology. More particular the invention relates to an improved cancer therapy utilizing anti-HER2/Neu in conjunction with an inhibitor of t-DARPP activity

II. Related Art

One of the molecular hallmarks of breast cancer is the amplification of the 17q12 region which includes the ERBB2 oncogene (Kauraniemi et al., 2003; Monni et al., 2001). Amplification of the HER2/Neu (ERBB2) gene occurs in approximately 25% of invasive breast cancers and is associated with poor patient outcome. This amplification is the most common mechanism for ERBB2 activation in breast cancer (Borg et al., 1989; Dandachi et al., 2004; Latta et al., 2002). The HER-2/neu oncogene, a member of the epidermal growth factor receptor family, encodes a transmembrane tyrosine kinase receptor that has been linked to prognosis and response to therapy with the anti-HER-2-humanized monoclonal antibody, trastuzumab (Herceptin; Genentech, South San Francisco, Calif.) in patients with advanced metastatic breast cancer (Arteaga et al. 2002; Ross et al., 2004). Inhibition of PI3K/Akt is required for the antitumor effect of HER2 inhibitors (Arteaga et al. 2002; Shin et al., 2002). HER2 overexpression, detected by IHC and/or FISH, is the biomarker predictive of good odds of response to treatment with the antibody. However, one of the major clinical problems encountered with trastuzumab treatment is that metastatic breast cancer patients, who initially responded to trastuzumab, demonstrated disease progression within one year of treatment initiation (Nahta et al., 2006). Preclinical studies have indicated that increased signaling via the PI3K/Akt pathway may contribute to trastuzumab resistance (Nahta et al., 2006).

Considerable interest has been shown in the 17q12 amplicon as one of the most frequently amplified regions in upper gastrointestinal adenocarcinomas and breast cancer (Kauraniemi et al., 2003; Varis et al., 2002). Analysis of this amplicon's structure revealed an approximate 280-kb common region of amplification that contains 10 transcribed sequences (Kauraniemi et al., 2003; Varis et al., 2002; Kauraniemi and Kallioniemi, 2006; Maqani et al., 2006). Cloning and physical mapping strategies of transcripts in this region have shown that DARPP-32 (Dopamine and cyclic AMP regulated phosphoprotein 32 kD) and its truncated variant (t-DARPP) are located next to ErbB2 within the same amplicon structure (Varis et al., 2002; Maqani et al., 2006; Varis et al., 2004). Interestingly, DARPP-32 and t-DARPP are frequently amplified and over-expressed in a number of adenocarcinomas that include breast, gastric, esophageal, colon, and prostate (El-Rifai et al., 2002; Beckler et al., 2003; Ebihara et al., 2004). Recent studies have shown that t-DARPP is a potent anti-apoptotic protein that counteracts drug-induced apoptosis (Belkhiri et al., 2005).

### SUMMARY OF THE INVENTION

Thus, in accordance with the present invention, there is provided a method of improving the effect of an anti-HER2/Neu therapy in a subject comprising administering to the subject an inhibitor of t-DARPP activity. The inhibitor of t-DARPP activity may inhibit t-DARPP mRNA synthesis or stability, or t-DARPP protein translation, such as an antisense nucleic acid, an siRNA, or an shRNA. Alternatively, the inhibitor of t-DARPP activity may inhibit the function of t-DARPP gene product, such as an antibody, a peptide or a small molecule that binds to t-DARPP. The anti-HER2/Neu therapy may be an anti-HER2/Neu antibody administration. The anti-HER2/Neu therapy may comprise a reduced dose as compared to an effective dose for the anti-HER2/Neu therapy provided in the absence of the inhibitor of t-DARPP activity. Either or both of the anti-HER2/Neu therapy and the inhibitor of t-DARPP activity may be administered to the subject more than once. The inhibitor of t-DARPP gene product function may be delivered by an expression vector encoding the inhibitor.

In another embodiment, there is provided a method of inhibiting trastuzumab resistance in a subject comprising administering to the subject an inhibitor of t-DARPP activity. The inhibitor of t-DARPP activity may inhibit t-DARPP mRNA synthesis or stability, or t-DARPP protein translation, such as an antisense nucleic acid, an siRNA, or an shRNA. Alternatively, the inhibitor of t-DARPP activity may inhibit the function of t-DARPP gene product, such as an antibody, a peptide or a small molecule that binds to t-DARPP. The inhibitor of t-DARPP gene product function may be delivered by an expression vector encoding the inhibitor.

In yet another embodiment, there is provided a method of inducing apoptosis in a cancer cell that overexpresses ERBB2 relative to a non-cancer cell comprising contacting the cell with an anti-HER2/Neu agent and an inhibitor of t-DARPP activity. The inhibitor of t-DARPP activity may inhibit t-DARPP mRNA synthesis or stability, or t-DARPP protein translation, such as an antisense nucleic acid, an siRNA, or an shRNA. Alternatively, the inhibitor of t-DARPP activity may inhibit the function of t-DARPP gene product, such as an antibody, a peptide or a small molecule that binds to t-DARPP. The inhibitor of t-DARPP gene product function may be delivered by an expression vector encoding the inhibitor. The anti-HER2/Neu therapy may be an anti-HER2/Neu antibody administration. The anti-HER2/Neu therapy may comprise a reduced dose as compared to an effective dose for the anti-HER2/Neu therapy provided in the absence of the inhibitor of t-DARPP activity. Either or both of the anti-HER2/Neu therapy and the inhibitor of t-DARPP activity may be administered to the subject more than once.

In still yet another embodiment, there is provided a method of treating cancer in a subject comprising administering to the subject an anti-HER2/Neu therapy and an inhibitor of t-DARPP activity. The inhibitor of t-DARPP activity may be administered prior to, during or after the HER2-Neu therapy. The cancer may be breast cancer, such as a Herceptin-resistant breast cancer or a recurrent cancer. The inhibitor of t-DARPP activity may inhibit t-DARPP mRNA synthesis or stability, or t-DARPP protein translation, such as an antisense nucleic acid, an siRNA, or an shRNA. Alternatively, the inhibitor of t-DARPP activity may inhibit the function of t-DARPP gene product, such as an antibody, a peptide or a small molecule that binds to t-DARPP. The anti-HER2/Neu therapy may be an anti-HER2/Neu antibody administration. The method may further comprise altering a dose of either anti-HER2/Neu therapy or the inhibitor of t-DARPP activity based on a clinical response of the patient. The method may further comprise assessing t-DARPP protein level or t-DARPP gene amplification in cancer cells of the subject. The inhibitor of t-DARPP gene product function may be delivered by an expression vector encoding the inhibitor. Either or both of the anti-HER2/Neu therapy and the inhibitor of t-DARPP activity may be administered to the subject more than once.

In yet a further embodiment, there is provided a method of inhibiting a cancer cell in a subject, wherein the cancer cell expresses elevated t-DARPP activity as compared to a comparable non-cancer cell, comprising administering to the subject an inhibitor of t-DARPP activity. The inhibitor of t-DARPP activity may inhibit t-DARPP mRNA synthesis or stability, or t-DARPP protein translation. The inhibitor may be an antisense nucleic acid, an siRNA, or an shRNA. The inhibitor of t-DARPP activity may inhibit t-DARPP gene product function, such as an antibody, a peptide or a small molecule that binds to t-DARPP. The inhibitor of t-DARPP activity may be administered to the subject more than once. The inhibitor of t-DARPP gene product function may be delivered by an expression vector encoding the inhibitor. The cancer cell may be a gastric carcinoma cell, an esophageal carcinoma cell, or a breast cancer cell.

It is contemplated that any method or composition described herein can be implemented with respect to any other method or composition described herein.

The use of the word “a” or “an” when used in conjunction with the term “comprising” in the claims and/or the specification may mean “one,” but it is also consistent with the meaning of “one or more,” “at least one,” and “one or more than one.”

These, and other, embodiments of the invention will be better appreciated and understood when considered in conjunction with the following description and the accompanying drawings. It should be understood, however, that the following description, while indicating various embodiments of the invention and numerous specific details thereof, is given by way of illustration and not of limitation. Many substitutions, modifications, additions and/or rearrangements may be made within the scope of the invention without departing from the spirit thereof, and the invention includes all such substitutions, modifications, additions and/or rearrangements.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein:

FIGS. 1A-D—t-DARPP overexpression correlates with trastuzumab resistance and increased cell survival. Cells were treated with vehicle or trastuzumab (5 and 10 μg/ml) for 24 h and 48 h and then subjected to CellTiter-Glo Luminescent Cell Viability Assay. (FIG. 1A) Cell survival was significantly lower in the parental BT-474 cell line as compared to trastuzumab resistant HR5 and HR6 cells for all tested trastuzumab concentrations and time points. (FIG. 1B) Protein extracts from non-treated cells (BT-474, HR5, and HR6) were subjected to Western blot analysis of ERBB2 and t-DARPP. ERBB2 protein levels were comparable in all cells. However, t-DARPP protein levels were dramatically higher in HR5 and HR6 as compared to BT-474 cells. (FIG. 1C) similar to trastuzumab resistant cells HR5 and HR6 (panel A), cell survival of SKBR-3 cells stably expressing t-DARPP (Clones #1 and #7) was also markedly higher than control SKBR-3 cells following treatment with trastuzumab. (FIG. 1D) Protein extracts from non-treated SKBR-3-t-DARPP-1, SKBR-3-t-DARPP-7, and control SKBR-3 cells were subjected to immunobloting of EBB2 and t-DARPP. As expected, t-DARPP protein levels were significantly higher in SKBR-3-t-DARPP-1 and SKBR-3-t-DARPP-7 clones as compared to control SKBR-3 cell line. ERBB2 protein levels were comparable in all tested cells. Gel loading was normalized for equal β-actin.

FIGS. 2A-C—Transcriptional upregulation of t-DARPP in trastuzumab-resistant cell lines is not associated with gene amplification of t-DARPP and ERBB2 or with altered ERBB2 expression. Gene-specific primers for DNA and mRNA specific sequences of t-DARPP, ERBB2, β-ACTIN and HPRT1 were used for PCR and real-time PCR in BT474, HR5, and HR6 cells, and the results were normalized to β-ACTIN and HPRT1. (FIG. 2A) t-DARPP mRNA expression levels in HR5 and HR6 cells were 25- to 100-fold higher as compared to BT-474 cells (asterisks indicate P≦0.001). In contrast, t-DARPP gene amplification was not affected in all cells. (FIG. 2B) ERBB2 mRNA expression and gene amplification levels remained unchanged in all cells. (FIG. 2C) DNA bisulfite treatment and pyrosequencing analysis of DNA methylation. DNA from BT-474, HR5, and HR6 cells was extracted and modified by bisulfite treatment as described in Materials and Methods. A CpG island from −1438 to −830 of t-DARPP was amplified with PCR using specific primers. The PCR products were then subjected to pyrosequencing analysis to determine DNA methylation of 10 CpG sites within −1161 to −1109 region of t-DARPP. Comparable levels of DNA methylation were detected in all three cell lines, indicating that DNA methylation of t-DARPP CpG island does not regulate its expression in these cell lines.

FIGS. 3A-D—Knockdown of t-DARPP induces apoptosis in trastuzumab-resistant and -sensitive breast cancer cells. (FIG. 3A) BT-474, HR5, and HR6 cells were transfected with control scrambled siRNA or t-DARPP siRNA oligonucleotides and then subjected to Titer-Glo-Luminescent Viability Assay 24 hr and 48 hr post-transfection. Overall, cell survival was significantly lower in all cells transfected with t-DARPP siRNA as compared to control cells. In addition, cell survival of HR5 and HR6 is dramatically lower after knockdown of t-DARPP (P≦0.01). (FIGS. 3B-D) Cells were transfected with control scrambled siRNA or t-DARPP siRNA oligonucleotides. The cells were then treated, 24 hr post-transfection, with 20 μg/ml of trastuzumab or vehicle (control) for 48 h. (FIG. 3B) Protein extracts from BT-23474 cells (trastuzumabsenstive) were subjected to Western blot analysis of t-DARPP, ERBB2 and caspase 3. As expected, trastuzumab alone down-regulated ERBB2 protein level and induced apoptosis as indicated by appearance of cleaved caspase 3 fragment (lane 2). Knockdown of t-DARPP alone (lane 3) and in combination with trastuzumab (lane 4) significantly downregulated ERBB2 and activated caspase 3. (FIG. 3C) Protein extracts from HR5 cells (trastuzumab-resistant) were subjected to immunoblotting of t-DARPP, ERBB2 and caspase 3. As expected, trastuzumab alone neither downregulated ERBB2 protein level nor activated caspase 3 (lane 2). Knockdown of t-DARPP alone (lane 3) and in combination with trastuzumab (lane 4) down-regulated ERBB2 and activated caspase 3. (FIG. 3D) Protein extracts from HR6 cells (trastuzumab-resistant) were subjected to Western blot analysis of t-DARPP, ERBB2 and caspase 3. As in HR5 cells (FIG. 3B), trastuzumab alone did not downregulate ERBB2 or activate caspase 3 (lane 2). In contrast, knockdown of t-DARPP alone (lane 3) or in combination with trastuzumab (lane 4) markedly down-regulated ERBB2 and activated caspase 3. Gel loading was normalized for equal β-ACTIN.

FIG. 4—Knockdown of t-DARPP downregulates ERBB2 protein and inhibits the PI3K/Akt survival pathway in trastuzumab-resistant cells. Protein extracts from HR6 cells transfected with control scrambled siRNA or t-DARPP siRNA oligonucleotides were subjected to Western blot analysis of t-DARPP, ERBB2, HSP90, pAKT(S473), AKT, BCL2, and caspase 3. Knockdown of t-DARPP did not affect HSP90 protein level, but dramatically decreased ERBB2 and Bcl2 protein levels and phosphorylated AKT, and resulted in marked activation of caspase 3. Gel loading was normalized for equal β-Actin.

FIG. 5—t-DARPP induces transformation of primary MEFs. Retroviral construct expressing either t-DARPP or E1A, which is a viral oncogene incapable of transforming primary cells alone, was used to immortalize primary MEF cells (left panel). As shown on the right panel, a co-operation of t-DARPP and E1A was shown to be sufficient to transform primary MEFs.

FIGS. 6A-C—t-DARPP transforms NIH3T3 cells. (FIG. 6A) NIH3T3 cells were retrovirally transduced with empty vector, or t-DARPP. Three×10⁵ cells were seeded per 10 cm culture dish (4 dishes per group). After 3 weeks, the cells were fixed with ice-cold methanol for 10 minutes then stained with 0.5% Coomassie blue R-250. (FIG. 6B) Western blot confirmed the expression of the intended proteins. (FIG. 6C) The frequency of focus formation was determined by counting individual foci (≧1 mm) in four separate dishes for each group.

FIGS. 7A-C—t-DARPP induces tumor formation in mice. (FIG. 7A) representative mouse for NIH3T3-t-DARPP xenografting is shown and displays a large tumor formation (1000 mm³) at the site of injection and a distant metastatic tumor, ˜two inches away, was also seen as indicated by arrows. (FIG. 7B) The H&E section demonstrated a tumor that was classified as a high-grade sarcoma. (FIG. 7C) IHC analysis with a c-terminal DARPP-32 antibody that detects t-DARPP confirmed the expression of t-DARPP in tumor cells.

FIG. 8A-D—t-DARPP induces AKT kinase activity. (FIG. 8A) Protein extracts (15 μg) from AGS cells expressing t-DARPP or empty vector and purified active AKT (6 ng) as a control were subjected to an in vitro AKT kinase activity assay analysis. The relative AKT kinase activity in t-DARPP-expressing cells was induced with more than two-fold compared with control cells. (FIG. 8B) Phosphorylation of AKT was higher in t-DARPP-expressing cells than control cells. This directly correlated with GSK3β phosphorylation and with both BCL-2 and t-DARPP protein levels. GSK3β is a direct target of AKT and its phosphorylation is a measure of AKT activity. (FIG. 8C) Protein extracts from AGS cells expressing t-DARPP or empty vector and transfected with t-DARPP siRNA or control siRNA were subjected to Western blot analysis of p-^{Ser473} AKT, AKT, and t-DARPP. Knockdown of t-DARPP in cells expressing t-DARPP (lane 4) decreased AKT phosphorylation to a comparable level as control cells (lane 3). (FIG. 8D) Tetracycline-inducible AGS cells expressing t-DARPP were treated with doxycycline for 24 hr to induce expression and then removed after various time points as indicated in the figure.

FIGS. 9A-B—The knockdown of t-DARPP reverses its effects on pAKT and BCL2 protein levels. (FIG. 9A) AGS cells stably expressing empty vector or t-DARPP demonstrated a significant up-regulation of pAKT and BCL-2 levels (≧10 fold). (FIG. 9B) For validation, knockdown of t-DARPP in four cell lines (two gastric; MKN45 and SNU1, and two breast cancer; HR6 and HCC1569) with constitutive expression of t-DARPP. The knockdown of t-DARPP led to significant reductions in pAKT and BCL-2 protein levels in all four cell lines. These results confirm that AKT and BCL-2 levels are regulated by t-DARPP.

FIGS. 10A-B—Inhibition of tumor growth in t-DARPP expressing cancer cells in a murine cancer model. The human cancer cell line SNU16 has high levels of endogenous t-DARPP. Sixteen randomized mice were xenografted with SNU16 cells stably expressing the tetracycline-inducible sh-t-DARPP (8) or the empty vector (8). All mice received doxycycline (2 mg/ml) in drinking water, and the mice were monitored for 30 days.

### DETAILED DESCRIPTION OF THE PREFERRED EMBODIMENTS

### I. The Present Invention

BT-474 and SKBR-3 human breast cancer cells are known to display DNA amplification and mRNA and protein over-expression of ERBB2/HER2 (Alimandi et al., 1995; Schwartze, 1987). These cells have been shown to be HER2-dependent where the inhibition of the HER2 leads to growth arrest and/or tumor cell death both in vitro and in vivo (Baselga et al., 1998; Sliwkowski et al., 1999). Therefore, these cells provided a unique opportunity to determine if t-DARPP expression leads to trastuzumab resistance. The HR5 and HR6 trastuzumab-resistant cells were recently developed from their progenitor BT-474 through continuous passaging in vivo (Ritter et al., 2007). Interestingly, the inventors detected t-DARPP overexpression in HR5 and HR6 cells as compared to BT-474 progenitor cells. Interestingly, the DNA amplification levels of t-DARPP remained unaffected. One of the main epigenetic events involved in transcription regulation is the change in promoter DNA methylation levels. The overexpression of several oncogenes has been reported to be controlled by hypomethylation of their respective promoters. The inventors' quantitative analysis of the CpG island in t-DARPP promoter indicated no changes in the methylation levels between BT-474 cells and their HR5 and HR6 cells that express high levels of t-DARPP transcript and protein. Taken together, these findings suggest that t-DARPP regulation in these cells is independent on their DNA amplification or promoter methylation levels. The transcription factors that may regulate t-DARPP remain unknown and future analysis in this direction could reveal the molecular mechanism underlying t-DARPP transcription regulation.

The inventors have shown previously that t-DARPP overexpression mediates cell survival in epithelial cells (Belkhiri et al., 2005; Belkhiri et al., 2008a). Therefore, the inventors investigated if t-DARPP expression mediates the trastuzumab resistance phenotype. Indeed the HR5, HR6 and SKBR3 cells stably expressing t-DARPP showed increased cell survival following treatment with trastuzumab as compared to their controls. This finding highlighted the possibility that t-DARPP expression could be one of the molecular events that contribute to trastuzumab resistance. The BT-474 and SKBR-3 cells are well known as being sensitive to trastuzumab treatment with an IC₅₀ of ˜0.2-0.5 μg/ml (Yakes et al., 2002) and the inventors' findings were confirmed even when treating cells with high doses of trastuzumab (5, 10, and 20 μg/ml). Several mechanisms of trastuzumab action have been reported (reviewed by Nahta et al., 2006; Nahta et al., 2005).

Trastuzumab suppresses ERBB2-mediated activation of the PI3K and MAPK signaling pathways, and this may be through internalization and degradation of the ERBB2 receptor (Sliwkowski et al., 1999; Baselga and Albanell, 2001) or as a result of disrupting the interaction between ERBB2 and the Src tyrosine kinase, which leads to activation of the PI3K inhibitor PTEN (Nagata et al., 2004). Multiple mechanisms by which ERBB2-overexpressing tumors escape trastuzumab-mediated cytotoxicity have been proposed. One of which is failure of trastuzumab to inhibit the ERBB2-mediated signaling pathways (Stephens et al., 2004) such as the Pi3K/AKT pathway which is believed to play a major role in Herceptin resistance. The inventors' findings demonstrate that knockdown of endogenous t-DARP levels led to a significant reduction in cell survival at several time points. Interestingly, this effect was associated with a remarkable abrogation of ERBB2 levels and induction of cleaved caspase 3 indicating progression of cells towards apoptosis. Therefore, t-DARPP may contribute to the Trastuzumab resistance by blocking the trastuzumab effect on ErBB2 and maintaining its high levels in these cells despite the treatment. Several mechanisms may be involved in stabilization of ERBB2 levels by t-DARPP which requires further investigations.

The inventors have also shown that t-DARPP knockdown led to a reduction in pAKT (Ser 473) and BCL2 levels. These effects reflect and confirm that role of t-DARPP in cell survival. AKT is a major survival pathway which has been implicated in Herceptin resistance (Nagata et al., 2004; Chan et al., 2005). Consistent with this finding, the inventors observed an increase in BCL2 protein levels. The BCL2 family proteins are pivotal regulators of apoptotic cell death that counteracts drug-induced apoptosis and shifts the balance towards cancer-cell survival through stabilization of the mitochondrial transmembrane potential and inhibition of release of cytochrome c and activation of caspases (Yang et al., 1997; Kluck et al., 1997; Cory and Adams, 2002). The inventors have recently shown that t-DARPP expression leads to increased kinase activity of AKT and leads to upregulation of BCL2 through Creb/Atf dependent mechanism that require Akt in gastric cells. Taken together, the inventors' findings underscore an important role of t-DARPP in trastuzumab resistance.

Thus, the inventors now describe studies of trastuzumab-resistance in BT-474 and its in vivo derivatives HR5 and 6 (Herceptin resistant). They demonstrate that although resistant cells and their progenitor BT-474 co-amplify HER2 and t-DARPP genes, the resistant cells further overexpress t-DARPP to higher levels than BT-474. The knockdown of t-DARPP was sufficient to promote apoptosis in BT-474 and HR cells thus overcoming the trastuzumab-resistance phenotype in HR cells. These results point to t-DARPP as a novel therapeutic target in patients with the HER2 amplicon. In addition, in vivo animal data shows that anti-t-DARPP therapies as described herein can function as a monotherapy against cancers with elevated t-DARPP activity. These and other aspects of the invention are described in detail below.

### II. t-DARPP

DARPP-32 stands for dopamine- and cyclic AMP-regulated phosphoprotein with molecular weight 32 kDa. DARPP-32 (submitted to the GenBank/EMBL database with accession no. AF464196) is also the name of the gene that creates the protein. DARPP32 is related to dopamine, glutamate and adenosine; and may be related to schizophrenia, Parkinson's disease or EPS (extra-pyramidal symptoms). A considerable proportion of the psychomotor effects of cannabinoids can be accounted for by a signaling cascade in striatal projection neurons involving PKA-dependent phosphorylation of DARPP-32, achieved via modulation of dopamine D2 and adenosine A2A transmission.

Currently there is strong evidence that DARPP-32 may be the shared chain in the action of multiple drugs including cocaine, amphetamine, nicotine, caffeine, LSD, PCP, ethanol and morphine. A common version of the DARPP-32 gene has been shown to improve the transfer of information between the striatum and the prefrontal cortex. When this process works well it leads to improved and more flexible cognition. However this same version of the gene has been linked to an increased risk of schizophrenia. While this gene typically improves cognitive ability, it may have a negative effect when other genetic and environmental factors interfere.

The inventors previously discovered a novel truncated isoform of DARPP-32, designated t-DARPP (GenBank accession no. AY070271), which is overexpressed in gastric cancers. Using quantitative real-time reverse transcription-PCR, Western blots, and staining of tumor tissue arrays, the two DARPP mRNA transcripts and proteins were found to be overexpressed in gastric cancer cells and exhibited abundant protein overexpression in neoplastic but not normal gastric epithelial cells. DARPP-32 is the only known protein that acts as a protein phosphatase 1 inhibitor or a protein kinase A inhibitor. The truncated isoform, t-DARPP, lacks the phosphorylation site related to protein phosphatase 1 inhibition but maintains the phosphorylation site with the protein kinase A inhibitory effect. These results were the first to indicate the presence of these signaling molecules in human cancer and suggest that they may be important for gastric tumorigenesis (E1-Rifai et al., 2002).

A. Features of the Polypeptide

The t-DARPP gene utilizes an alternative first exon located within intron 1 of DARPP-32 to form the first exon for the truncated molecule. Thus, DARPP-32 encodes a protein of 204 amino acids, whereas t-DARPP encodes a 168-amino acid protein. DARPP-32 contains four phosphorylation sites at Thr³⁴, Thr⁷⁵, Ser¹⁰², and Ser¹³⁷, whereas t-DARPP lacks the Thr³⁴ phosphorylation site of DARPP-32, which is located in the omitted first exon.

B. Peptides

The present invention contemplates the use of peptides and fragments of t-DARPP for generation of antibodies and for use as therapeutic compositions in the treatment of hyper-inflammatory disorders. t-DARPP peptides will comprise molecules of 6 to about 100 residues in length. A particular length may be less than 50 residues, less than 40 residues, less than 35 residues, less than 30 residues, less than 25 residues, less than 20 residues, less than 15 residues, including 6, 7, 8, 9, 10, 11, 12, 13, 14, 15, 16, 17, 18, 19 or 20 residues. The peptides may be generated synthetically or by recombinant techniques, and are purified according to known methods, such as precipitation (e.g., ammonium sulfate), HPLC, ion exchange chromatography, affinity chromatography (including immunoaffinity chromatography) or various size separations (sedimentation, gel electrophoresis, gel filtration).

The peptides may be labeled using various molecules, such as fluorescent, chromogenic or colorimetric agents. The peptides may also be linked to other molecules, including other anti-inflammatory agents. The links may be direct or through distinct linker molecules. The linker molecules in turn may be subject, in vivo, to cleavage, thereby releasing the agent from the peptide. Peptides may also be rendered multimeric by linking to larger, and possibly inert, carrier molecules.

C. Analogs and Mimetics

It also is contemplated in the present invention that variants or analogs of t-DARPP peptides may block t-DARPP activity, and hence reduce its impact on Her-2/Neu therapy. Sequence variants of t-DARPP peptides, primarily making conservative amino acid substitutions, may provide improved compositions. Substitutional variants typically contain the exchange of one amino acid for another at one or more sites within the protein, and may be designed to modulate one or more properties of the polypeptide, such as stability against proteolytic cleavage, without the loss of other functions or properties. Substitutions of this kind preferably are conservative, that is, one amino acid is replaced with one of similar shape and charge. Conservative substitutions are well known in the art and include, for example, the changes of: alanine to serine; arginine to lysine; asparagine to glutamine or histidine; aspartate to glutamate; cysteine to serine; glutamine to asparagine; glutamate to aspartate; glycine to proline; histidine to asparagine or glutamine; isoleucine to leucine or valine; leucine to valine or isoleucine; lysine to arginine; methionine to leucine or isoleucine; phenylalanine to tyrosine, leucine or methionine; serine to threonine; threonine to serine; tryptophan to tyrosine; tyrosine to tryptophan or phenylalanine; and valine to isoleucine or leucine.

The following is a discussion based upon changing of the amino acids of a peptide to create an equivalent, or even an improved, second-generation molecule. For example, certain amino acids may be substituted for other amino acids in a protein structure without appreciable loss of interactive binding capacity with structures such as, for example, antigen-binding regions of antibodies or binding sites on substrate molecules. Since it is the interactive capacity and nature of a peptide that defines that peptide's biological functional activity, certain amino acid substitutions can be made in a protein sequence, and its underlying DNA coding sequence, and nevertheless obtain a peptide with like properties. It is thus contemplated by the inventors that various changes may be made in the DNA sequences coding the peptide without appreciable loss of their biological utility or activity, as discussed below.

In making such changes, the hydropathic index of amino acids may be considered. The importance of the hydropathic amino acid index in conferring interactive biologic function on a protein is generally understood in the art (Kyte and Doolittle, 1982). It is accepted that the relative hydropathic character of the amino acid contributes to the secondary structure of the resultant peptide, which in turn defines the interaction of the peptide with other molecules.

Each amino acid has been assigned a hydropathic index on the basis of their hydrophobicity and charge characteristics (Kyte and Doolittle, 1982), these are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

It is known in the art that certain amino acids may be substituted by other amino acids having a similar hydropathic index or score and still result in a peptide with similar biological activity, i.e., still obtain a biological functionally equivalent protein. In making such changes, the substitution of amino acids whose hydropathic indices are within ±2 is preferred, those which are within ±1 are particularly preferred, and those within ±0.5 are even more particularly preferred.

It is also understood in the art that the substitution of like amino acids can be made effectively on the basis of hydrophilicity. U.S. Pat. No. 4,554,101, incorporated herein by reference, states that the greatest local average hydrophilicity of a protein, as governed by the hydrophilicity of its adjacent amino acids, correlates with a biological property of the protein. As detailed in U.S. Pat. No. 4,554,101, the following hydrophilicity values have been assigned to amino acid residues: arginine (+3.0); lysine (+3.0); aspartate (+3.0±1); glutamate (+3.0±1); serine (+0.3); asparagine (+0.2); glutamine (+0.2); glycine (0); threonine (−0.4); proline (−0.5±1); alanine (−0.5); histidine *-0.5); cysteine (−1.0); methionine (−1.3); valine (−1.5); leucine (−1.8); isoleucine (−1.8); tyrosine (−2.3); phenylalanine (−2.5); tryptophan (−3.4).

It is understood that an amino acid can be substituted for another having a similar hydrophilicity value and still obtain a biologically equivalent and immunologically equivalent protein. In such changes, the substitution of amino acids whose hydrophilicity values are within ±2 is preferred, those that are within ±1 are particularly preferred, and those within ±0.5 are even more particularly preferred.

As outlined above, amino acid substitutions are generally based on the relative similarity of the amino acid side-chain substituents, for example, their hydrophobicity, hydrophilicity, charge, size, and the like. Exemplary substitutions that take various of the foregoing characteristics into consideration are well known to those of skill in the art and include: arginine and lysine; glutamate and aspartate; serine and threonine; glutamine and asparagine; and valine, leucine and isoleucine.

Another embodiment for the preparation of polypeptides according to the invention is the use of peptide mimetics. Mimetics are peptide containing molecules that mimic elements of protein secondary structure (Johnson et al, 1993). The underlying rationale behind the use of peptide mimetics is that the peptide backbone of proteins exists chiefly to orient amino acid side chains in such a way as to facilitate molecular interactions, such as those of antibody and antigen. A peptide mimetic is expected to permit molecular interactions similar to the natural molecule.

The present invention also may employ peptides that comprise modified, non-natural and/or unusual amino acids. Table 1 provides exemplary, but not limiting, modified, non-natural and/or unusual amino acids are provided herein below. Chemical synthesis may be employed to incorporate such amino acids into the peptides of interest.

TABLE 1
Modified, Non-Natural and Unusual Amino Acids

| Abbr. | Amino Acid |
|---|---|
| Aad | 2-Aminoadipic acid |
| BAad | 3-Aminoadipic acid |
| BAla | beta-alanine, beta-Amino-propionic acid |
| Abu | 2-Aminobutyric acid |
| 4Abu | 4-Aminobutyric acid, piperidinic acid |
| Acp | 6-Aminocaproic acid |
| Ahe | 2-Aminoheptanoic acid |
| Aib | 2-Aminoisobutyric acid |
| BAib | 3-Aminoisobutyric acid |
| Apm | 2-Aminopimelic acid |
| Dbu | 2,4-Diaminobutyric acid |
| Des | Desmosine |
| Dpm | 2,2′-Diaminopimelic acid |
| Dpr | 2,3-Diaminopropionic acid |
| EtGly | N-Ethylglycine |
| EtAsn | N-Ethylasparagine |
| Hyl | Hydroxylysine |
| AHyl | allo-Hydroxylysine |
| 3Hyp | 3-Hydroxyproline |
| 4Hyp | 4-Hydroxyproline |
| Ide | Isodesmosine |
| Aile | allo-Isoleucine |
| MeGly | N-Methylglycine, sarcosine |
| MeIle | N-Methylisoleucine |
| MeLys | 6-N-Methyllysine |
| MeVal | N-Methylvaline |
| Nva | Norvaline |
| Nle | Norleucine |
| Orn | Ornithine |

In addition to the variants discussed above, the present inventors also contemplate that structurally similar compounds may be formulated to mimic the key portions of peptide or polypeptides of the present invention. Such compounds, which may be termed peptidomimetics, may be used in the same manner as the peptides of the invention and, hence, also are functional equivalents.

Certain mimetics that mimic elements of protein secondary and tertiary structure are described in Johnson et al. (1993). The underlying rationale behind the use of peptide mimetics is that the peptide backbone of proteins exists chiefly to orient amino acid side chains in such a way as to facilitate molecular interactions, such as those of antibody and/or antigen. A peptide mimetic is thus designed to permit molecular interactions similar to the natural molecule.

Some successful applications of the peptide mimetic concept have focused on mimetics of β-turns within proteins, which are known to be highly antigenic. Likely β-turn structure within a polypeptide can be predicted by computer-based algorithms, as discussed herein. Once the component amino acids of the turn are determined, mimetics can be constructed to achieve a similar spatial orientation of the essential elements of the amino acid side chains.

Beta II turns have been mimicked successfully using cyclic L-pentapeptides and those with D-amino acids (Weisshoff et al., 1999). Also, Johannesson et al. (1999) report on bicyclic tripeptides with reverse turn inducing properties.

Methods for generating specific structures have been disclosed in the art. For example, alpha-helix mimetics are disclosed in U.S. Pat. Nos. 5,446,128; 5,710,245; 5,840,833; and 5,859,184. Theses structures render the peptide or protein more thermally stable, also increase resistance to proteolytic degradation. Six, seven, eleven, twelve, thirteen and fourteen membered ring structures are disclosed.

Methods for generating conformationally-restricted β turns and β bulges are described, for example, in U.S. Pat. Nos. 5,440,013; 5,618,914; and 5,670,155. β-turns permit changed side substituents without having changes in corresponding backbone conformation, and have appropriate termini for incorporation into peptides by standard synthesis procedures. Other types of mimetic turns include reverse and γ turns. Reverse turn mimetics are disclosed in U.S. Pat. Nos. 5,475,085 and 5,929,237, and γ turn mimetics are described in U.S. Pat. Nos. 5,672,681 and 5,674,976.

D. Fusions

Another variant is a fusion. This molecule generally has all or a substantial portion of the original molecule, in this case a peptide comprising a t-Darpp sequence, linked at the N- or C-terminus to all or a portion of a second peptide or polypeptide. For example, fusions may employ leader sequences from other species to permit the recombinant expression of a protein in a heterologous host. Another useful fusion includes the addition of a immunologically active domain, such as an antibody epitope, to facilitate purification of the fusion protein. Inclusion of a cleavage site at or near the fusion junction will facilitate removal of the extraneous polypeptide after purification. Other useful fusions include linking of functional domains, such as active sites from enzymes, glycosylation domains, cellular targeting signals or transmembrane regions.

E. Purification of Proteins

It will be desirable to purify t-DARPP, fragments, peptides or variants thereof. Protein purification techniques are well known to those of skill in the art. These techniques involve, at one level, the crude fractionation of the cellular milieu to polypeptide and non-polypeptide fractions. Having separated the polypeptide from other proteins, the polypeptide of interest may be further purified using chromatographic and electrophoretic techniques to achieve partial or complete purification (or purification to homogeneity). Analytical methods particularly suited to the preparation of a pure peptide are ion-exchange chromatography, exclusion chromatography; polyacrylamide gel electrophoresis; isoelectric focusing. A particularly efficient method of purifying peptides is fast protein liquid chromatography or even HPLC.

Certain aspects of the present invention concern the purification, and in particular embodiments, the substantial purification, of an encoded protein or peptide. The term “purified protein or peptide” as used herein, is intended to refer to a composition, isolatable from other components, wherein the protein or peptide is purified to any degree relative to its naturally-obtainable state. A purified protein or peptide therefore also refers to a protein or peptide, free from the environment in which it may naturally occur.

Generally, “purified” will refer to a protein or peptide composition that has been subjected to fractionation to remove various other components, and which composition substantially retains its expressed biological activity. Where the term “substantially purified” is used, this designation will refer to a composition in which the protein or peptide forms the major component of the composition, such as constituting about 50%, about 60%, about 70%, about 80%, about 90%, about 95% or more of the proteins in the composition.

Various methods for quantifying the degree of purification of the protein or peptide will be known to those of skill in the art in light of the present disclosure. These include, for example, determining the specific activity of an active fraction, or assessing the amount of polypeptides within a fraction by SDS/PAGE analysis. A preferred method for assessing the purity of a fraction is to calculate the specific activity of the fraction, to compare it to the specific activity of the initial extract, and to thus calculate the degree of purity, herein assessed by a “-fold purification number.” The actual units used to represent the amount of activity will, of course, be dependent upon the particular assay technique chosen to follow the purification and whether or not the expressed protein or peptide exhibits a detectable activity.

Various techniques suitable for use in protein purification will be well known to those of skill in the art. These include, for example, precipitation with ammonium sulphate, PEG, antibodies and the like or by heat denaturation, followed by centrifugation; chromatography steps such as ion exchange, gel filtration, reverse phase, hydroxylapatite and affinity chromatography; isoelectric focusing; gel electrophoresis; and combinations of such and other techniques. As is generally known in the art, it is believed that the order of conducting the various purification steps may be changed, or that certain steps may be omitted, and still result in a suitable method for the preparation of a substantially purified protein or peptide.

There is no general requirement that the protein or peptide always be provided in their most purified state. Indeed, it is contemplated that less substantially purified products will have utility in certain embodiments. Partial purification may be accomplished by using fewer purification steps in combination, or by utilizing different forms of the same general purification scheme. For example, it is appreciated that a cation-exchange column chromatography performed utilizing an HPLC apparatus will generally result in a greater “-fold” purification than the same technique utilizing a low pressure chromatography system. Methods exhibiting a lower degree of relative purification may have advantages in total recovery of protein product, or in maintaining the activity of an expressed protein.

It is known that the migration of a polypeptide can vary, sometimes significantly, with different conditions of SDS/PAGE (Capaldi et al., 1977). It will therefore be appreciated that under differing electrophoresis conditions, the apparent molecular weights of purified or partially purified expression products may vary.

High Performance Liquid Chromatography (HPLC) is characterized by a very rapid separation with extraordinary resolution of peaks. This is achieved by the use of very fine particles and high pressure to maintain an adequate flow rate. Separation can be accomplished in a matter of minutes, or at most an hour. Moreover, only a very small volume of the sample is needed because the particles are so small and close-packed that the void volume is a very small fraction of the bed volume. Also, the concentration of the sample need not be very great because the bands are so narrow that there is very little dilution of the sample.

Gel chromatography, or molecular sieve chromatography, is a special type of partition chromatography that is based on molecular size. The theory behind gel chromatography is that the column, which is prepared with tiny particles of an inert substance that contain small pores, separates larger molecules from smaller molecules as they pass through or around the pores, depending on their size. As long as the material of which the particles are made does not adsorb the molecules, the sole factor determining rate of flow is the size. Hence, molecules are eluted from the column in decreasing size, so long as the shape is relatively constant. Gel chromatography is unsurpassed for separating molecules of different size because separation is independent of all other factors such as pH, ionic strength, temperature, etc. There also is virtually no adsorption, less zone spreading and the elution volume is related in a simple matter to molecular weight.

Affinity Chromatography is a chromatographic procedure that relies on the specific affinity between a substance to be isolated and a molecule that it can specifically bind to. This is a receptor-ligand type interaction. The column material is synthesized by covalently coupling one of the binding partners to an insoluble matrix. The column material is then able to specifically adsorb the substance from the solution. Elution occurs by changing the conditions to those in which binding will not occur (alter pH, ionic strength, temperature, etc.).

A particular type of affinity chromatography useful in the purification of carbohydrate containing compounds is lectin affinity chromatography. Lectins are a class of substances that bind to a variety of polysaccharides and glycoproteins. Lectins are usually coupled to agarose by cyanogen bromide. Conconavalin A coupled to Sepharose was the first material of this sort to be used and has been widely used in the isolation of polysaccharides and glycoproteins other lectins that have been include lentil lectin, wheat germ agglutinin which has been useful in the purification of N-acetyl glucosaminyl residues and Helix pomatia lectin. Lectins themselves are purified using affinity chromatography with carbohydrate ligands. Lactose has been used to purify lectins from castor bean and peanuts; maltose has been useful in extracting lectins from lentils and jack bean; N-acetyl-D galactosamine is used for purifying lectins from soybean; N-acetyl glucosaminyl binds to lectins from wheat germ; D-galactosamine has been used in obtaining lectins from clams and L-fuctose will bind to lectins from lotus.

The matrix should be a substance that itself does not adsorb molecules to any significant extent and that has a broad range of chemical, physical and thermal stability. The ligand should be coupled in such a way as to not affect its binding properties. The ligand should also provide relatively tight binding. And it should be possible to elute the substance without destroying the sample or the ligand. One of the most common forms of affinity chromatography is immunoaffinity chromatography. The generation of antibodies that would be suitable for use in accord with the present invention is discussed below.

F. Synthetic Peptides

Because of their relatively small size, t-DARPP peptides of the invention can be synthesized in solution or on a solid support in accordance with conventional techniques. Various automatic synthesizers are commercially available and can be used in accordance with known protocols. See, for example, Stewart and Young (1984); Tam et al. (1983); Merrifield (1986); and Barany and Merrifield (1979), each incorporated herein by reference. Short peptide sequences, or libraries of overlapping peptides, usually from about 6 up to about 35 to 50 amino acids, which correspond to the selected regions described herein, can be readily synthesized and then screened in screening assays designed to identify reactive peptides. Alternatively, recombinant DNA technology may be employed wherein a nucleotide sequence which encodes a peptide of the invention is inserted into an expression vector, transformed or transfected into an appropriate host cell and cultivated under conditions suitable for expression.

G. Antigen Compositions

The present invention also provides for the use of t-DARPP proteins or peptides as antigens for the immunization of animals relating to the production of antibodies. It is envisioned that either t-DARPP, or portions thereof, will be coupled, bonded, bound, conjugated or chemically-linked to one or more agents via linkers, polylinkers or derivatized amino acids. This may be performed such that a bispecific or multivalent composition or vaccine is produced. It is further envisioned that the methods used in the preparation of these compositions will be familiar to those of skill in the art and should be suitable for administration to animals, i.e., pharmaceutically acceptable. Preferred agents are the carriers are keyhole limpet hemocyannin (KLH) or bovine serum albumin (BSA).

### III. Nucleic Acids

The present invention also provides, in another embodiment, nucleic acids encoding t-DARPP and fragments thereof. Similarly, any reference to a nucleic acid should be read as encompassing a host cell containing that nucleic acid and, in some cases, capable of expressing the product of that nucleic acid. In addition to therapeutic considerations, cells expressing nucleic acids of the present invention may prove useful in the context of screening for agents that induce, repress, inhibit, augment, interfere with, block, abrogate, stimulate or enhance the function of t-DARPP. The mRNA of DARPP-32, including the untranslated 3′ and 5′ ends, is 1983 bp, and the mRNA of t-DARPP is 1502 bp. DARPP-32 and t-DARPP share an identical sequence from exon 2 to the 3′ end. Exon 1 of t-DARPP is spliced from the intron 1 of DARPP-32.

A. Nucleic Acids Encoding t-DARPP

Nucleic acids according to the present invention may encode an entire t-DARPP gene, a domain of t-DARPP that is a competitive inhibitor of t-DARPP function, or any other fragment of the t-DARPP sequences set forth herein. The nucleic acid may be derived from genomic DNA, i.e., cloned directly from the genome of a particular organism. In preferred embodiments, however, the nucleic acid would comprise complementary DNA (cDNA). Also contemplated is a cDNA plus a natural intron or an intron derived from another gene; such engineered molecules are sometime referred to as “mini-genes.” At a minimum, these and other nucleic acids of the present invention may be used as molecular weight standards in, for example, gel electrophoresis.

The term “cDNA” is intended to refer to DNA prepared using messenger RNA (mRNA) as template. The advantage of using a cDNA, as opposed to genomic DNA or DNA polymerized from a genomic, non- or partially-processed RNA template, is that the cDNA primarily contains coding sequences of the corresponding protein. There may be times when the full or partial genomic sequence is preferred, such as where the non-coding regions are required for optimal expression or where non-coding regions such as introns are to be targeted in an antisense strategy.

As used in this application, the term “a nucleic acid encoding a t-DARPP” refers to a nucleic acid molecule that has been isolated free of total cellular nucleic acid. In certain embodiments, the invention concerns a nucleic acid sequence essentially as set forth in SEQ ID NO:1. The term “as set forth in SEQ ID NO:1” means that the nucleic acid sequence substantially corresponds to a portion of SEQ ID NO:1. The term “functionally equivalent codon” is used herein to refer to codons that encode the same amino acid, such as the six codons for arginine or serine, and also refers to codons that encode biologically equivalent amino acids, as discussed in the following pages.

Allowing for the degeneracy of the genetic code, sequences that have at least about 50%, usually at least about 60%, more usually about 70%, most usually about 80%, preferably at least about 90% and most preferably about 95% of nucleotides that are identical to the nucleotides of SEQ ID NO: 1. Sequences that are essentially the same as those set forth in SEQ ID NO: 1 also may be functionally defined as sequences that are capable of hybridizing to a nucleic acid segment containing the complement of SEQ ID NO: 1 under standard conditions.

The DNA segments of the present invention include those encoding biologically functional equivalent t-DARPP proteins and peptides, as described above. Such sequences may arise as a consequence of codon redundancy and amino acid functional equivalency that are known to occur naturally within nucleic acid sequences and the proteins thus encoded. Alternatively, functionally equivalent proteins or peptides may be created via the application of recombinant DNA technology, in which changes in the protein structure may be engineered, based on considerations of the properties of the amino acids being exchanged. Changes designed by man may be introduced through the application of site-directed mutagenesis techniques or may be introduced randomly and screened later for the desired function, as described below.

B. Oligonucleotide Probes and Primers

Naturally, the present invention also encompasses DNA segments that are complementary, or essentially complementary, to the sequence set forth in SEQ ID NO: 1. Nucleic acid sequences that are “complementary” are those that are capable of base-pairing according to the standard Watson-Crick complementary rules. As used herein, the term “complementary sequences” means nucleic acid sequences that are substantially complementary, as may be assessed by the same nucleotide comparison set forth above, or as defined as being capable of hybridizing to the nucleic acid segment of SEQ ID NO:1 under relatively stringent conditions such as those described herein. Such sequences may encode the entire t-DARPP protein or functional or non-functional fragments thereof.

Alternatively, the hybridizing segments may be shorter oligonucleotides. Sequences of 17 bases long should occur only once in the human genome and, therefore, suffice to specify a unique target sequence. Although shorter oligomers are easier to make and increase in vivo accessibility, numerous other factors are involved in determining the specificity of hybridization. Both binding affinity and sequence specificity of an oligonucleotide to its complementary target increases with increasing length. It is contemplated that exemplary oligonucleotides of 8, 9, 10, 11, 12, 13, 14, 15, 16, 17, 18, 19, 20, 25, 30, 35, 40, 45, 50, 55, 60, 65, 70, 75, 80, 85, 90, 95, 100 or more base pairs will be used, although others are contemplated. Longer polynucleotides encoding 250, 500, 1000, 1212, 1500, 2000, 2500, 3000 or longer are contemplated as well. Such oligonucleotides will find use, for example, as probes in Southern and Northern blots and as primers in amplification reactions.

Suitable hybridization conditions will be well known to those of skill in the art. In certain applications, for example, substitution of amino acids by site-directed mutagenesis, it is appreciated that lower stringency conditions are required. Under these conditions, hybridization may occur even though the sequences of probe and target strand are not perfectly complementary, but are mismatched at one or more positions. Conditions may be rendered less stringent by increasing salt concentration and decreasing temperature. For example, a medium stringency condition could be provided by about 0.1 to 0.25 M NaCl at temperatures of about 37° C. to about 55° C., while a low stringency condition could be provided by about 0.15 M to about 0.9 M salt, at temperatures ranging from about 20° C. to about 55° C. Thus, hybridization conditions can be readily manipulated, and thus will generally be a method of choice depending on the desired results.

In other embodiments, hybridization may be achieved under conditions of, for example, 50 mM Tris-HCl (pH 8.3), 75 mM KCl, 3 mM MgCl₂, 10 mM dithiothreitol, at temperatures between approximately 20° C. to about 37° C. Other hybridization conditions utilized could include approximately 10 mM Tris-HCl (pH 8.3), 50 mM KCl, 1.5 μM MgCl₂, at temperatures ranging from approximately 40° C. to about 72° C. Formamide and SDS also may be used to alter the hybridization conditions.

One method of using probes and primers of the present invention is in the search for genes related to t-DARPP or, more particularly, homologs of t-DARPP from other species. Normally, the target DNA will be a genomic or cDNA library, although screening may involve analysis of RNA molecules. By varying the stringency of hybridization, and the region of the probe, different degrees of homology may be discovered.

Another way of exploiting probes and primers of the present invention is in site-directed or site-specific mutagenesis. Site-specific mutagenesis is a technique useful in the preparation of individual peptides, or biologically functional equivalent proteins or peptides, through specific mutagenesis of the underlying DNA. The technique further provides a ready ability to prepare and test sequence variants, incorporating one or more of the foregoing considerations, by introducing one or more nucleotide sequence changes into the DNA. Site-specific mutagenesis allows the production of mutants through the use of specific oligonucleotide sequences which encode the DNA sequence of the desired mutation, as well as a sufficient number of adjacent nucleotides, to provide a primer sequence of sufficient size and sequence complexity to form a stable duplex on both sides of the deletion junction being traversed. Typically, a primer of about 17 to 25 nucleotides in length is preferred, with about 5 to 10 residues on both sides of the junction of the sequence being altered.

The technique typically employs a bacteriophage vector that exists in both a single stranded and double stranded form. Typical vectors useful in site-directed mutagenesis include vectors such as the M13 phage. These phage vectors are commercially available and their use is generally well known to those skilled in the art. Double-stranded plasmids are also routinely employed in site directed mutagenesis, which eliminates the step of transferring the gene of interest from a phage to a plasmid.

In general, site-directed mutagenesis is performed by first obtaining a single-stranded vector, or melting of two strands of a double-stranded vector which includes within its sequence a DNA sequence encoding the desired protein. An oligonucleotide primer bearing the desired mutated sequence is synthetically prepared. This primer is then annealed with the single-stranded DNA preparation, taking into account the degree of mismatch when selecting hybridization conditions, and subjected to DNA polymerizing enzymes such as E. coli polymerase I Klenow fragment, in order to complete the synthesis of the mutation-bearing strand. Thus, a heteroduplex is formed wherein one strand encodes the original non-mutated sequence and the second strand bears the desired mutation. This heteroduplex vector is then used to transform appropriate cells, such as E. coli cells, and clones are selected that include recombinant vectors bearing the mutated sequence arrangement.

The preparation of sequence variants of the selected gene using site-directed mutagenesis is provided as a means of producing potentially useful species and is not meant to be limiting, as there are other ways in which sequence variants of genes may be obtained. For example, recombinant vectors encoding the desired gene may be treated with mutagenic agents, such as hydroxylamine, to obtain sequence variants.

C. Antisense Constructs

Antisense methodology takes advantage of the fact that nucleic acids tend to pair with “complementary” sequences. By complementary, it is meant that polynucleotides are those which are capable of base-pairing according to the standard Watson-Crick complementarity rules. That is, the larger purines will base pair with the smaller pyrimidines to form combinations of guanine paired with cytosine (G:C) and adenine paired with either thymine (A:T) in the case of DNA, or adenine paired with uracil (A:U) in the case of RNA. Inclusion of less common bases such as inosine, 5-methylcytosine, 6-methyladenine, hypoxanthine and others in hybridizing sequences does not interfere with pairing.

Targeting double-stranded (ds) DNA with polynucleotides leads to triple-helix formation; targeting RNA will lead to double-helix formation. Antisense polynucleotides, when introduced into a target cell, specifically bind to their target polynucleotide and interfere with transcription, RNA processing, transport, translation and/or stability. Antisense RNA constructs, or DNA encoding such antisense RNA's, may be employed to inhibit gene transcription or translation or both within a host cell, either in vitro or in vivo, such as within a host animal, including a human subject.

Antisense constructs may be designed to bind to the promoter and other control regions, exons, introns or even exon-intron boundaries of a gene. It is contemplated that the most effective antisense constructs will include regions complementary to intron/exon splice junctions. Thus, it is proposed that a preferred embodiment includes an antisense construct with complementarity to regions within 50-200 bases of an intron-exon splice junction. It has been observed that some exon sequences can be included in the construct without seriously affecting the target selectivity thereof. The amount of exonic material included will vary depending on the particular exon and intron sequences used. One can readily test whether too much exon DNA is included simply by testing the constructs in vitro to determine whether normal cellular function is affected or whether the expression of related genes having complementary sequences is affected.

As stated above, “complementary” or “antisense” means polynucleotide sequences that are substantially complementary over their entire length and have very few base mismatches. For example, sequences of fifteen bases in length may be termed complementary when they have complementary nucleotides at thirteen or fourteen positions. Naturally, sequences which are completely complementary will be sequences which are entirely complementary throughout their entire length and have no base mismatches. Other sequences with lower degrees of homology also are contemplated. For example, an antisense construct which has limited regions of high homology, but also contains a non-homologous region (e.g., ribozyme; see below) could be designed. These molecules, though having less than 50% homology, would bind to target sequences under appropriate conditions.

It may be advantageous to combine portions of genomic DNA with cDNA or synthetic sequences to generate specific constructs. For example, where an intron is desired in the ultimate construct, a genomic clone will need to be used. The cDNA or a synthesized polynucleotide may provide more convenient restriction sites for the remaining portion of the construct and, therefore, would be used for the rest of the sequence.

D. Ribozymes

Ribozymes are RNA-protein complexes that cleave nucleic acids in a site-specific fashion. Ribozymes have specific catalytic domains that possess endonuclease activity (Kim and Cech, 1987; Gerlach et al., 1987; Forster and Symons, 1987). For example, a large number of ribozymes accelerate phosphoester transfer reactions with a high degree of specificity, often cleaving only one of several phosphoesters in an oligonucleotide substrate (Cook et al., 1981; Michel and Westhof, 1990; Reinhold-Hurek and Shub, 1992). This specificity has been attributed to the requirement that the substrate bind via specific base-pairing interactions to the internal guide sequence (“IGS”) of the ribozyme prior to chemical reaction.

Ribozyme catalysis has primarily been observed as part of sequence-specific cleavage/ligation reactions involving nucleic acids (Joyce, 1989; Cook et al., 1981). For example, U.S. Pat. No. 5,354,855 reports that certain ribozymes can act as endonucleases with a sequence specificity greater than that of known ribonucleases and approaching that of the DNA restriction enzymes. Thus, sequence-specific ribozyme-mediated inhibition of gene expression may be particularly suited to therapeutic applications (Scanlon et al., 1991; Sarver et al., 1990). Recently, it was reported that ribozymes elicited genetic changes in some cells lines to which they were applied; the altered genes included the oncogenes H-ras, c-fos and genes of HIV. Most of this work involved the modification of a target mRNA, based on a specific mutant codon that is cleaved by a specific ribozyme.

E. Interfering RNAs

In certain embodiments of the present invention, the therapeutic nucleic acid of the pharmaceutical compositions set forth herein is an RNAi. RNA interference (also referred to as “RNA-mediated interference” or RNAi) is a mechanism by which gene expression can be reduced or eliminated. Double-stranded RNA (dsRNA) has been observed to mediate the reduction, which is a multi-step process. dsRNA activates post-transcriptional gene expression surveillance mechanisms that appear to function to defend cells from virus infection and transposon activity (Fire et al., 1998; Grishok et al., 2000; Ketting et al., 1999; Lin and Avery et al., 1999; Montgomery et al., 1998; Sharp and Zamore, 2000; Tabara et al., 1999). Activation of these mechanisms targets mature, dsRNA-complementary mRNA for destruction. RNAi offers major experimental advantages for study of gene function. These advantages include a very high specificity, ease of movement across cell membranes, and prolonged down-regulation of the targeted gene (Fire et al., 1998; Grishok et al., 2000; Ketting et al., 1999; Lin and Avery et al., 1999; Montgomery et al., 1998; Sharp et al., 1999; Sharp and Zamore, 2000; Tabara et al., 1999). Moreover, dsRNA has been shown to silence genes in a wide range of systems, including plants, protozoans, fungi, C. elegans, Trypanasoma, Drosophila, and mammals (Grishok et al., 2000; Sharp et al., 1999; Sharp and Zamore, 2000; Elbashir et al., 2001). It is generally accepted that RNAi acts post-transcriptionally, targeting RNA transcripts for degradation. It appears that both nuclear and cytoplasmic RNA can be targeted (Bosher and Labouesse, 2000).

The endoribonuclease Dicer is known to produce two types of small regulatory RNAs that regulate gene expression: small interfering RNAs (siRNAs) and microRNAs (miRNAs) (Bernstein et al., 2001; Grishok et al., 2000; Hutvgner et al., 2001; Ketting et al., 2001; Knight and Bass, 2001). In animals, siRNAs direct target mRNA cleavage (Elbashir et al., 2001), whereas miRNAs block target mRNA translation (Reinhart et al., 2000; Brennecke et al., 2003; Xu et al., 2003). Recent data suggest that both siRNAs and miRNAs incorporate into similar perhaps even identical protein complexes, and that a critical determinant of mRNA destruction versus translation regulation is the degree of sequence complementary between the small RNA and its mRNA target (Hutvgner and Zamore, 2002; Mourelatos et al., 2002; Zeng et al., 2002; Doench et al., 2003; Saxena et al., 2003). Many known miRNA sequences and their position in genomes or chromosomes can be found on the world-wide-web sanger.ac.uk/Software/Rfam/mirna/help/summary.shtml.

siRNAs must be designed so that they are specific and effective in suppressing the expression of the genes of interest. Methods of selecting the target sequences, i.e., those sequences present in the gene or genes of interest to which the siRNAs will guide the degradative machinery, are directed to avoiding sequences that may interfere with the siRNA's guide function while including sequences that are specific to the gene or genes. Typically, siRNA target sequences of about 21 to 23 nucleotides in length are most effective. This length reflects the lengths of digestion products resulting from the processing of much longer RNAs as described above (Montgomery et al., 1998).

The making of siRNAs has been mainly through direct chemical synthesis; through processing of longer, double-stranded RNAs through exposure to Drosophila embryo lysates; or through an in vitro system derived from S2 cells. Use of cell lysates or in vitro processing may further involve the subsequent isolation of the short, 21-23 nucleotide siRNAs from the lysate, etc., making the process somewhat cumbersome and expensive. Chemical synthesis proceeds by making two single stranded RNA-oligomers followed by the annealing of the two single stranded oligomers into a double-stranded RNA. Methods of chemical synthesis are diverse. Non-limiting examples are provided in U.S. Pat. Nos. 5,889,136, 4,415,723, and 4,458,066, expressly incorporated herein by reference, and in Wincott et al. (1995).

Several further modifications to siRNA sequences have been suggested in order to alter their stability or improve their effectiveness. It is suggested that synthetic complementary 21-mer RNAs having di-nucleotide overhangs (i.e., 19 complementary nucleotides +3′ non-complementary dimers) may provide the greatest level of suppression. These protocols primarily use a sequence of two (2′-deoxy) thymidine nucleotides as the di-nucleotide overhangs. These dinucleotide overhangs are often written as dTdT to distinguish them from the typical nucleotides incorporated into RNA. The literature has indicated that the use of dT overhangs is primarily motivated by the need to reduce the cost of the chemically synthesized RNAs. It is also suggested that the dTdT overhangs might be more stable than UU overhangs, though the data available shows only a slight (<20%) improvement of the dTdT overhang compared to an siRNA with a UU overhang.

Chemically synthesized siRNAs are found to work optimally when they are in cell culture at concentrations of 25-100 nM, but concentrations of about 100 nM have achieved effective suppression of expression in mammalian cells. siRNAs have been most effective in mammalian cell culture at about 100 nM. In several instances, however, lower concentrations of chemically synthesized siRNA have been used (Caplen et al., 2000; Elbashir et al., 2001).

WO 99/32619 and WO 01/68836 suggest that RNA for use in siRNA may be chemically or enzymatically synthesized. Both of these texts are incorporated herein in their entirety by reference. The enzymatic synthesis contemplated in these references is by a cellular RNA polymerase or a bacteriophage RNA polymerase (e.g., T3, T7, SP6) via the use and production of an expression construct as is known in the art. For example, see U.S. Pat. No. 5,795,715. The contemplated constructs provide templates that produce RNAs that contain nucleotide sequences identical to a portion of the target gene. The length of identical sequences provided by these references is at least 25 bases, and may be as many as 400 or more bases in length. An important aspect of this reference is that the authors contemplate digesting longer dsRNAs to 21-25mer lengths with the endogenous nuclease complex that converts long dsRNAs to siRNAs in vivo. They do not describe or present data for synthesizing and using in vitro transcribed 21-25mer dsRNAs. No distinction is made between the expected properties of chemical or enzymatically synthesized dsRNA in its use in RNA interference.

Similarly, WO 00/44914, incorporated herein by reference, suggests that single strands of RNA can be produced enzymatically or by partial/total organic synthesis. Preferably, single-stranded RNA is enzymatically synthesized from the PCR products of a DNA template, preferably a cloned cDNA template and the RNA product is a complete transcript of the cDNA, which may comprise hundreds of nucleotides. WO 01/36646, incorporated herein by reference, places no limitation upon the manner in which the siRNA is synthesized, providing that the RNA may be synthesized in vitro or in vivo, using manual and/or automated procedures. This reference also provides that in vitro synthesis may be chemical or enzymatic, for example using cloned RNA polymerase (e.g., T3, T7, SP6) for transcription of the endogenous DNA (or cDNA) template, or a mixture of both. Again, no distinction in the desirable properties for use in RNA interference is made between chemically or enzymatically synthesized siRNA.

U.S. Pat. No. 5,795,715 reports the simultaneous transcription of two complementary DNA sequence strands in a single reaction mixture, wherein the two transcripts are immediately hybridized. The templates used are preferably of between 40 and 100 base pairs, and which is equipped at each end with a promoter sequence. The templates are preferably attached to a solid surface. After transcription with RNA polymerase, the resulting dsRNA fragments may be used for detecting and/or assaying nucleic acid target sequences.

U.S. Patent Publication 2005/0203047 reports of a method of modulating gene expression through RNA interference by incorporating a siRNA or miRNA sequence into a transfer RNA (tRNA) encoding sequence. The tRNA containing the siRNA or miRNA sequence may be incorporated into a nucleic acid expression construct so that this sequence is spliced from the expressed tRNA. The siRNA or miRNA sequence may be positioned within an intron associated with an unprocessed tRNA transcript, or may be positioned at either end of the tRNA transcript.

Interfering RNAs may comprise one or more modified nucleotides, such as 2′-O-methyl-sugar modifications. In some embodiments, antagomirs comprise only modified nucleotides. Interfering RNAs may also comprise one or more phosphorothioate linkages resulting in a partial or full phosphorothioate backbone. To facilitate in vivo delivery and stability, the interfering RNA may be linked to a cholesterol moiety at its 3′ end. Interfering RNAs may be about 15 to about 50 nucleotides in length, more preferably about 18 to about 30 nucleotides in length, and most preferably about 20 to about 25 nucleotides in length. “Partially complementary” refers to a sequence that is at least about 75%, 80%, 85%, 90%, 95%, 96%, 97%, 98%, or 99% complementary to a target polynucleotide sequence.

F. Vectors for Cloning, Gene Transfer and Expression

Within certain embodiments, expression vectors are employed to express a t-DARPP polypeptide or peptide product, an antisene, a ribozyme, an interfering RNA, or a single-chain antibody that binds immulogically to t-DARPP. In other embodiments, the expression vectors are used in gene therapy. Expression requires that appropriate signals be provided in the vectors, and which include various regulatory elements, such as enhancers/promoters from both viral and mammalian sources that drive expression of the genes of interest in host cells. Elements designed to optimize messenger RNA stability and translatability in host cells also are defined. The conditions for the use of a number of dominant drug selection markers for establishing permanent, stable cell clones expressing the products are also provided, as is an element that links expression of the drug selection markers to expression of the polypeptide.

Throughout this application, the term “expression construct” is meant to include any type of genetic construct containing a nucleic acid coding for a gene product in which part or all of the nucleic acid encoding sequence is capable of being transcribed. The transcript may be translated into a protein, but it need not be. In certain embodiments, expression includes both transcription of a gene and translation of mRNA into a gene product. In other embodiments, expression only includes transcription of the nucleic acid encoding a gene of interest.

The term “vector” is used to refer to a carrier nucleic acid molecule into which a nucleic acid sequence can be inserted for introduction into a cell where it can be replicated. A nucleic acid sequence can be “exogenous,” which means that it is foreign to the cell into which the vector is being introduced or that the sequence is homologous to a sequence in the cell but in a position within the host cell nucleic acid in which the sequence is ordinarily not found. Vectors include plasmids, cosmids, viruses (bacteriophage, animal viruses, and plant viruses), and artificial chromosomes (e.g., YACs). One of skill in the art would be well equipped to construct a vector through standard recombinant techniques, which are described in Sambrook et al. (1989) and Ausubel et al. (1994), both incorporated herein by reference.

The term “expression vector” refers to a vector containing a nucleic acid sequence coding for at least part of a gene product capable of being transcribed. In some cases, RNA molecules are then translated into a protein, polypeptide, or peptide. In other cases, these sequences are not translated, for example, in the production of antisense molecules or ribozymes. Expression vectors can contain a variety of “control sequences,” which refer to nucleic acid sequences necessary for the transcription and possibly translation of an operably linked coding sequence in a particular host organism. In addition to control sequences that govern transcription and translation, vectors and expression vectors may contain nucleic acid sequences that serve other functions as well and are described infra.

(i) Regulatory Elements

A “promoter” is a control sequence that is a region of a nucleic acid sequence at which initiation and rate of transcription are controlled. It may contain genetic elements at which regulatory proteins and molecules may bind such as RNA polymerase and other transcription factors. The phrases “operatively positioned,” “operatively linked,” “under control,” and “under transcriptional control” mean that a promoter is in a correct functional location and/or orientation in relation to a nucleic acid sequence to control transcriptional initiation and/or expression of that sequence. A promoter may or may not be used in conjunction with an “enhancer,” which refers to a cis-acting regulatory sequence involved in the transcriptional activation of a nucleic acid sequence.

A promoter may be one naturally-associated with a gene or sequence, as may be obtained by isolating the 5′ non-coding sequences located upstream of the coding segment and/or exon. Such a promoter can be referred to as “endogenous.” Similarly, an enhancer may be one naturally associated with a nucleic acid sequence, located either downstream or upstream of that sequence. Alternatively, certain advantages will be gained by positioning the coding nucleic acid segment under the control of a recombinant or heterologous promoter, which refers to a promoter that is not normally associated with a nucleic acid sequence in its natural environment. A recombinant or heterologous enhancer refers also to an enhancer not normally associated with a nucleic acid sequence in its natural environment. Such promoters or enhancers may include promoters or enhancers of other genes, and promoters or enhancers isolated from any other prokaryotic, viral, or eukaryotic cell, and promoters or enhancers not “naturally-occurring,” i.e., containing different elements of different transcriptional regulatory regions, and/or mutations that alter expression. In addition to producing nucleic acid sequences of promoters and enhancers synthetically, sequences may be produced using recombinant cloning and/or nucleic acid amplification technology, including PCR™, in connection with the compositions disclosed herein (see U.S. Pat. No. 4,683,202, U.S. Pat. No. 5,928,906, each incorporated herein by reference). Furthermore, it is contemplated the control sequences that direct transcription and/or expression of sequences within non-nuclear organelles such as mitochondria, chloroplasts, and the like, can be employed as well.

Naturally, it will be important to employ a promoter and/or enhancer that effectively directs the expression of the DNA segment in the cell type, organelle, and organism chosen for expression. One example is the native t-Darpp promoter. Those of skill in the art of molecular biology generally know the use of promoters, enhancers, and cell type combinations for protein expression, for example, see Sambrook et al. (1989), incorporated herein by reference. The promoters employed may be constitutive, tissue-specific, inducible, and/or useful under the appropriate conditions to direct high level expression of the introduced DNA segment, such as is advantageous in the large-scale production of recombinant proteins and/or peptides. The promoter may be heterologous or endogenous.

Table 2 lists several elements/promoters that may be employed, in the context of the present invention, to regulate the expression of a gene. This list is not intended to be exhaustive of all the possible elements involved in the promotion of expression but, merely, to be exemplary thereof. Table 3 provides examples of inducible elements, which are regions of a nucleic acid sequence that can be activated in response to a specific stimulus.

TABLE 2
Promoter and/or Enhancer

| Promoter/Enhancer | References |
|---|---|
| Immunoglobulin Heavy Chain | Banerji et al., 1983; Gilles et al., 1983; Grosschedl et al., |
| | 1985; Atchinson et al., 1986, 1987; Imler et al., 1987; |
| | Weinberger et al., 1984; Kiledjian et al., 1988; Porton et |
| | al; 1990 |
| Immunoglobulin Light Chain | Queen et al., 1983; Picard et al., 1984 |
| T-Cell Receptor | Luria et al., 1987; Winoto et al., 1989; Redondo et al; |
| | 1990 |
| HLA DQ a and/or DQ β | Sullivan et al., 1987 |
| β-Interferon | Goodbourn et al., 1986; Fujita et al., 1987; Goodbourn et |
| | al., 1988 |
| Interleukin-2 | Greene et al., 1989 |
| Interleukin-2 Receptor | Greene et al., 1989; Lin et al., 1990 |
| MHC Class II 5 | Koch et al., 1989 |
| MHC Class II HLA-DRa | Sherman et al., 1989 |
| β-Actin | Kawamoto et al., 1988; Ng et al.; 1989 |
| Muscle Creatine Kinase (MCK) | Jaynes et al., 1988; Horlick et al., 1989; Johnson et al., |
| | 1989 |
| Prealbumin (Transthyretin) | Costa et al., 1988 |
| Elastase I | Ornitz et al., 1987 |
| Metallothionein (MTII) | Karin et al., 1987; Culotta et al., 1989 |
| Collagenase | Pinkert et al., 1987; Angel et al., 1987 |
| Albumin | Pinkert et al., 1987; Tronche et al., 1989, 1990 |
| α-Fetoprotein | Godbout et al., 1988; Campere et al., 1989 |
| t-Globin | Bodine et al., 1987; Perez-Stable et al., 1990 |
| β-Globin | Trudel et al., 1987 |
| c-fos | Cohen et al., 1987 |
| c-HA-ras | Triesman, 1986; Deschamps et al., 1985 |
| Insulin | Edlund et al., 1985 |
| Neural Cell Adhesion Molecule (NCAM) | Hirsh et al., 1990 |
| α₁-Antitrypain | Latimer et al., 1990 |
| H2B (TH2B) Histone | Hwang et al., 1990 |
| Mouse and/or Type I Collagen | Ripe et al., 1989 |
| Glucose-Regulated Proteins | Chang et al., 1989 |
| (GRP94 and GRP78) | |
| Rat Growth Hormone | Larsen et al., 1986 |
| Human Serum Amyloid A (SAA) | Edbrook et al., 1989 |
| Troponin I (TN I) | Yutzey et al., 1989 |
| Platelet-Derived Growth Factor | Pech et al., 1989 |
| (PDGF) | |
| Duchenne Muscular Dystrophy | Klamut et al., 1990 |
| SV40 | Banerji et al., 1981; Moreau et al., 1981; Sleigh et al., |
| | 1985; Firak et al., 1986; Herr et al., 1986; Imbra et al., |
| | 1986; Kadesch et al., 1986; Wang et al., 1986; Ondek et |
| | al., 1987; Kuhl et al., 1987; Schaffner et al., 1988 |
| Polyoma | Swartzendruber et al., 1975; Vasseur et al., 1980; Katinka |
| | et al., 1980, 1981; Tyndell et al., 1981; Dandolo et al., |
| | 1983; de Villiers et al., 1984; Hen et al., 1986; Satake et |
| | al., 1988; Campbell and/or Villarreal, 1988 |
| Retroviruses | Kriegler et al., 1982, 1983; Levinson et al., 1982; Kriegler |
| | et al., 1983, 1984a, b, 1988; Bosze et al., 1986; Miksicek |
| | et al., 1986; Celander et al., 1987; Thiesen et al., 1988; |
| | Celander et al., 1988; Choi et al., 1988; Reisman et al., |
| | 1989 |
| Papilloma Virus | Campo et al., 1983; Lusky et al., 1983; Spandidos and/or |
| | Wilkie, 1983; Spalholz et al., 1985; Lusky et al., 1986; |
| | Cripe et al., 1987; Gloss et al., 1987; Hirochika et al., |
| | 1987; Stephens et al., 1987; Glue et al., 1988 |
| Hepatitis B Virus | Bulla et al., 1986; Jameel et al., 1986; Shaul et al., 1987; |
| | Spandau et al., 1988; Vannice et al., 1988 |
| Human Immunodeficiency Virus | Muesing et al., 1987; Hauber et al., 1988; Jakobovits et |
| | al., 1988; Feng et al., 1988; Takebe et al., 1988; Rosen et |
| | al., 1988; Berkhout et al., 1989; Laspia et al., 1989; Sharp |
| | et al., 1989; Braddock et al., 1989 |
| Cytomegalovirus (CMV) | Weber et al., 1984; Boshart et al., 1985; Foecking et al., |
| | 1986 |
| Gibbon Ape Leukemia Virus | Holbrook et al., 1987; Quinn et al., 1989 |

TABLE 3
Inducible Elements

| Element | Inducer | References |
|---|---|---|
| MT II | Phorbol Ester (TFA) | Palmiter et al., 1982; Haslinger et |
| | Heavy metals | al., 1985; Searle et al., 1985; |
| | | Stuart et al., 1985; Imagawa et |
| | | al., 1987, Karin et al., 1987; |
| | | Angel et al., 1987b; McNeall et |
| | | al., 1989 |
| MMTV (mouse mammary | Glucocorticoids | Huang et al., 1981; Lee et al., |
| tumor virus) | | 1981; Majors et al., 1983; |
| | | Chandler et al., 1983; Lee et al., |
| | | 1984; Ponta et al., 1985; Sakai et |
| | | al., 1988 |
| β-Interferon | poly(rI)x | Tavernier et al., 1983 |
| | poly(rc) | |
| Adenovirus 5 E2 | ElA | Imperiale et al., 1984 |
| Collagenase | Phorbol Ester (TPA) | Angel et al., 1987a |
| Stromelysin | Phorbol Ester (TPA) | Angel et al., 1987b |
| SV40 | Phorbol Ester (TPA) | Angel et al., 1987b |
| Murine MX Gene | Interferon, Newcastle | Hug et al., 1988 |
| | Disease Virus | |
| GRP78 Gene | A23187 | Resendez et at., 1988 |
| α-2-Macroglobulin | IL-6 | Kunz et al., 1989 |
| Vimentin | Serum | Rittling et al., 1989 |
| MHC Class I Gene H-2κb | Interferon | Blanar et al., 1989 |
| HSP70 | ElA, SV40 Large T | Taylor et al., 1989, 1990a, 1990b |
| | Antigen | |
| Proliferin | Phorbol Ester-TPA | Mordacq et al., 1989 |
| Tumor Necrosis Factor | PMA | Hensel et al., 1989 |
| Thyroid Stimulating | Thyroid Hormone | Charterjee et al., 1989 |
| Hormone α Gene | | |

The identity of tissue-specific promoters or elements, as well as assays to characterize their activity, is well known to those of skill in the art. Examples of such regions include the human LIMK2 gene (Nomoto et al. 1999), the somatostatin receptor 2 gene (Kraus et al., 1998), murine epididymal retinoic acid-binding gene (Lareyre et al., 1999), human CD4 (Zhao-Emonet et al., 1998), mouse alpha2 (XI) collagen (Tsumaki, et al., 1998), D1A dopamine receptor gene (Lee, et al., 1997), insulin-like growth factor II (Wu et al., 1997), human platelet endothelial cell adhesion molecule-1 (Almendro et al., 1996). Tumor specific promoters also will find use in the present invention. Some such promoters are set forth in Tables 4 and 5.

TABLE 4
Candidate Tissue-Specific Promoters for Cancer Gene Therapy

| | Cancers in which promoter | Normal cells in which |
|---|---|---|
| Tissue-specific promoter | is active | promoter is active |
| Carcinoembryonic antigen | Most colorectal carcinomas; | Colonic mucosa; gastric |
| (CEA)* | 50% of lung carcinomas; | mucosa; lung epithelia; |
| | 40-50% of gastric carcinomas; | eccrine sweat glands; |
| | most pancreatic carcinomas; | cells in testes |
| | many breast carcinomas | |
| Prostate-specific antigen | Most prostate carcinomas | Prostate epithelium |
| (PSA) | | |
| Vasoactive intestinal peptide | Majority of non-small cell | Neurons; lymphocytes; mast |
| (VIP) | lung cancers | cells; eosinophils |
| Surfactant protein A (SP-A) | Many lung adenocarcinomas | Type II pneumocytes; Clara |
| | cells | |
| Human achaete-scute | Most small cell lung cancers | Neuroendocrine cells in lung |
| homolog (hASH) | | |
| Mucin-1 (MUC1)** | Most adenocarcinomas | Glandular epithelial cells in |
| | (originating from any tissue) | breast and in respiratory, |
| | | gastrointestinal., and |
| | | genitourinary tracts |
| Alpha-fetoprotein | Most hepatocellular | Hepatocytes (under certain |
| | carcinomas; possibly many | conditions); testis |
| | testicular cancers | |
| Albumin | Most hepatocellular | Hepatocytes |
| | carcinomas | |
| Tyrosinase | Most melanomas | Melanocytes; astrocytes; |
| | | Schwann cells; some neurons |
| Tyrosine-binding protein | Most melanomas | Melanocytes; astrocytes, |
| (TRP) | | Schwann cells; some neurons |
| Keratin 14 | Presumably many squamous | Keratinocytes |
| | cell carcinomas (e.g., Head | |
| | and neck cancers) | |
| EBV LD-2 | Many squamous cell | Keratinocytes of upper |
| | carcinomas of head and neck | digestive Keratinocytes of |
| | | upper digestive tract |
| Glial fibrillary acidic protein | Many astrocytomas | Astrocytes |
| (GFAP) | | |
| Myelin basic protein (MBP) | Many gliomas | Oligodendrocytes |
| Testis-specific angiotensin- | Possibly many testicular | Spermatazoa |
| converting enzyme (Testis- | cancers | |
| specific ACE) | | |
| Osteocalcin | Possibly many osteosarcomas | Osteoblasts |

TABLE 5
Candidate Promoters for Tissue-Specific Targeting of Tumors

| | Cancers in which Promoter | Normal cells in which |
|---|---|---|
| Promoter | is active | Promoter is active |
| E2F-regulated promoter | Almost all cancers | Proliferating cells |
| HLA-G | Many colorectal carcinomas; | Lymphocytes; monocytes; |
| | many melanomas; possibly | spermatocytes; trophoblast |
| | many other cancers | |
| FasL | Most melanomas; many | Activated leukocytes: |
| | pancreatic carcinomas; most | neurons; endothelial cells; |
| | astrocytomas possibly many | keratinocytes; cells in |
| | other cancers | immunoprivileged tissues; |
| | | some cells in lungs, ovaries, |
| | | liver, and prostate |
| Myc-regulated promoter | Most lung carcinomas (both | Proliferating cells (only some |
| | small cell and non-small cell); | cell-types): mammary |
| | most colorectal carcinomas | epithelial cells (including non- |
| | | proliferating) |
| MAGE-1 | Many melanomas; some non- | Testis |
| | small cell lung carcinomas; | |
| | some breast carcinomas | |
| VEGF | 70% of all cancers | Cells at sites of |
| | (constitutive overexpression in | neovascularization (but unlike |
| | many cancers) | in tumors, expression is |
| | | transient, less strong, and |
| | | never constitutive) |
| bFGF | Presumably many different | Cells at sites of ischemia (but |
| | cancers, since bFGF | unlike tumors, expression is |
| | expression is induced by | transient, less strong, and |
| | ischemic conditions | never constitutive) |
| COX-2 | Most colorectal carcinomas; | Cells at sites of inflammation |
| | many lung carcinomas; | |
| | possibly many other cancers | |
| IL-10 | Most colorectal carcinomas; | Leukocytes |
| | many lung carcinomas; many | |
| | squamous cell carcinomas of | |
| | head and neck; possibly many | |
| | other cancers | |
| GRP78/BiP | Presumably many different | Cells at sites of ishemia |
| | cancers, since GRP7S | |
| | expression is induced by | |
| | tumor-specific conditions | |
| CarG elements from Egr-1 | Induced by ionization | Cells exposed to ionizing |
| | radiation, so conceivably most | radiation; leukocytes |
| | tumors upon irradiation | |

A specific initiation signal also may be required for efficient translation of coding sequences. These signals include the ATG initiation codon or adjacent sequences. Exogenous translational control signals, including the ATG initiation codon, may need to be provided. One of ordinary skill in the art would readily be capable of determining this and providing the necessary signals. It is well known that the initiation codon must be “in-frame” with the reading frame of the desired coding sequence to ensure translation of the entire insert. The exogenous translational control signals and initiation codons can be either natural or synthetic. The efficiency of expression may be enhanced by the inclusion of appropriate transcription enhancer elements.

(ii) IRES

In certain embodiments of the invention, the use of internal ribosome entry sites (IRES) elements are used to create multigene, or polycistronic, messages. IRES elements are able to bypass the ribosome scanning model of 5′-methylated Cap dependent translation and begin translation at internal sites (Pelletier and Sonenberg, 1988). IRES elements from two members of the picornavirus family (polio and encephalomyocarditis) have been described (Pelletier and Sonenberg, 1988), as well an IRES from a mammalian message (Macejak and Sarnow, 1991). IRES elements can be linked to heterologous open reading frames. Multiple open reading frames can be transcribed together, each separated by an IRES, creating polycistronic messages. By virtue of the IRES element, each open reading frame is accessible to ribosomes for efficient translation. Multiple genes can be efficiently expressed using a single promoter/enhancer to transcribe a single message (see U.S. Pat. Nos. 5,925,565 and 5,935,819, herein incorporated by reference).

(iii) Multi-Purpose Cloning Sites

Vectors can include a multiple cloning site (MCS), which is a nucleic acid region that contains multiple restriction enzyme sites, any of which can be used in conjunction with standard recombinant technology to digest the vector. See Carbonelli et al., 1999, Levenson et al., 1998, and Cocea, 1997, incorporated herein by reference. “Restriction enzyme digestion” refers to catalytic cleavage of a nucleic acid molecule with an enzyme that functions only at specific locations in a nucleic acid molecule. Many of these restriction enzymes are commercially available. Use of such enzymes is widely understood by those of skill in the art. Frequently, a vector is linearized or fragmented using a restriction enzyme that cuts within the MCS to enable exogenous sequences to be ligated to the vector. “Ligation” refers to the process of forming phosphodiester bonds between two nucleic acid fragments, which may or may not be contiguous with each other. Techniques involving restriction enzymes and ligation reactions are well known to those of skill in the art of recombinant technology.

(iv) Splicing Sites

Most transcribed eukaryotic RNA molecules will undergo RNA splicing to remove introns from the primary transcripts. Vectors containing genomic eukaryotic sequences may require donor and/or acceptor splicing sites to ensure proper processing of the transcript for protein expression (see Chandler et al., 1997, herein incorporated by reference).

(v) Termination Signals

The vectors or constructs of the present invention will generally comprise at least one termination signal. A “termination signal” or “terminator” is comprised of the DNA sequences involved in specific termination of an RNA transcript by an RNA polymerase. Thus, in certain embodiments a termination signal that ends the production of an RNA transcript is contemplated. A terminator may be necessary in vivo to achieve desirable message levels.

In eukaryotic systems, the terminator region may also comprise specific DNA sequences that permit site-specific cleavage of the new transcript so as to expose a polyadenylation site. This signals a specialized endogenous polymerase to add a stretch of about 200 A residues (polyA) to the 3′ end of the transcript. RNA molecules modified with this polyA tail appear to more stable and are translated more efficiently. Thus, in other embodiments involving eukaryotes, it is preferred that that terminator comprises a signal for the cleavage of the RNA, and it is more preferred that the terminator signal promotes polyadenylation of the message. The terminator and/or polyadenylation site elements can serve to enhance message levels and/or to minimize read through from the cassette into other sequences.

Terminators contemplated for use in the invention include any known terminator of transcription described herein or known to one of ordinary skill in the art, including but not limited to, for example, the termination sequences of genes, such as for example the bovine growth hormone terminator or viral termination sequences, such as for example the SV40 terminator. In certain embodiments, the termination signal may be a lack of transcribable or translatable sequence, such as due to a sequence truncation.

(vi) Polyadenylation Signals

In expression, particularly eukaryotic expression, one will typically include a polyadenylation signal to effect proper polyadenylation of the transcript. The nature of the polyadenylation signal is not believed to be crucial to the successful practice of the invention, and/or any such sequence may be employed. Preferred embodiments include the SV40 polyadenylation signal and/or the bovine growth hormone polyadenylation signal, convenient and/or known to function well in various target cells. Polyadenylation may increase the stability of the transcript or may facilitate cytoplasmic transport.

(vii) Origins of Replication

In order to propagate a vector in a host cell, it may contain one or more origins of replication sites (often termed “ori”), which is a specific nucleic acid sequence at which replication is initiated. Alternatively an autonomously replicating sequence (ARS) can be employed if the host cell is yeast.

(viii) Selectable and Screenable Markers

In certain embodiments of the invention, cells containing a nucleic acid construct of the present invention may be identified in vitro or in vivo by including a marker in the expression vector. Such markers would confer an identifiable change to the cell permitting easy identification of cells containing the expression vector. Generally, a selectable marker is one that confers a property that allows for selection. A positive selectable marker is one in which the presence of the marker allows for its selection, while a negative selectable marker is one in which its presence prevents its selection. An example of a positive selectable marker is a drug resistance marker.

Usually the inclusion of a drug selection marker aids in the cloning and identification of transformants, for example, genes that confer resistance to neomycin, puromycin, hygromycin, DHFR, GPT, zeocin and histidinol are useful selectable markers. In addition to markers conferring a phenotype that allows for the discrimination of transformants based on the implementation of conditions, other types of markers including screenable markers such as GFP, whose basis is calorimetric analysis, are also contemplated. Alternatively, screenable enzymes such as herpes simplex virus thymidine kinase (tk) or chloramphenicol acetyltransferase (CAT) may be utilized. One of skill in the art would also know how to employ immunologic markers, possibly in conjunction with FACS analysis. The marker used is not believed to be important, so long as it is capable of being expressed simultaneously with the nucleic acid encoding a gene product. Further examples of selectable and screenable markers are well known to one of skill in the art.

(ix) Viral Vectors

The capacity of certain viral vectors to efficiently infect or enter cells, to integrate into a host cell genome and stably express viral genes, have led to the development and application of a number of different viral vector systems (Robbins et al., 1998). Viral systems are currently being developed for use as vectors for ex vivo and in vivo gene transfer. For example, adenovirus, herpes-simplex virus, retrovirus and adeno-associated virus vectors are being evaluated currently for treatment of diseases such as cancer, cystic fibrosis, Gaucher disease, renal disease and arthritis (Robbins and Ghivizzani, 1998; Imai et al., 1998; U.S. Pat. No. 5,670,488). The various viral vectors described below, present specific advantages and disadvantages, depending on the particular gene-therapeutic application.

Adenoviral Vectors. In particular embodiments, an adenoviral expression vector is contemplated for the delivery of expression constructs. “Adenovirus expression vector” is meant to include those constructs containing adenovirus sequences sufficient to (a) support packaging of the construct and (b) to ultimately express a tissue or cell-specific construct that has been cloned therein.

Adenoviruses comprise linear, double-stranded DNA, with a genome ranging from 30 to 35 kb in size (Reddy et al., 1998; Morrison et al., 1997; Chillon et al., 1999). An adenovirus expression vector according to the present invention comprises a genetically engineered form of the adenovirus. Advantages of adenoviral gene transfer include the ability to infect a wide variety of cell types, including non-dividing cells, a mid-sized genome, ease of manipulation, high infectivity and the ability to be grown to high titers (Wilson, 1996). Further, adenoviral infection of host cells does not result in chromosomal integration because adenoviral DNA can replicate in an episomal manner, without potential genotoxicity associated with other viral vectors. Adenoviruses also are structurally stable (Marienfeld et al., 1999) and no genome rearrangement has been detected after extensive amplification (Parks et al., 1997; Bett et al., 1993).

Salient features of the adenovirus genome are an early region (E1, E2, E3 and E4 genes), an intermediate region (pIX gene, Iva2 gene), a late region (L1, L2, L3, L4 and L5 genes), a major late promoter (MLP), inverted-terminal-repeats (ITRs) and a ψ sequence (Zheng, et al., 1999; Robbins et al., 1998; Graham and Prevec, 1995). The early genes E1, E2, E3 and E4 are expressed from the virus after infection and encode polypeptides that regulate viral gene expression, cellular gene expression, viral replication, and inhibition of cellular apoptosis. Further on during viral infection, the MLP is activated, resulting in the expression of the late (L) genes, encoding polypeptides required for adenovirus encapsidation. The intermediate region encodes components of the adenoviral capsid. Adenoviral inverted terminal repeats (ITRs; 100-200 bp in length), are cis elements, and function as origins of replication and are necessary for viral DNA replication. The ψ sequence is required for the packaging of the adenoviral genome.

A common approach for generating adenoviruses for use as a gene transfer vectors is the deletion of the E1 gene (E1⁻), which is involved in the induction of the E2, E3 and E4 promoters (Graham and Prevec, 1995). Subsequently, a therapeutic gene or genes can be inserted recombinantly in place of the E1 gene, wherein expression of the therapeutic gene(s) is driven by the E1 promoter or a heterologous promoter. The E1⁻, replication-deficient virus is then proliferated in a “helper” cell line that provides the E1 polypeptides in trans (e.g., the human embryonic kidney cell line 293). Thus, in the present invention it may be convenient to introduce the transforming construct at the position from which the E1-coding sequences have been removed. However, the position of insertion of the construct within the adenovirus sequences is not critical to the invention. Alternatively, the E3 region, portions of the E4 region or both may be deleted, wherein a heterologous nucleic acid sequence under the control of a promoter operable in eukaryotic cells is inserted into the adenovirus genome for use in gene transfer (U.S. Pat. No. 5,670,488; U.S. Pat. No. 5,932,210, each specifically incorporated herein by reference).

Although adenovirus based vectors offer several unique advantages over other vector systems, they often are limited by vector immunogenicity, size constraints for insertion of recombinant genes and low levels of replication. The preparation of a recombinant adenovirus vector deleted of all open reading frames, comprising a full length dystrophin gene and the terminal repeats required for replication (Haecker et al., 1997) offers some potentially promising advantages to the above mentioned adenoviral shortcomings. The vector was grown to high titer with a helper virus in 293 cells and was capable of efficiently transducing dystrophin in mdx mice, in myotubes in vitro and muscle fibers in vivo. Helper-dependent viral vectors are discussed below.

A major concern in using adenoviral vectors is the generation of a replication-competent virus during vector production in a packaging cell line or during gene therapy treatment of an individual. The generation of a replication-competent virus could pose serious threat of an unintended viral infection and pathological consequences for the patient. Armentano et al. (1990), describe the preparation of a replication-defective adenovirus vector, claimed to eliminate the potential for the inadvertent generation of a replication-competent adenovirus (U.S. Pat. No. 5,824,544, specifically incorporated herein by reference). The replication-defective adenovirus method comprises a deleted E1 region and a relocated protein IX gene, wherein the vector expresses a heterologous, mammalian gene.

Other than the requirement that the adenovirus vector be replication defective, or at least conditionally defective, the nature of the adenovirus vector is not believed to be crucial to the successful practice of the invention. The adenovirus may be of any of the 42 different known serotypes and/or subgroups A-F. Adenovirus type 5 of subgroup C is the preferred starting material in order to obtain the conditional replication-defective adenovirus vector for use in the present invention. This is because adenovirus type 5 is a human adenovirus about which a great deal of biochemical and genetic information is known, and it has historically been used for most constructions employing adenovirus as a vector.

As stated above, the typical vector according to the present invention is replication defective and will not have an adenovirus E1 region. Adenovirus growth and manipulation is known to those of skill in the art, and exhibits broad host range in vitro and in vivo (U.S. Pat. No. 5,670,488; U.S. Pat. No. 5,932,210; U.S. Pat. No. 5,824,544). This group of viruses can be obtained in high titers, e.g., 10⁹ to 10¹¹ plaque-forming units per ml, and they are highly infective. The life cycle of adenovirus does not require integration into the host cell genome. The foreign genes delivered by adenovirus vectors are episomal and, therefore, have low genotoxicity to host cells. Many experiments, innovations, preclinical studies and clinical trials are currently under investigation for the use of adenoviruses as gene delivery vectors. For example, adenoviral gene delivery-based gene therapies are being developed for liver diseases (Han et al., 1999), psychiatric diseases (Lesch, 1999), neurological diseases (Smith, 1998; Hermens and Verhaagen, 1998), coronary diseases (Feldman et al., 1996), muscular diseases (Petrof, 1998), gastrointestinal diseases (Wu, 1998) and various cancers such as colorectal (Fujiwara and Tanaka, 1998; Dorai et al., 1999), pancreatic, bladder (Irie et al., 1999), head and neck (Blackwell et al., 1999), breast (Stewart et al., 1999), lung (Batra et al., 1999) and ovarian (Vanderkwaak et al., 1999).

Retroviral Vectors. In certain embodiments of the invention, the uses of retroviruses for gene delivery are contemplated. Retroviruses are RNA viruses comprising an RNA genome. When a host cell is infected by a retrovirus, the genomic RNA is reverse transcribed into a DNA intermediate which is integrated into the chromosomal DNA of infected cells. This integrated DNA intermediate is referred to as a provirus. A particular advantage of retroviruses is that they can stably infect dividing cells with a gene of interest (e.g., a therapeutic gene) by integrating into the host DNA, without expressing immunogenic viral proteins. Theoretically, the integrated retroviral vector will be maintained for the life of the infected host cell, expressing the gene of interest.

The retroviral genome and the proviral DNA have three genes: gag, pol, and env, which are flanked by two long terminal repeat (LTR) sequences. The gag gene encodes the internal structural (matrix, capsid, and nucleocapsid) proteins; the pol gene encodes the RNA-directed DNA polymerase (reverse transcriptase) and the env gene encodes viral envelope glycoproteins. The 5′ and 3′ LTRs serve to promote transcription and polyadenylation of the virion RNAs. The LTR contains all other cis-acting sequences necessary for viral replication.

A recombinant retrovirus of the present invention may be genetically modified in such a way that some of the structural, infectious genes of the native virus have been removed and replaced instead with a nucleic acid sequence to be delivered to a target cell (U.S. Pat. No. 5,858,744; U.S. Pat. No. 5,739,018, each incorporated herein by reference). After infection of a cell by the virus, the virus injects its nucleic acid into the cell and the retrovirus genetic material can integrate into the host cell genome. The transferred retrovirus genetic material is then transcribed and translated into proteins within the host cell. As with other viral vector systems, the generation of a replication-competent retrovirus during vector production or during therapy is a major concern. Retroviral vectors suitable for use in the present invention are generally defective retroviral vectors that are capable of infecting the target cell, reverse transcribing their RNA genomes, and integrating the reverse transcribed DNA into the target cell genome, but are incapable of replicating within the target cell to produce infectious retroviral particles (e.g., the retroviral genome transferred into the target cell is defective in gag, the gene encoding virion structural proteins, and/or in pol, the gene encoding reverse transcriptase). Thus, transcription of the provirus and assembly into infectious virus occurs in the presence of an appropriate helper virus or in a cell line containing appropriate sequences enabling encapsidation without coincident production of a contaminating helper virus.

The growth and maintenance of retroviruses is known in the art (U.S. Pat. No. 5,955,331; U.S. Pat. No. 5,888,502, each specifically incorporated herein by reference). Nolan et al. describe the production of stable high titre, helper-free retrovirus comprising a heterologous gene (U.S. Pat. No. 5,830,725, specifically incorporated herein by reference). Methods for constructing packaging cell lines useful for the generation of helper-free recombinant retroviruses with amphoteric or ecotrophic host ranges, as well as methods of using the recombinant retroviruses to introduce a gene of interest into eukaryotic cells in vivo and in vitro are contemplated in the present invention (U.S. Pat. No. 5,955,331).

Currently, the majority of all clinical trials for vector-mediated gene delivery use murine leukemia virus (MLV)-based retroviral vector gene delivery (Robbins et al., 1998; Miller et al., 1993). Disadvantages of retroviral gene delivery include a requirement for ongoing cell division for stable infection and a coding capacity that prevents the delivery of large genes. However, recent development of vectors such as lentivirus (e.g., HIV), simian immunodeficiency virus (SIV) and equine infectious-anemia virus (EIAV), which can infect certain non-dividing cells, potentially allow the in vivo use of retroviral vectors for gene therapy applications (Amado and Chen, 1999; Klimatcheva et al., 1999; White et al., 1999; Case et al., 1999). For example, HIV-based vectors have been used to infect non-dividing cells such as neurons (Miyatake et al., 1999), islets (Leibowitz et al., 1999) and muscle cells (Johnston et al., 1999). The therapeutic delivery of genes via retroviruses are currently being assessed for the treatment of various disorders such as inflammatory disease (Moldawer et al., 1999), AIDS (Amado and Chen, 1999; Engel and Kohn, 1999), cancer (Clay et al., 1999), cerebrovascular disease (Weihl et al., 1999) and hemophilia (Kay, 1998).

Herpesviral Vectors. Herpes simplex virus (HSV) type I and type II contain a double-stranded, linear DNA genome of approximately 150 kb, encoding 70-80 genes. Wild type HSV are able to infect cells lytically and to establish latency in certain cell types (e.g., neurons). Similar to adenovirus, HSV also can infect a variety of cell types including muscle (Yeung et al., 1999), ear (Derby et al., 1999), eye (Kaufman et al., 1999), tumors (Yoon et al., 1999; Howard et al., 1999), lung (Kohut et al., 1998), neuronal (Gamido et al., 1999; Lachmann and Efstathiou, 1999), liver (Miytake et al., 1999; Kooby et al., 1999) and pancreatic islets (Rabinovitch et al., 1999).

HSV viral genes are transcribed by cellular RNA polymerase II and are temporally regulated, resulting in the transcription and subsequent synthesis of gene products in roughly three discernable phases or kinetic classes. These phases of genes are referred to as the Immediate Early (IE) or α genes, Early (E) or β genes and Late (L) or γ genes. Immediately following the arrival of the genome of a virus in the nucleus of a newly infected cell, the IE genes are transcribed. The efficient expression of these genes does not require prior viral protein synthesis. The products of IE genes are required to activate transcription and regulate the remainder of the viral genome.

For use in therapeutic gene delivery, HSV must be rendered replication-defective. Protocols for generating replication-defective HSV helper virus-free cell lines have been described (U.S. Pat. No. 5,879,934; U.S. Pat. No. 5,851,826, each specifically incorporated herein by reference in its entirety). One IE protein, ICP4, also known as α4 or Vmw175, is absolutely required for both virus infectivity and the transition from IE to later transcription. Thus, due to its complex, multifunctional nature and central role in the regulation of HSV gene expression, ICP4 has typically been the target of HSV genetic studies.

Phenotypic studies of HSV viruses deleted of ICP4 indicate that such viruses will be potentially useful for gene transfer purposes (Krisky et al., 1998a). One property of viruses deleted for ICP4 that makes them desirable for gene transfer is that they only express the five other IE genes: ICP0, ICP6, ICP27, ICP22 and ICP47 (DeLuca et al., 1985), without the expression of viral genes encoding proteins that direct viral DNA synthesis, as well as the structural proteins of the virus. This property is desirable for minimizing possible deleterious effects on host cell metabolism or an immune response following gene transfer. Further deletion of IE genes ICP22 and ICP27, in addition to ICP4, substantially improve reduction of HSV cytotoxicity and prevented early and late viral gene expression (Krisky et al., 1998b).

The therapeutic potential of HSV in gene transfer has been demonstrated in various in vitro model systems and in vivo for diseases such as Parkinson's (Yamada et al., 1999), retinoblastoma (Hayashi et al., 1999), intracerebral and intradermal tumors (Moriuchi et al., 1998), B-cell malignancies (Suzuki et al., 1998), ovarian cancer (Wang et al., 1998) and Duchenne muscular dystrophy (Huard et al., 1997).

Adeno-Associated Viral Vectors. Adeno-associated virus (AAV), a member of the parvovirus family, is a human virus that is increasingly being used for gene delivery therapeutics. AAV has several advantageous features not found in other viral systems. First, AAV can infect a wide range of host cells, including non-dividing cells. Second, AAV can infect cells from different species. Third, AAV has not been associated with any human or animal disease and does not appear to alter the biological properties of the host cell upon integration. For example, it is estimated that 80-85% of the human population has been exposed to AAV. Finally, AAV is stable at a wide range of physical and chemical conditions which lends itself to production, storage and transportation requirements.

The AAV genome is a linear, single-stranded DNA molecule containing 4681 nucleotides. The AAV genome generally comprises an internal non-repeating genome flanked on each end by inverted terminal repeats (ITRs) of approximately 145 bp in length. The ITRs have multiple functions, including origins of DNA replication, and as packaging signals for the viral genome. The internal non-repeated portion of the genome includes two large open reading frames, known as the AAV replication (rep) and capsid (cap) genes. The rep and cap genes code for viral proteins that allow the virus to replicate and package the viral genome into a virion. A family of at least four viral proteins is expressed from the AAV rep region, Rep 78, Rep 68, Rep 52, and Rep 40, named according to their apparent molecular weight. The AAV cap region encodes at least three proteins, VP1, VP2, and VP3.

AAV is a helper-dependent virus requiring co-infection with a helper virus (e.g., adenovirus, herpesvirus or vaccinia) in order to form AAV virions. In the absence of co-infection with a helper virus, AAV establishes a latent state in which the viral genome inserts into a host cell chromosome, but infectious virions are not produced. Subsequent infection by a helper virus “rescues” the integrated genome, allowing it to replicate and package its genome into infectious AAV virions. Although AAV can infect cells from different species, the helper virus must be of the same species as the host cell (e.g., human AAV will replicate in canine cells co-infected with a canine adenovirus).

AAV has been engineered to deliver genes of interest by deleting the internal non-repeating portion of the AAV genome and inserting a heterologous gene between the ITRs. The heterologous gene may be functionally linked to a heterologous promoter (constitutive, cell-specific, or inducible) capable of driving gene expression in target cells. To produce infectious recombinant AAV (rAAV) containing a heterologous gene, a suitable producer cell line is transfected with a rAAV vector containing a heterologous gene. The producer cell is concurrently transfected with a second plasmid harboring the AAV rep and cap genes under the control of their respective endogenous promoters or heterologous promoters. Finally, the producer cell is infected with a helper virus.

Once these factors come together, the heterologous gene is replicated and packaged as though it were a wild-type AAV genome. When target cells are infected with the resulting rAAV virions, the heterologous gene enters and is expressed in the target cells. Because the target cells lack the rep and cap genes and the adenovirus helper genes, the rAAV cannot further replicate, package or form wild-type AAV.

The use of helper virus, however, presents a number of problems. First, the use of adenovirus in a rAAV production system causes the host cells to produce both rAAV and infectious adenovirus. The contaminating infectious adenovirus can be inactivated by heat treatment (56° C. for 1 hour). Heat treatment, however, results in approximately a 50% drop in the titer of functional rAAV virions. Second, varying amounts of adenovirus proteins are present in these preparations. For example, approximately 50% or greater of the total protein obtained in such rAAV virion preparations is free adenovirus fiber protein. If not completely removed, these adenovirus proteins have the potential of eliciting an immune response from the patient. Third, AAV vector production methods which employ a helper virus require the use and manipulation of large amounts of high titer infectious helper virus, which presents a number of health and safety concerns, particularly in regard to the use of a herpesvirus. Fourth, concomitant production of helper virus particles in rAAV virion producing cells diverts large amounts of host cellular resources away from rAAV virion production, potentially resulting in lower rAAV virion yields.

Lentiviral Vectors. Lentiviruses are complex retroviruses, which, in addition to the common retroviral genes gag, pol, and env, contain other genes with regulatory or structural function. The higher complexity enables the virus to modulate its life cycle, as in the course of latent infection. Some examples of lentivirus include the Human Immunodeficiency Viruses: HIV-1, HIV-2 and the Simian Immunodeficiency Virus: SIV. Lentiviral vectors have been generated by multiply attenuating the HIV virulence genes, for example, the genes env, vif, vpr, vpu and nef are deleted making the vector biologically safe.

Recombinant lentiviral vectors are capable of infecting non-dividing cells and can be used for both in vivo and ex vivo gene transfer and expression of nucleic acid sequences. The lentiviral genome and the proviral DNA have the three genes found in retroviruses: gag, pol and env, which are flanked by two long terminal repeat (LTR) sequences. The gag gene encodes the internal structural (matrix, capsid and nucleocapsid) proteins; the pol gene encodes the RNA-directed DNA polymerase (reverse transcriptase), a protease and an integrase; and the env gene encodes viral envelope glycoproteins. The 5′ and 3‘LTR’s serve to promote transcription and polyadenylation of the virion RNA's. The LTR contains all other cis-acting sequences necessary for viral replication. Lentiviruses have additional genes including vif, vpr, tat, rev, vpu, nef and vpx.

Adjacent to the 5′ LTR are sequences necessary for reverse transcription of the genome (the tRNA primer binding site) and for efficient encapsidation of viral RNA into particles (the Psi site). If the sequences necessary for encapsidation (or packaging of retroviral RNA into infectious virions) are missing from the viral genome, the cis defect prevents encapsidation of genomic RNA. However, the resulting mutant remains capable of directing the synthesis of all virion proteins.

Lentiviral vectors are known in the art, see Naldini et al., (1996); Zufferey et al., (1997); U.S. Pat. Nos. 6,013,516; and 5,994,136. In general, the vectors are plasmid-based or virus-based, and are configured to carry the essential sequences for incorporating foreign nucleic acid, for selection and for transfer of the nucleic acid into a host cell. The gag, pol and env genes of the vectors of interest also are known in the art. Thus, the relevant genes are cloned into the selected vector and then used to transform the target cell of interest.

Recombinant lentivirus capable of infecting a non-dividing cell wherein a suitable host cell is transfected with two or more vectors carrying the packaging functions, namely gag, pol and env, as well as rev and tat is described in U.S. Pat. No. 5,994,136, incorporated herein by reference. This describes a first vector that can provide a nucleic acid encoding a viral gag and a pol gene and another vector that can provide a nucleic acid encoding a viral env to produce a packaging cell. Introducing a vector providing a heterologous gene, such as the STAT-1a gene in this invention, into that packaging cell yields a producer cell which releases infectious viral particles carrying the foreign gene of interest. The env preferably is an amphotropic envelope protein which allows transduction of cells of human and other species.

One may target the recombinant virus by linkage of the envelope protein with an antibody or a particular ligand for targeting to a receptor of a particular cell-type. By inserting a sequence (including a regulatory region) of interest into the viral vector, along with another gene which encodes the ligand for a receptor on a specific target cell, for example, the vector is now target-specific.

The vector providing the viral env nucleic acid sequence is associated operably with regulatory sequences, e.g., a promoter or enhancer. The regulatory sequence can be any eukaryotic promoter or enhancer, including for example, the Moloney murine leukemia virus promoter-enhancer element, the human cytomegalovirus enhancer or the vaccinia P7.5 promoter. In some cases, such as the Moloney murine leukemia virus promoter-enhancer element, the promoter-enhancer elements are located within or adjacent to the LTR sequences.

The heterologous or foreign nucleic acid sequence, such as the STAT-1α encoding polynucleotide sequence herein, is linked operably to a regulatory nucleic acid sequence. Preferably, the heterologous sequence is linked to a promoter, resulting in a chimeric gene. The heterologous nucleic acid sequence may also be under control of either the viral LTR promoter-enhancer signals or of an internal promoter, and retained signals within the retroviral LTR can still bring about efficient expression of the transgene. Marker genes may be utilized to assay for the presence of the vector, and thus, to confirm infection and integration. The presence of a marker gene ensures the selection and growth of only those host cells which express the inserts. Typical selection genes encode proteins that confer resistance to antibiotics and other toxic substances, e.g., histidinol, puromycin, hygromycin, neomycin, methotrexate, etc., and cell surface markers.

The vectors are introduced via transfection or infection into the packaging cell line. The packaging cell line produces viral particles that contain the vector genome. Methods for transfection or infection are well known by those of skill in the art. After cotransfection of the packaging vectors and the transfer vector to the packaging cell line, the recombinant virus is recovered from the culture media and titered by standard methods used by those of skill in the art. Thus, the packaging constructs can be introduced into human cell lines by calcium phosphate transfection, lipofection or electroporation, generally together with a dominant selectable marker, such as neo, DHFR, Gln synthetase or ADA, followed by selection in the presence of the appropriate drug and isolation of clones. The selectable marker gene can be linked physically to the packaging genes in the construct.

Lentiviral transfer vectors Naldini et al. (1996), have been used to infect human cells growth-arrested in vitro and to transduce neurons after direct injection into the brain of adult rats. The vector was efficient at transferring marker genes in vivo into the neurons and long term expression in the absence of detectable pathology was achieved. Animals analyzed ten months after a single injection of the vector showed no decrease in the average level of transgene expression and no sign of tissue pathology or immune reaction (Blomer et al., 1997). Thus, in the present invention, one may graft or transplant cells infected with the recombinant lentivirus ex vivo, or infect cells in vivo.

Other Viral Vectors. The development and utility of viral vectors for gene delivery is constantly improving and evolving. Other viral vectors such as poxvirus; e.g., vaccinia virus (Gnant et al., 1999; Gnant et al., 1999), alpha virus; e.g., sindbis virus, Semliki forest virus (Lundstrom, 1999), reovirus (Coffey et al., 1998) and influenza A virus (Neumann et al., 1999) are contemplated for use in the present invention and may be selected according to the requisite properties of the target system.

In certain embodiments, vaccinia viral vectors are contemplated for use in the present invention. Vaccinia virus is a particularly useful eukaryotic viral vector system for expressing heterologous genes. For example, when recombinant vaccinia virus is properly engineered, the proteins are synthesized, processed and transported to the plasma membrane. Vaccinia viruses as gene delivery vectors have recently been demonstrated to transfer genes to human tumor cells, e.g., EMAP-II (Gnant et al., 1999), inner ear (Derby et al., 1999), glioma cells, e.g., p 53 (Timiryasova et al., 1999) and various mammalian cells, e.g., P₄₅₀ (U.S. Pat. No. 5,506,138). The preparation, growth and manipulation of vaccinia viruses are described in U.S. Pat. No. 5,849,304 and U.S. Pat. No. 5,506,138 (each specifically incorporated herein by reference).

In other embodiments, sindbis viral vectors are contemplated for use in gene delivery. Sindbis virus is a species of the alphavirus genus (Garoff and Li, 1998) which includes such important pathogens as Venezuelan, Western and Eastern equine encephalitis viruses (Sawai et al., 1999; Mastrangelo et al., 1999). In vitro, sindbis virus infects a variety of avian, mammalian, reptilian, and amphibian cells. The genome of sindbis virus consists of a single molecule of single-stranded RNA, 11,703 nucleotides in length. The genomic RNA is infectious, is capped at the 5′ terminus and polyadenylated at the 3′ terminus, and serves as mRNA. Translation of a vaccinia virus 26S mRNA produces a polyprotein that is cleaved co- and post-translationally by a combination of viral and presumably host-encoded proteases to give the three virus structural proteins, a capsid protein (C) and the two envelope glycoproteins (E1 and PE2, precursors of the virion E2).

Three features of sindbis virus suggest that it would be a useful vector for the expression of heterologous genes. First, its wide host range, both in nature and in the laboratory. Second, gene expression occurs in the cytoplasm of the host cell and is rapid and efficient. Third, temperature-sensitive mutations in RNA synthesis are available that may be used to modulate the expression of heterologous coding sequences by simply shifting cultures to the non-permissive temperature at various time after infection. The growth and maintenance of sindbis virus is known in the art (U.S. Pat. No. 5,217,879, specifically incorporated herein by reference).

Chimeric Viral Vectors. Chimeric or hybrid viral vectors are being developed for use in therapeutic gene delivery and are contemplated for use in the present invention. Chimeric poxviral/retroviral vectors (Holzer et al., 1999), adenoviral/retroviral vectors (Feng et al., 1997; Bilbao et al., 1997; Caplen et al., 1999) and adenoviral/adeno-associated viral vectors (Fisher et al., 1996; U.S. Pat. No. 5,871,982) have been described.

These “chimeric” viral gene transfer systems can exploit the favorable features of two or more parent viral species. For example, Wilson et al., provide a chimeric vector construct which comprises a portion of an adenovirus, AAV 5′ and 3′ ITR sequences and a selected transgene, described below (U.S. Pat. No. 5,871,983, specifically incorporate herein by reference).

The adenovirus/AAV chimeric virus uses adenovirus nucleic acid sequences as a shuttle to deliver a recombinant AAV/transgene genome to a target cell. The adenovirus nucleic acid sequences employed in the hybrid vector can range from a minimum sequence amount, which requires the use of a helper virus to produce the hybrid virus particle, to only selected deletions of adenovirus genes, which deleted gene products can be supplied in the hybrid viral production process by a selected packaging cell. At a minimum, the adenovirus nucleic acid sequences employed in the pAdA shuttle vector are adenovirus genomic sequences from which all viral genes are deleted and which contain only those adenovirus sequences required for packaging adenoviral genomic DNA into a preformed capsid head. More specifically, the adenovirus sequences employed are the cis-acting 5′ and 3′ inverted terminal repeat (ITR) sequences of an adenovirus (which function as origins of replication) and the native 5′ packaging/enhancer domain, that contains sequences necessary for packaging linear Ad genomes and enhancer elements for the E1 promoter. The adenovirus sequences may be modified to contain desired deletions, substitutions, or mutations, provided that the desired function is not eliminated.

The AAV sequences useful in the above chimeric vector are the viral sequences from which the rep and cap polypeptide encoding sequences are deleted. More specifically, the AAV sequences employed are the cis-acting 5′ and 3′ inverted terminal repeat (ITR) sequences. These chimeras are characterized by high titer transgene delivery to a host cell and the ability to stably integrate the transgene into the host cell chromosome (U.S. Pat. No. 5,871,983, specifically incorporate herein by reference). In the hybrid vector construct, the AAV sequences are flanked by the selected adenovirus sequences discussed above. The 5′ and 3′ AAV ITR sequences themselves flank a selected transgene sequence and associated regulatory elements, described below. Thus, the sequence formed by the transgene and flanking 5′ and 3′ AAV sequences may be inserted at any deletion site in the adenovirus sequences of the vector. For example, the AAV sequences are desirably inserted at the site of the deleted E1a/E1b genes of the adenovirus. Alternatively, the AAV sequences may be inserted at an E3 deletion, E2a deletion, and so on. If only the adenovirus 5′ ITR/packaging sequences and 3′ ITR sequences are used in the hybrid virus, the AAV sequences are inserted between them.

The transgene sequence of the vector and recombinant virus can be a gene, a nucleic acid sequence or reverse transcript thereof, heterologous to the adenovirus sequence, which encodes a protein, polypeptide or peptide fragment of interest. The transgene is operatively linked to regulatory components in a manner which permits transgene transcription. The composition of the transgene sequence will depend upon the use to which the resulting hybrid vector will be put. For example, one type of transgene sequence includes a therapeutic gene which expresses a desired gene product in a host cell. These therapeutic genes or nucleic acid sequences typically encode products for administration and expression in a patient in vivo or ex vivo to replace or correct an inherited or non-inherited genetic defect or treat an epigenetic disorder or disease.

(x) Non-Viral Transformation

Suitable methods for nucleic acid delivery for transformation of an organelle, a cell, a tissue or an organism for use with the current invention are believed to include virtually any method by which a nucleic acid (e.g., DNA) can be introduced into an organelle, a cell, a tissue or an organism, as described herein or as would be known to one of ordinary skill in the art. Such methods include, but are not limited to, direct delivery of DNA such as by injection (U.S. Pat. Nos. 5,994,624, 5,981,274, 5,945,100, 5,780,448, 5,736,524, 5,702,932, 5,656,610, 5,589,466 and 5,580,859, each incorporated herein by reference), including microinjection (Harland and Weintraub, 1985; U.S. Pat. No. 5,789,215, incorporated herein by reference); by electroporation (U.S. Pat. No. 5,384,253, incorporated herein by reference); by calcium phosphate precipitation (Graham and Van Der Eb, 1973; Chen and Okayama, 1987; Rippe et al., 1990); by using DEAE-dextran followed by polyethylene glycol (Gopal, 1985); by direct sonic loading (Fechheimer et al., 1987); by liposome mediated transfection (Nicolau and Sene, 1982; Fraley et al., 1979; Nicolau et al., 1987; Wong et al., 1980; Kaneda et al., 1989; Kato et al., 1991); by microprojectile bombardment (PCT Application Nos. WO 94/09699 and 95/06128; U.S. Pat. Nos. 5,610,042; 5,322,783, 5,563,055, 5,550,318, 5,538,877 and 5,538,880, and each incorporated herein by reference); by agitation with silicon carbide fibers (Kaeppler et al., 1990; U.S. Pat. Nos. 5,302,523 and 5,464,765, each incorporated herein by reference); or by PEG-mediated transformation of protoplasts (Omirulleh et al., 1993; U.S. Pat. Nos. 4,684,611 and 4,952,500, each incorporated herein by reference); by desiccation/inhibition-mediated DNA uptake (Potrykus et al., 1985). Through the application of techniques such as these, organelle(s), cell(s), tissue(s) or organism(s) may be stably or transiently transformed.

Injection. In certain embodiments, a nucleic acid may be delivered to an organelle, a cell, a tissue or an organism via one or more injections (i.e., a needle injection), such as, for example, either subcutaneously, intradermally, intramuscularly, intervenously or intraperitoneally. Methods of injection of vaccines are well known to those of ordinary skill in the art (e.g., injection of a composition comprising a saline solution). Further embodiments of the present invention include the introduction of a nucleic acid by direct microinjection. Direct microinjection has been used to introduce nucleic acid constructs into Xenopus oocytes (Harland and Weintraub, 1985).

Electroporation. In certain embodiments of the present invention, a nucleic acid is introduced into an organelle, a cell, a tissue or an organism via electroporation. Electroporation involves the exposure of a suspension of cells and DNA to a high-voltage electric discharge. In some variants of this method, certain cell wall-degrading enzymes, such as pectin-degrading enzymes, are employed to render the target recipient cells more susceptible to transformation by electroporation than untreated cells (U.S. Pat. No. 5,384,253, incorporated herein by reference). Alternatively, recipient cells can be made more susceptible to transformation by mechanical wounding.

Transfection of eukaryotic cells using electroporation has been quite successful. Mouse pre-B lymphocytes have been transfected with human κ-immunoglobulin genes (Potter et al., 1984), and rat hepatocytes have been transfected with the chloramphenicol acetyltransferase gene (Tur-Kaspa et al., 1986) in this manner.

To effect transformation by electroporation in cells such as, for example, plant cells, one may employ either friable tissues, such as a suspension culture of cells or embryogenic callus or alternatively one may transform immature embryos or other organized tissue directly. In this technique, one would partially degrade the cell walls of the chosen cells by exposing them to pectin-degrading enzymes (pectolyases) or mechanically wounding in a controlled manner. Examples of some species which have been transformed by electroporation of intact cells include maize (U.S. Pat. No. 5,384,253; Rhodes et al., 1995; D'Halluin et al., 1992), wheat (Zhou et al., 1993), tomato (Hou and Lin, 1996), soybean (Christou et al., 1987) and tobacco (Lee et al., 1989).

One also may employ protoplasts for electroporation transformation of plant cells (Bates, 1994; Lazzeri, 1995). For example, the generation of transgenic soybean plants by electroporation of cotyledon-derived protoplasts is described by Dhir and Widholm in International Patent Application No. WO 92/17598, incorporated herein by reference. Other examples of species for which protoplast transformation has been described include barley (Lazerri, 1995), sorghum (Battraw et al., 1991), maize (Bhattacharjee et al., 1997), wheat (He et al., 1994) and tomato (Tsukada, 1989).

Calcium Phosphate. In other embodiments of the present invention, a nucleic acid is introduced to the cells using calcium phosphate precipitation. Human KB cells have been transfected with adenovirus 5 DNA (Graham and Van Der Eb, 1973) using this technique. Also in this manner, mouse L (A9), mouse C127, CHO, CV-1, BHK, NIH3T3 and HeLa cells were transfected with a neomycin marker gene (Chen and Okayama, 1987), and rat hepatocytes were transfected with a variety of marker genes (Rippe et al., 1990).

DEAE-Dextran: In another embodiment, a nucleic acid is delivered into a cell using DEAE-dextran followed by polyethylene glycol. In this manner, reporter plasmids were introduced into mouse myeloma and erythroleukemia cells (Gopal, 1985).

Sonication Loading. Additional embodiments of the present invention include the introduction of a nucleic acid by direct sonic loading. LTK⁻ fibroblasts have been transfected with the thymidine kinase gene by sonication loading (Fechheimer et al., 1987).

Liposome-Mediated Transfection. In a further embodiment of the invention, a nucleic acid may be entrapped in a lipid complex such as, for example, a liposome. Liposomes are vesicular structures characterized by a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh and Bachhawat, 1991). Also contemplated is an nucleic acid complexed with Lipofectamine (Gibco BRL) or Superfect (Qiagen).

Liposome-mediated nucleic acid delivery and expression of foreign DNA in vitro has been very successful (Nicolau and Sene, 1982; Fraley et al., 1979; Nicolau et al., 1987). The feasibility of liposome-mediated delivery and expression of foreign DNA in cultured chick embryo, HeLa and hepatoma cells has also been demonstrated (Wong et al., 1980).

In certain embodiments of the invention, a liposome may be complexed with a hemagglutinating virus (HVJ). This has been shown to facilitate fusion with the cell membrane and promote cell entry of liposome-encapsulated DNA (Kaneda et al., 1989). In other embodiments, a liposome may be complexed or employed in conjunction with nuclear non-histone chromosomal proteins (HMG-1) (Kato et al., 1991). In yet further embodiments, a liposome may be complexed or employed in conjunction with both HVJ and HMG-1. In other embodiments, a delivery vehicle may comprise a ligand and a liposome.

Receptor-Mediated Transfection. Still further, a nucleic acid may be delivered to a target cell via receptor-mediated delivery vehicles. These take advantage of the selective uptake of macromolecules by receptor-mediated endocytosis that will be occurring in a target cell. In view of the cell type-specific distribution of various receptors, this delivery method adds another degree of specificity to the present invention.

Certain receptor-mediated gene targeting vehicles comprise a cell receptor-specific ligand and a nucleic acid-binding agent. Others comprise a cell receptor-specific ligand to which the nucleic acid to be delivered has been operatively attached. Several ligands have been used for receptor-mediated gene transfer (Wu and Wu, 1987; Wagner et al., 1990; Perales et al., 1994; Myers, EPO 0273085), which establishes the operability of the technique. Specific delivery in the context of another mammalian cell type has been described (Wu and Wu, 1993; incorporated herein by reference). In certain aspects of the present invention, a ligand will be chosen to correspond to a receptor specifically expressed on the target cell population.

In other embodiments, a nucleic acid delivery vehicle component of a cell-specific nucleic acid targeting vehicle may comprise a specific binding ligand in combination with a liposome. The nucleic acid(s) to be delivered are housed within the liposome and the specific binding ligand is functionally incorporated into the liposome membrane. The liposome will thus specifically bind to the receptor(s) of a target cell and deliver the contents to a cell. Such systems have been shown to be functional using systems in which, for example, epidermal growth factor (EGF) is used in the receptor-mediated delivery of a nucleic acid to cells that exhibit upregulation of the EGF receptor.

In still further embodiments, the nucleic acid delivery vehicle component of a targeted delivery vehicle may be a liposome itself, which will preferably comprise one or more lipids or glycoproteins that direct cell-specific binding. For example, lactosyl-ceramide, a galactose-terminal asialganglioside, have been incorporated into liposomes and observed an increase in the uptake of the insulin gene by hepatocytes (Nicolau et al., 1987). It is contemplated that the tissue-specific transforming constructs of the present invention can be specifically delivered into a target cell in a similar manner.

G. Expression Systems

Numerous expression systems exist that comprise at least a part or all of the compositions discussed above. Prokaryote- and/or eukaryote-based systems can be employed for use with the present invention to produce nucleic acid sequences, or their cognate polypeptides, proteins and peptides. Many such systems are commercially and widely available.

The insect cell/baculovirus system can produce a high level of protein expression of a heterologous nucleic acid segment, such as described in U.S. Pat. Nos. 5,871,986 and 4,879,236, both herein incorporated by reference, and which can be bought, for example, under the name MAxBAc® 2.0 from INVITROGEN® and BACPACK™ BACULOVIRUS EXPRESSION SYSTEM FROM CLONTECH®.

Other examples of expression systems include STRATAGENE®'s COMPLETE CONTROL™ Inducible Mammalian Expression System, which involves a synthetic ecdysone-inducible receptor, or its pET Expression System, an E. coli expression system. Another example of an inducible expression system is available from INVITROGEN®, which carries the T-REX™ (tetracycline-regulated expression) System, an inducible mammalian expression system that uses the full-length CMV promoter. INVITROGEN® also provides a yeast expression system called the Pichia methanolica Expression System, which is designed for high-level production of recombinant proteins in the methylotrophic yeast Pichia methanolica. One of skill in the art would know how to express a vector, such as an expression construct, to produce a nucleic acid sequence or its cognate polypeptide, protein, or peptide.

Primary mammalian cell cultures may be prepared in various ways. In order for the cells to be kept viable while in vitro and in contact with the expression construct, it is necessary to ensure that the cells maintain contact with the correct ratio of oxygen and carbon dioxide and nutrients but are protected from microbial contamination. Cell culture techniques are well documented.

One embodiment of the foregoing involves the use of gene transfer to immortalize cells for the production of proteins. The gene for the protein of interest may be transferred as described above into appropriate host cells followed by culture of cells under the appropriate conditions. The gene for virtually any polypeptide may be employed in this manner. The generation of recombinant expression vectors, and the elements included therein, are discussed above. Alternatively, the protein to be produced may be an endogenous protein normally synthesized by the cell in question.

Examples of useful mammalian host cell lines are Vero and HeLa cells and cell lines of Chinese hamster ovary, W138, BHK, COS-7, 293, HepG2, NIH3T3, RIN and MDCK cells. In addition, a host cell strain may be chosen that modulates the expression of the inserted sequences, or modifies and process the gene product in the manner desired. Such modifications (e.g., glycosylation) and processing (e.g., cleavage) of protein products may be important for the function of the protein. Different host cells have characteristic and specific mechanisms for the post-translational processing and modification of proteins. Appropriate cell lines or host systems can be chosen to insure the correct modification and processing of the foreign protein expressed.

A number of selection systems may be used including, but not limited to, HSV thymidine kinase, hypoxanthine-guanine phosphoribosyltransferase and adenine phosphoribosyltransferase genes, in tk-, hgprt- or aprt-cells, respectively. Also, anti-metabolite resistance can be used as the basis of selection for dhfr, that confers resistance to; gpt, that confers resistance to mycophenolic acid; neo, that confers resistance to the aminoglycoside G418; and hygro, that confers resistance to hygromycin.

### III. Antibodies Reactive With t-DARPP

In another aspect, the present invention contemplates an antibody that is immunoreactive with a t-DARPP molecule of the present invention, or any portion thereof. An antibody can be a polyclonal or a monoclonal antibody. In a particular embodiment, an antibody is a monoclonal antibody. Means for preparing and characterizing antibodies are well known in the art (see, e.g., Harlow and Lane, 1988).

Briefly, a polyclonal antibody is prepared by immunizing an animal with an immunogen comprising a polypeptide of the present invention and collecting antisera from that immunized animal. A wide range of animal species can be used for the production of antisera. Typically an animal used for production of anti-antisera is a non-human animal including rabbits, mice, rats, hamsters, pigs or horses. Because of the relatively large blood volume of rabbits, a rabbit is a preferred choice for production of polyclonal antibodies.

Antibodies, both polyclonal and monoclonal, specific for isoforms of antigen may be prepared using conventional immunization techniques, as will be generally known to those of skill in the art. A composition containing antigenic epitopes of the compounds of the present invention can be used to immunize one or more experimental animals, such as a rabbit or mouse, which will then proceed to produce specific antibodies against the compounds of the present invention. Polyclonal antisera may be obtained, after allowing time for antibody generation, simply by bleeding the animal and preparing serum samples from the whole blood.

It is proposed that the monoclonal antibodies of the present invention will find useful application in standard immunochemical procedures, such as ELISA and Western blot methods and in immunohistochemical procedures such as tissue staining, as well as in other procedures which may utilize antibodies specific to t-Darpp-related antigen epitopes. Additionally, it is proposed that monoclonal antibodies against t-Darpp may be used as therapeutics.

Means for preparing and characterizing antibodies are well known in the art (see, e.g., Harlow and Lane, 1988; incorporated herein by reference). More specific examples of monoclonal antibody preparation are given in the examples below.

As is well known in the art, a given composition may vary in its immunogenicity. It is often necessary therefore to boost the host immune system, as may be achieved by coupling a peptide or polypeptide immunogen to a carrier. Exemplary and preferred carriers are keyhole limpet hemocyanin (KLH) and bovine serum albumin (BSA). Other albumins such as ovalbumin, mouse serum albumin or rabbit serum albumin can also be used as carriers. Means for conjugating a polypeptide to a carrier protein are well known in the art and include glutaraldehyde, m-maleimidobencoyl-N-hydroxysuccinimide ester, carbodiimide and bis-biazotized benzidine.

As also is well known in the art, the immunogenicity of a particular immunogen composition can be enhanced by the use of non-specific stimulators of the immune response, known as adjuvants. Exemplary and preferred adjuvants include complete Freund's adjuvant (a non-specific stimulator of the immune response containing killed Mycobacterium tuberculosis), incomplete Freund's adjuvants and aluminum hydroxide adjuvant.

The amount of immunogen composition used in the production of polyclonal antibodies varies upon the nature of the immunogen as well as the animal used for immunization. A variety of routes can be used to administer the immunogen (subcutaneous, intramuscular, intradermal, intravenous and intraperitoneal). The production of polyclonal antibodies may be monitored by sampling blood of the immunized animal at various points following immunization. A second, booster, injection may also be given. The process of boosting and titering is repeated until a suitable titer is achieved. When a desired level of immunogenicity is obtained, the immunized animal can be bled and the serum isolated and stored, and/or the animal can be used to generate mAbs.

MAbs may be readily prepared through use of well-known techniques, such as those exemplified in U.S. Pat. No. 4,196,265, incorporated herein by reference. Typically, this technique involves immunizing a suitable animal with a selected immunogen composition, e.g., a purified or partially purified T-Darpp protein, polypeptide or peptide or cell expressing high levels of T-Darpp. The immunizing composition is administered in a manner effective to stimulate antibody producing cells. Rodents such as mice and rats are preferred animals, however, the use of rabbit, sheep frog cells is also possible. The use of rats may provide certain advantages (Goding, 1986), but mice are preferred, with the BALB/c mouse being most preferred as this is most routinely used and generally gives a higher percentage of stable fusions.

Following immunization, somatic cells with the potential for producing antibodies, specifically B-lymphocytes (B-cells), are selected for use in the mAb generating protocol. These cells may be obtained from biopsied spleens, tonsils or lymph nodes, or from a peripheral blood sample. Spleen cells and peripheral blood cells are preferred, the former because they are a rich source of antibody-producing cells that are in the dividing plasmablast stage, and the latter because peripheral blood is easily accessible. Often, a panel of animals will have been immunized and the spleen of animal with the highest antibody titer will be removed and the spleen lymphocytes obtained by homogenizing the spleen with a syringe. Typically, a spleen from an immunized mouse contains approximately 5×10⁷ to 2×10⁸ lymphocytes.

The antibody-producing B lymphocytes from the immunized animal are then fused with cells of an immortal myeloma cell, generally one of the same species as the animal that was immunized. Myeloma cell lines suited for use in hybridoma-producing fusion procedures preferably are non-antibody-producing, have high fusion efficiency, and enzyme deficiencies that render then incapable of growing in certain selective media which support the growth of only the desired fused cells (hybridomas).

Any one of a number of myeloma cells may be used, as are known to those of skill in the art (Goding, 1986; Campbell, 1984). For example, where the immunized animal is a mouse, one may use P3-X63/Ag8, P3-X63-Ag8.653, NS1/1.Ag 4 1, Sp210-Ag14, FO, NSO/U, MPC-11, MPC11-X45-GTG 1.7 and S194/5XX0 Bul; for rats, one may use R210.RCY3, Y3-Ag 1.2.3, IR983F and 4B210; and U-266, GM1500-GRG2, LICR-LON-HMy2 and UC729-6 are all useful in connection with cell fusions.

Methods for generating hybrids of antibody-producing spleen or lymph node cells and myeloma cells usually comprise mixing somatic cells with myeloma cells in a 2:1 ratio, though the ratio may vary from about 20:1 to about 1:1, respectively, in the presence of an agent or agents (chemical or electrical) that promote the fusion of cell membranes. Fusion methods using Sendai virus have been described (Kohler and Milstein, 1975; 1976), and those using polyethylene glycol (PEG), such as 37% (v/v) PEG, by Gefter et al., (1977). The use of electrically induced fusion methods is also appropriate (Goding, 1986).

Fusion procedures usually produce viable hybrids at low frequencies, around 1×10⁻⁶ to 1×10⁻⁸. However, this does not pose a problem, as the viable, fused hybrids are differentiated from the parental, unfused cells (particularly the unfused myeloma cells that would normally continue to divide indefinitely) by culturing in a selective medium. The selective medium is generally one that contains an agent that blocks the de novo synthesis of nucleotides in the tissue culture media. Exemplary and preferred agents are aminopterin, methotrexate, and azaserine. Aminopterin and methotrexate block de novo synthesis of both purines and pyrimidines, whereas azaserine blocks only purine synthesis. Where aminopterin or methotrexate is used, the media is supplemented with hypoxanthine and thymidine as a source of nucleotides (HAT medium). Where azaserine is used, the media is supplemented with hypoxanthine.

The preferred selection medium is HAT. Only cells capable of operating nucleotide salvage pathways are able to survive in HAT medium. The myeloma cells are defective in key enzymes of the salvage pathway, e.g., hypoxanthine phosphoribosyl transferase (HPRT), and they cannot survive. The B-cells can operate this pathway, but they have a limited life span in culture and generally die within about two weeks. Therefore, the only cells that can survive in the selective media are those hybrids formed from myeloma and B-cells.

This culturing provides a population of hybridomas from which specific hybridomas are selected. Typically, selection of hybridomas is performed by culturing the cells by single-clone dilution in microtiter plates, followed by testing the individual clonal supernatants (after about two to three weeks) for the desired reactivity. The assay should be sensitive, simple and rapid, such as radioimmunoassays, enzyme immunoassays, cytotoxicity assays, plaque assays, dot immunobinding assays, and the like.

The selected hybridomas would then be serially diluted and cloned into individual antibody-producing cell lines, which clones can then be propagated indefinitely to provide mAbs. The cell lines may be exploited for mAb production in two basic ways. A sample of the hybridoma can be injected (often into the peritoneal cavity) into a histocompatible animal of the type that was used to provide the somatic and myeloma cells for the original fusion. The injected animal develops tumors secreting the specific monoclonal antibody produced by the fused cell hybrid. The body fluids of the animal, such as serum or ascites fluid, can then be tapped to provide mAbs in high concentration. The individual cell lines could also be cultured in vitro, where the mAbs are naturally secreted into the culture medium from which they can be readily obtained in high concentrations. mAbs produced by either means may be further purified, if desired, using filtration, centrifugation and various chromatographic methods such as HPLC or affinity chromatography.

### IV. Diagnosing Cancers Involving t-DARPP

t-DARPP and the corresponding gene may be employed as a diagnostic or prognostic target for cancer. More specifically, point mutations, deletions, insertions or regulatory pertubations relating to t-DARPP may cause cancer or promote cancer development, cause or promoter tumor progression at a primary site, and/or cause or promote metastasis. In particular, elevated t-DARPP levels, as compared to comparable “normal” or non-cancer cells (e.g., cells of the same tissue origin) may occur in cancer cells. Other phenomena associated with malignancy that may be affected by t-Darpp expression include angiogenesis and tissue invasion.

A. Genetic Diagnosis

One embodiment of the instant invention comprises a method for detecting variation in the expression of t-DARPP. This may comprises determining that level of t-DARPP in a tissue or fluid sample. Obviously, this sort of assay has importance in the diagnosis of related cancers. Such cancer may involve cancers of the brain (glioblastomas, medulloblastoma, astrocytoma, oligodendroglioma, ependymomas), lung, liver, spleen, kidney, pancreas, small intestine, blood cells, lymph node, colon, breast, endometrium, stomach, prostate, testicle, ovary, skin, head and neck, esophagus, bone marrow, blood or other tissue. In particular, the present invention relates to the diagnosis of breast cancers.

The biological sample can be any tissue or fluid. Various embodiments include cells of the skin, muscle, facia, brain, prostate, breast, endometrium, lung, head & neck, pancreas, small intestine, blood cells, liver, testes, ovaries, colon, skin, stomach, esophagus, spleen, lymph node, bone marrow or kidney. Other embodiments include fluid samples such as peripheral blood, lymph fluid, ascites, serous fluid, pleural effusion, sputum, cerebrospinal fluid, lacrimal fluid, stool or urine.

Nucleic acid used is isolated from cells contained in the biological sample, according to standard methodologies (Sambrook et al., 1989). The nucleic acid may be genomic DNA or fractionated or whole cell RNA. Where RNA is used, it may be desired to convert the RNA to a complementary DNA. In one embodiment, the RNA is whole cell RNA; in another, it is poly-A RNA. Normally, the nucleic acid is amplified.

Depending on the format, the specific nucleic acid of interest is identified in the sample directly using amplification or with a second, known nucleic acid following amplification. Next, the identified product is detected. In certain applications, the detection may be performed by visual means (e.g., ethidium bromide staining of a gel). Alternatively, the detection may involve indirect identification of the product via chemiluminescence, radioactive scintigraphy of radiolabel or fluorescent label or even via a system using electrical or thermal impulse signals (Affymax Technology; Bellus, 1994).

Following detection, one may compare the results seen in a given patient with a statistically significant reference group of normal patients and patients that have t-DARPP-related cancers. In this way, it is possible to correlate the amount of t-DARPP detected with various clinical states.

(i) Primers and Probes

The term primer, as defined herein, is meant to encompass any nucleic acid that is capable of priming the synthesis of a nascent nucleic acid in a template-dependent process. Typically, primers are oligonucleotides from ten to twenty base pairs in length, but longer sequences can be employed. Primers may be provided in double-stranded or single-stranded form, although the single-stranded form is preferred. Probes are defined differently, although they may act as primers. Probes, while perhaps capable of priming, are designed to binding to the target DNA or RNA and need not be used in an amplification process. In particular embodiments, the probes or primers are labeled with radioactive species (³²P, ¹⁴C, ³⁵S, ³H, or other label), with a fluorophore (rhodamine, fluorescein) or a chemillumiscent (luciferase).

(ii) Template Dependent Amplification Methods

A number of template dependent processes are available to amplify the marker sequences present in a given template sample. One of the best known amplification methods is the polymerase chain reaction (referred to as PCR™) which is described in detail in U.S. Pat. Nos. 4,683,195, 4,683,202 and 4,800,159, and in Innis et al., 1990, each of which is incorporated herein by reference in its entirety.

Briefly, in PCR™, two primer sequences are prepared that are complementary to regions on opposite complementary strands of the marker sequence. An excess of deoxynucleoside triphosphates are added to a reaction mixture along with a DNA polymerase, e.g., Taq polymerase. If the marker sequence is present in a sample, the primers will bind to the marker and the polymerase will cause the primers to be extended along the marker sequence by adding on nucleotides. By raising and lowering the temperature of the reaction mixture, the extended primers will dissociate from the marker to form reaction products, excess primers will bind to the marker and to the reaction products and the process is repeated.

A reverse transcriptase PCR™ amplification procedure may be performed in order to quantify the amount of mRNA amplified. Methods of reverse transcribing RNA into cDNA are well known and described in Sambrook et al. (1989). Alternative methods for reverse transcription utilize thermostable, RNA-dependent DNA polymerases. These methods are described in WO 90/07641 filed Dec. 21, 1990. Polymerase chain reaction methodologies are well known in the art.

Another method for amplification is the ligase chain reaction (“LCR”), disclosed in EPO No. 320 308, incorporated herein by reference in its entirety. In LCR, two complementary probe pairs are prepared, and in the presence of the target sequence, each pair will bind to opposite complementary strands of the target such that they abut. In the presence of a ligase, the two probe pairs will link to form a single unit. By temperature cycling, as in PCR™, bound ligated units dissociate from the target and then serve as “target sequences” for ligation of excess probe pairs. U.S. Pat. No. 4,883,750 describes a method similar to LCR for binding probe pairs to a target sequence.

Qbeta Replicase, described in PCT Application No. PCT/US87/00880, may also be used as still another amplification method in the present invention. In this method, a replicative sequence of RNA that has a region complementary to that of a target is added to a sample in the presence of an RNA polymerase. The polymerase will copy the replicative sequence that can then be detected.

An isothermal amplification method, in which restriction endonucleases and ligases are used to achieve the amplification of target molecules that contain nucleotide 5′-[alpha-thio]-triphosphates in one strand of a restriction site may also be useful in the amplification of nucleic acids in the present invention (Walker et al., 1992).

Strand Displacement Amplification (SDA) is another method of carrying out isothermal amplification of nucleic acids which involves multiple rounds of strand displacement and synthesis, i.e., nick translation. A similar method, called Repair Chain Reaction (RCR), involves annealing several probes throughout a region targeted for amplification, followed by a repair reaction in which only two of the four bases are present. The other two bases can be added as biotinylated derivatives for easy detection. A similar approach is used in SDA. Target specific sequences can also be detected using a cyclic probe reaction (CPR). In CPR, a probe having 3′ and 5′ sequences of non-specific DNA and a middle sequence of specific RNA is hybridized to DNA that is present in a sample. Upon hybridization, the reaction is treated with RNase H, and the products of the probe identified as distinctive products that are released after digestion. The original template is annealed to another cycling probe and the reaction is repeated.

Still another amplification methods described in GB Application No. 2 202 328, and in PCT Application No. PCT/US89/01025, each of which is incorporated herein by reference in its entirety, may be used in accordance with the present invention. In the former application, “modified” primers are used in a PCR™-like, template- and enzyme-dependent synthesis. The primers may be modified by labeling with a capture moiety (e.g., biotin) and/or a detector moiety (e.g., enzyme). In the latter application, an excess of labeled probes are added to a sample. In the presence of the target sequence, the probe binds and is cleaved catalytically. After cleavage, the target sequence is released intact to be bound by excess probe. Cleavage of the labeled probe signals the presence of the target sequence.

Other nucleic acid amplification procedures include transcription-based amplification systems (TAS), including nucleic acid sequence based amplification (NASBA) and 3SR (Kwoh et al., 1989; Gingeras et al., PCT Application WO 88/10315, incorporated herein by reference in their entirety). In NASBA, the nucleic acids can be prepared for amplification by standard phenol/chloroform extraction, heat denaturation of a clinical sample, treatment with lysis buffer and minispin columns for isolation of DNA and RNA or guanidinium chloride extraction of RNA. These amplification techniques involve annealing a primer which has target specific sequences. Following polymerization, DNA/RNA hybrids are digested with RNase H while double stranded DNA molecules are heat denatured again. In either case the single stranded DNA is made fully double-stranded by addition of second target specific primer, followed by polymerization. The double-stranded DNA molecules are then multiply transcribed by an RNA polymerase such as T7 or SP6. In an isothermal cyclic reaction, the RNA's are reverse transcribed into single-stranded DNA, which is then converted to double stranded DNA, and then transcribed once again with an RNA polymerase such as T7 or SP6. The resulting products, whether truncated or complete, indicate target specific sequences.

Davey et al., EPO No. 329 822 (incorporated herein by reference in its entirety) disclose a nucleic acid amplification process involving cyclically synthesizing single-stranded RNA (“ssRNA”), ssDNA, and double-stranded DNA (dsDNA), which may be used in accordance with the present invention. The ssRNA is a template for a first primer oligonucleotide, which is elongated by reverse transcriptase (RNA-dependent DNA polymerase). The RNA is then removed from the resulting DNA:RNA duplex by the action of ribonuclease H(RNase H, an RNase specific for RNA in duplex with either DNA or RNA). The resultant ssDNA is a template for a second primer, which also includes the sequences of an RNA polymerase promoter (exemplified by T7 RNA polymerase) 5′ to its homology to the template. This primer is then extended by DNA polymerase (exemplified by the large “Klenow” fragment of E. coli DNA polymerase I), resulting in a double-stranded DNA (“dsDNA”) molecule, having a sequence identical to that of the original RNA between the primers and having additionally, at one end, a promoter sequence. This promoter sequence can be used by the appropriate RNA polymerase to make many RNA copies of the DNA. These copies can then re-enter the cycle leading to very swift amplification. With proper choice of enzymes, this amplification can be done isothermally without addition of enzymes at each cycle. Because of the cyclical nature of this process, the starting sequence can be chosen to be in the form of either DNA or RNA.

Miller et al., PCT Application WO 89/06700 (incorporated herein by reference in its entirety) disclose a nucleic acid sequence amplification scheme based on the hybridization of a promoter/primer sequence to a target single-stranded DNA (“ssDNA”) followed by transcription of many RNA copies of the sequence. This scheme is not cyclic, i.e., new templates are not produced from the resultant RNA transcripts. Other amplification methods include “RACE” and “one-sided PCR™” (Frohman, 1990; Ohara et al., 1989; each herein incorporated by reference in their entirety).

Methods based on ligation of two (or more) oligonucleotides in the presence of nucleic acid having the sequence of the resulting “di-oligonucleotide”, thereby amplifying the di-oligonucleotide, may also be used in the amplification step of the present invention. Wu et al., (1989), incorporated herein by reference in its entirety.

(iii) Southern/Northern Blotting

Blotting techniques are well known to those of skill in the art. Southern blotting involves the use of DNA as a target, whereas Northern blotting involves the use of RNA as a target. Each provide different types of information, although cDNA blotting is analogous, in many aspects, to blotting or RNA species.

Briefly, a probe is used to target a DNA or RNA species that has been immobilized on a suitable matrix, often a filter of nitrocellulose. The different species should be spatially separated to facilitate analysis. This often is accomplished by gel electrophoresis of nucleic acid species followed by “blotting” on to the filter.

Subsequently, the blotted target is incubated with a probe (usually labeled) under conditions that promote denaturation and rehybridization. Because the probe is designed to base pair with the target, the probe will binding a portion of the target sequence under renaturing conditions. Unbound probe is then removed, and detection is accomplished as described above.

(iv) Separation Methods

It normally is desirable, at one stage or another, to separate the amplification product from the template and the excess primer for the purpose of determining whether specific amplification has occurred. In one embodiment, amplification products are separated by agarose, agarose-acrylamide or polyacrylamide gel electrophoresis using standard methods. See Sambrook et al., 1989.

Alternatively, chromatographic techniques may be employed to effect separation. There are many kinds of chromatography which may be used in the present invention: adsorption, partition, ion-exchange and molecular sieve, and many specialized techniques for using them including column, paper, thin-layer and gas chromatography (Freifelder, 1982).

(v) Detection Methods

Products may be visualized in order to confirm amplification of the marker sequences. One typical visualization method involves staining of a gel with ethidium bromide and visualization under UV light. Alternatively, if the amplification products are integrally labeled with radio- or fluorometrically-labeled nucleotides, the amplification products can then be exposed to x-ray film or visualized under the appropriate stimulating spectra, following separation.

In one embodiment, visualization is achieved indirectly. Following separation of amplification products, a labeled nucleic acid probe is brought into contact with the amplified marker sequence. The probe preferably is conjugated to a chromophore but may be radiolabeled. In another embodiment, the probe is conjugated to a binding partner, such as an antibody or biotin, and the other member of the binding pair carries a detectable moiety.

In one embodiment, detection is by a labeled probe. The techniques involved are well known to those of skill in the art and can be found in many standard books on molecular protocols. See Sambrook et al., 1989. For example, chromophore or radiolabel probes or primers identify the target during or following amplification.

One example of the foregoing is described in U.S. Pat. No. 5,279,721, incorporated by reference herein, which discloses an apparatus and method for the automated electrophoresis and transfer of nucleic acids. The apparatus permits electrophoresis and blotting without external manipulation of the gel and is ideally suited to carrying out methods according to the present invention.

In addition, the amplification products described above may be subjected to sequence analysis to identify specific kinds of variations using standard sequence analysis techniques. Within certain methods, exhaustive analysis of genes is carried out by sequence analysis using primer sets designed for optimal sequencing (Pignon et al, 1994). The present invention provides methods by which any or all of these types of analyses may be used. Using the sequences disclosed herein, oligonucleotide primers may be designed to permit the amplification of sequences throughout the T-Darpp gene that may then be analyzed by direct sequencing.

(vi) Kit Components

All the essential materials and reagents required for detecting and sequencing t-DARPP and variants thereof may be assembled together in a kit. This generally will comprise preselected primers and probes. Also included may be enzymes suitable for amplifying nucleic acids including various polymerases (RT, Taq, Sequenase™ etc.), deoxynucleotides and buffers to provide the necessary reaction mixture for amplification. Such kits also generally will comprise, in suitable means, distinct containers for each individual reagent and enzyme as well as for each primer or probe.

(vii) Design and Theoretical Considerations for Relative Quantitative RT-PCR™

Reverse transcription (RT) of RNA to cDNA followed by relative quantitative PCR™ (RT-PCR™) can be used to determine the relative concentrations of specific mRNA species isolated from patients. By determining that the concentration of a specific mRNA species varies, it is shown that the gene encoding the specific mRNA species is differentially expressed.

In PCR™, the number of molecules of the amplified target DNA increase by a factor approaching two with every cycle of the reaction until some reagent becomes limiting. Thereafter, the rate of amplification becomes increasingly diminished until there is no increase in the amplified target between cycles. If a graph is plotted in which the cycle number is on the X axis and the log of the concentration of the amplified target DNA is on the Y axis, a curved line of characteristic shape is formed by connecting the plotted points. Beginning with the first cycle, the slope of the line is positive and constant. This is said to be the linear portion of the curve. After a reagent becomes limiting, the slope of the line begins to decrease and eventually becomes zero. At this point the concentration of the amplified target DNA becomes asymptotic to some fixed value. This is said to be the plateau portion of the curve.

The concentration of the target DNA in the linear portion of the PCR™ amplification is directly proportional to the starting concentration of the target before the reaction began. By determining the concentration of the amplified products of the target DNA in PCR™ reactions that have completed the same number of cycles and are in their linear ranges, it is possible to determine the relative concentrations of the specific target sequence in the original DNA mixture. If the DNA mixtures are cDNAs synthesized from RNAs isolated from different tissues or cells, the relative abundances of the specific mRNA from which the target sequence was derived can be determined for the respective tissues or cells. This direct proportionality between the concentration of the PCR™ products and the relative mRNA abundances is only true in the linear range of the PCR™ reaction.

The final concentration of the target DNA in the plateau portion of the curve is determined by the availability of reagents in the reaction mix and is independent of the original concentration of target DNA. Therefore, the first condition that must be met before the relative abundances of a mRNA species can be determined by RT-PCR™ for a collection of RNA populations is that the concentrations of the amplified PCR™ products must be sampled when the PCR™ reactions are in the linear portion of their curves.

The second condition that must be met for an RT-PCR™ experiment to successfully determine the relative abundances of a particular mRNA species is that relative concentrations of the amplifiable cDNAs must be normalized to some independent standard. The goal of an RT-PCR™ experiment is to determine the abundance of a particular mRNA species relative to the average abundance of all mRNA species in the sample. In the experiments described below, mRNAs for β-actin, asparagine synthetase and lipocortin II were used as external and internal standards to which the relative abundance of other mRNAs are compared.

Most protocols for competitive PCR™ utilize internal PCR™ standards that are approximately as abundant as the target. These strategies are effective if the products of the PCR™ amplifications are sampled during their linear phases. If the products are sampled when the reactions are approaching the plateau phase, then the less abundant product becomes relatively over represented. Comparisons of relative abundances made for many different RNA samples, such as is the case when examining RNA samples for differential expression, become distorted in such a way as to make differences in relative abundances of RNAs appear less than they actually are. This is not a significant problem if the internal standard is much more abundant than the target. If the internal standard is more abundant than the target, then direct linear comparisons can be made between RNA samples.

The above discussion describes theoretical considerations for an RT-PCR™ assay for clinically derived materials. The problems inherent in clinical samples are that they are of variable quantity (making normalization problematic), and that they are of variable quality (necessitating the co-amplification of a reliable internal control, preferably of larger size than the target). Both of these problems are overcome if the RT-PCR™ is performed as a relative quantitative RT-PCR™ with an internal standard in which the internal standard is an amplifiable cDNA fragment that is larger than the target cDNA fragment and in which the abundance of the mRNA encoding the internal standard is roughly 5-100 fold higher than the mRNA encoding the target. This assay measures relative abundance, not absolute abundance of the respective mRNA species.

Other studies may be performed using a more conventional relative quantitative RT-PCR™ assay with an external standard protocol. These assays sample the PCR™ products in the linear portion of their amplification curves. The number of PCR™ cycles that are optimal for sampling must be empirically determined for each target cDNA fragment. In addition, the reverse transcriptase products of each RNA population isolated from the various tissue samples must be carefully normalized for equal concentrations of amplifiable cDNAs. This consideration is very important since the assay measures absolute mRNA abundance. Absolute mRNA abundance can be used as a measure of differential gene expression only in normalized samples. While empirical determination of the linear range of the amplification curve and normalization of cDNA preparations are tedious and time consuming processes, the resulting RT-PCR™ assays can be superior to those derived from the relative quantitative RT-PCR™ assay with an internal standard.

One reason for this advantage is that without the internal standard/competitor, all of the reagents can be converted into a single PCR™ product in the linear range of the amplification curve, thus increasing the sensitivity of the assay. Another reason is that with only one PCR™ product, display of the product on an electrophoretic gel or another display method becomes less complex, has less background and is easier to interpret.

(viii) Chip Technologies

Specifically contemplated by the present inventors are chip-based DNA technologies such as those described by Hacia et al. (1996) and Shoemaker et al. (1996). Briefly, these techniques involve quantitative methods for analyzing large numbers of genes rapidly and accurately. By tagging genes with oligonucleotides or using fixed probe arrays, one can employ chip technology to segregate target molecules as high density arrays and screen these molecules on the basis of hybridization. See also Pease et al. (1994); Fodor et al. (1991).

B. Immunodiagnosis

Antibodies of the present invention can be used in characterizing the t-DARPP content of healthy and diseased tissues, through techniques such as ELISAs and Western blotting. This may provide a screen for the presence or absence of malignancy or as a predictor of future cancer. To distinguish t-DARPP from DARPP-32, one can separate the two antigens on the basis of size, or an antibody that binds to one but not the other (e.g., binding in the portion encoded by the alternative first exons) can be utilized.

The use of antibodies of the present invention, in an ELISA assay is contemplated. For example, anti-t-DARPP antibodies are immobilized onto a selected surface, preferably a surface exhibiting a protein affinity such as the wells of a polystyrene microtiter plate. After washing to remove incompletely adsorbed material, it is desirable to bind or coat the assay plate wells with a non-specific protein that is known to be antigenically neutral with regard to the test antisera such as bovine serum albumin (BSA), casein or solutions of powdered milk. This allows for blocking of non-specific adsorption sites on the immobilizing surface and thus reduces the background caused by non-specific binding of antigen onto the surface.

After binding of antibody to the well, coating with a non-reactive material to reduce background, and washing to remove unbound material, the immobilizing surface is contacted with the sample to be tested in a manner conducive to immune complex (antigen/antibody) formation.

Following formation of specific immunocomplexes between the test sample and the bound antibody, and subsequent washing, the occurrence and even amount of immunocomplex formation may be determined by subjecting same to a second antibody having specificity for t-DARPP that differs from the first antibody. Appropriate conditions preferably include diluting the sample with diluents such as BSA, bovine gamma globulin (BGG) and phosphate buffered saline (PBS)/Tween®. These added agents also tend to assist in the reduction of nonspecific background. The layered antisera are then allowed to incubate for from about 2 to about 4 hr, at temperatures preferably on the order of about 25° to about 27° C. Following incubation, the antisera-contacted surface is washed so as to remove non-immunocomplexed material. A preferred washing procedure includes washing with a solution such as PBS/Tween®, or borate buffer.

To provide a detecting means, the second antibody will preferably have an associated enzyme that will generate a color development upon incubating with an appropriate chromogenic substrate. Thus, for example, one will desire to contact and incubate the second antibody-bound surface with a urease or peroxidase-conjugated anti-human IgG for a period of time and under conditions which favor the development of immunocomplex formation (e.g., incubation for 2 hr at room temperature in a PBS-containing solution such as PBS/Tween®).

After incubation with the second enzyme-tagged antibody, and subsequent to washing to remove unbound material, the amount of label is quantified by incubation with a chromogenic substrate such as urea and bromocresol purple or 2,2′-azino-di-(3-ethyl-benzthiazoline)-6-sulfonic acid (ABTS) and H₂O₂, in the case of peroxidase as the enzyme label. Quantitation is then achieved by measuring the degree of color generation, e.g., using a visible spectrum spectrophotometer.

The preceding format may be altered by first binding the sample to the assay plate. Then, primary antibody is incubated with the assay plate, followed by detecting of bound primary antibody using a labeled second antibody with specificity for the primary antibody.

The antibody compositions of the present invention will find great use in immunoblot or Western blot analysis. The antibodies may be used as high-affinity primary reagents for the identification of proteins immobilized onto a solid support matrix, such as nitrocellulose, nylon or combinations thereof. In conjunction with immunoprecipitation, followed by gel electrophoresis, these may be used as a single step reagent for use in detecting antigens against which secondary reagents used in the detection of the antigen cause an adverse background. Immunologically-based detection methods for use in conjunction with Western blotting include enzymatically-, radiolabel-, or fluorescently-tagged secondary antibodies against the toxin moiety are considered to be of particular use in this regard.

### V. Methods of Therapy

The present invention also involves, in another embodiment, the treatment of cancer. The types of cancer that may be treated, according to the present invention, is limited only by the involvement of t-DARPP. Thus, it is contemplated that a wide variety of tumors may be treated using t-DARPP-targeted therapy, including cancers of the brain, lung, liver, spleen, kidney, lymph node, pancreas, small intestine, blood cells, colon, stomach, breast, endometrium, prostate, testicle, ovary, skin, head and neck, esophagus, bone marrow, blood or other tissue.

In many contexts, it is not necessary that the tumor cell be killed or induced to undergo normal cell death or “apoptosis.” Rather, to accomplish a meaningful treatment, all that is required is that the tumor growth be slowed to some degree. It may be that the tumor growth is completely blocked, however, or that some tumor regression is achieved. Clinical terminology such as “remission” and “reduction of tumor” burden also are contemplated given their normal usage.

The present invention contemplates the use of t-DARPP inhibitors as a single-agent therapy against cancers that exhibit elevated t-DARPP activity, due to overexpression, reduced turnover, and/or mutant forms of t-DARPP. It may also be used in combination with one or more additional anti-cancer therapies, such as radio-, chemo, immune, hormonal, or toxin therapy. However, in particular, the invention is designed to function in combination with HER2/Neu targeting agents.

A. Genetic Based Therapies

One of the therapeutic embodiments contemplated by the present inventors is the intervention, at the molecular level, in the events involved in tumorigenesis. Specifically, the present inventors intend to provide, to a cancer cell, an expression construct capable antagonizing t-DARPP function in that cell. The lengthy discussion of expression vectors and the genetic elements employed therein is incorporated into this section by reference. Particular expression vectors are viral vectors such as adenovirus, adeno-associated virus, herpesvirus, vaccinia virus and retrovirus. Also contemplated are liposomally-encapsulated expression vectors.

Those of skill in the art are well aware of how to apply gene delivery to in vivo and ex vivo situations. For viral vectors, one generally will prepare a viral vector stock. Depending on the kind of virus and the titer attainable, one will deliver 1×10⁴, 1×10⁵, 1×10⁶, 1×10⁷, 1×10⁸, 1×10⁹, 1×10¹⁰, 1×10¹¹ or 1×10¹² infectious particles to the patient. Similar figures may be extrapolated for liposomal or other non-viral formulations by comparing relative uptake efficiencies. Formulation as a pharmaceutically acceptable composition is discussed below.

Various routes are contemplated for various tumor types. The section below on routes contains an extensive list of possible routes. For practically any tumor, systemic delivery is contemplated. This will prove especially important for attacking microscopic or metastatic cancer. Where discrete tumor mass may be identified, a variety of direct, local and regional approaches may be taken. For example, the tumor may be directly injected with the expression vector. A tumor bed may be treated prior to, during or after resection. Following resection, one generally will deliver the vector by a catheter left in place following surgery. One may utilize the tumor vasculature to introduce the vector into the tumor by injecting a supporting vein or artery. A more distal blood supply route also may be utilized.

In a different embodiment, ex vivo gene therapy is contemplated. This approach is particularly suited, although not limited, to treatment of bone marrow associated cancers. In an ex vivo embodiment, cells from the patient are removed and maintained outside the body for at least some period of time. During this period, a therapy is delivered, after which the cells are reintroduced into the patient; hopefully, any tumor cells in the sample have been killed.

Autologous bone marrow transplant (ABMT) is an example of ex vivo gene therapy. Basically, the notion behind ABMT is that the patient will serve as his or her own bone marrow donor. Thus, a normally lethal dose of irradiation or chemotherapeutic may be delivered to the patient to kill tumor cells, and the bone marrow repopulated with the patients own cells that have been maintained (and perhaps expanded) ex vivo. Because, bone marrow often is contaminated with tumor cells, it is desirable to purge the bone marrow of these cells. Use of gene therapy to accomplish this goal is yet another way t-DARPP may be utilized according to the present invention.

B. Protein Therapy

Another therapy approach is the provision, to a subject, of t-DARPP dominant-negative polypeptide, inactive/competitive fragments, synthetic peptides, mimetics or other analogs thereof, or antibodies that bind thereto. The protein/peptide may be produced by recombinant expression means or, if small enough, generated by an automated peptide synthesizer. Formulations would be selected based on the route of administration and purpose including, but not limited to, liposomal formulations and classic pharmaceutical preparations.

C. Combined Therapy with HER-2/Neu Therapy

In a particular embodiment, the present invention contemplates the use of t-DARPP therapies to overcome tumor cell resistance to cancer therapies such as a HER-21Neu therapy like trastuzumab. Trastuzumab (more commonly known under the trade name Herceptin™) is a humanized monoclonal antibody that acts on the extracellular portion of the HER2/Neu (ERBB2) receptor. Trastuzumab's principal use is as an anti-cancer therapy in breast cancer in patients whose tumors over express (produce more than the usual amount of) this receptor. Trastuzumab is administered either once a week or once every three weeks intravenously for 30 to 90 minutes. Amplification of ERBB2 occurs in 25-30% of early-stage breast cancers. It encodes the transmembrane tyrosine kinase p185-erbB2 glycoprotein. Overexpression of HER2/Neu can confer therapeutic resistance to cancer therapies.

Cells treated with trastuzumab undergo arrest during the G1 phase of the cell cycle so there is reduced proliferation. It has been suggested that trastuzumab induces some of its effect by downregulation of ERB2 leading to disruption of receptor dimerization and signaling through the downstream PI3K cascade. P27Kip1 is then not phosphorylated and is able to enter the nucleus and inhibit CDK2 activity, causing cell cycle arrest. Also, trastuzumab suppresses angiogenesis by both induction of anti-angiogenic factors and repression of proangiogenic factors. It is thought that a contribution to the unregulated growth observed in cancer could be due to proteolytic cleavage of ERBB2 that results in the release of the extracellular domain. Trastuzumab has been shown to inhibit ERBB2 ectodomain cleavage in breast cancer cells. There may be other undiscovered mechanisms by which trastuzumab induces regression in cancer.

Initiation of trastuzumab therapy is based upon the identification of HER-2 overexpression. Various methodologies have been developed to identify overexpression of HER-2. In the routine clinical laboratory, the most commonly employed methods are immunohistochemistry (IHC) and either chromogenic or fluorescent in situ hybridization (CISH/FISH). In addition numerous PCR-based methodologies have also been described.

The optimal duration of adjuvant trastuzumab is currently unknown. One year of treatment is generally accepted as the ideal length of therapy based on current clinical trial evidence that demonstrated the superiority of one year treatment over none. However, a small Finnish trial also showed similar improvement with nine weeks' of treatment over no therapy. Due to the lack of direct head to head comparison in clinical trials, it is unknown whether a shorter duration of treatment may just be as effective (with less side effects) than the current accepted practice of treatment for one year. Debate about treatment duration has become a relevant issue for many public health policy makers due to the high financial costs involved in the administration of this treatment for one year. Some countries with a free public health system such as New Zealand, has opted to only fund for nine weeks of adjuvant therapy as a result.^{[14]} Current clinical trials are in progress hoping to answer this question by directly comparing short versus long duration of therapy.

One of the significant complications of trastuzumab is its effect on the heart. Trastuzumab is associated with cardiac dysfunction in 2-7% of cases. Approximately 10% of patients are unable to tolerate this drug because of pre-existing heart problems; physicians are balancing the risk of recurrent cancer against the higher risk of death due to cardiac disease in this population. The risk of cardiomyopathy is increased when trastuzumab is combined with anthracycline chemotherapy (which itself is associated with cardiac toxicity). The present invention thus contemplates the use of lower doses of trastuzumab when used in combination with a t-DARPP inhibitor, thereby avoiding this toxicity. In addition, trastuzumab costs about seventy thousand U.S. dollars for a full course of treatment. The ability to reduce trastuzumab doses can also reduce cost.

D. Other Combinations

In the context of the present invention, it also is contemplated that anti t-DARPP therapy could be used similarly in conjunction with chemo- or radiotherapeutic intervention. It also may prove effective to combine anti-t-DARPP therapy with Her-2/Neu therapy and another therapy, such as chemo- or radiotherapy. Another possible combination involves ADEPT, or Antibody-Directed Enzyme Pro-drug Therapy, a type of treatment that uses monoclonal antibodies to target enzymes to a cancer site. More specifically, the monoclonal antibody carries an enzyme to the cancer cells, and a few hours later, a pro-drug is given. The pro-drug is an inactive anti-cancer drug, but when the pro-drug comes into contact with the enzyme, a reaction takes place which activates the anti-cancer drug. The anti-cancer drug is then able to destroy the cancer cells. As the antibody does not attach to normal cells, the treatment does not affect them.

To kill cells, inhibit cell growth, inhibit metastasis, inhibit angiogenesis or otherwise reverse or reduce the malignant phenotype of tumor cells, using the methods and compositions of the present invention, one would generally contact a “target” cell with a t-DARPP therapy and at least one other agent. These compositions would be provided in a combined amount effective to kill or inhibit proliferation of the cell. This process may involve contacting the cells with the t-Darpp agent and the other agent(s) or factor(s) at the same time. This may be achieved by contacting the cell with a single composition or pharmacological formulation that includes both agents, or by contacting the cell with two distinct compositions or formulations, at the same time, wherein one composition includes the anti-t-DARPP agent and the other includes the other agent.

Alternatively, the anti-t-DARPP therapy treatment may precede or follow the other agent treatment by intervals ranging from minutes to weeks. In embodiments where the other agent and the anti-t-DARPP are applied separately to the cell, one would generally ensure that a significant period of time did not expire between the time of each delivery, such that the agent and expression construct would still be able to exert an advantageously combined effect on the cell. In such instances, it is contemplated that one would contact the cell with both modalities within about 12-24 hours of each other and, more preferably, within about 6-12 hours of each other, with a delay time of only about 12 hours being most preferred. In some situations, it may be desirable to extend the time period for treatment significantly, however, where several days (2, 3, 4, 5, 6 or 7) to several weeks (1, 2, 3, 4, 5, 6, 7 or 8) lapse between the respective administrations.

It also is conceivable that more than one administration of either the anti-t-DARPP or the other agent will be desired. Various combinations may be employed, where the anti-t-DARPP therapy (alone or with a Her-2/Neu therapy) is “A” and the other agent is “B”, as exemplified below:

A/B/A B/A/B B/B/A A/A/B B/A/A A/B/B B/B/B/A
B/B/A/B A/A/B/B A/B/A/B A/B/B/A B/B/A/A B/A/B/A
B/A/A/B B/B/B/A A/A/A/B B/A/A/A A/B/A/A A/A/B/A
A/B/B/B B/A/B/B B/B/A/B

Other combinations are contemplated. Again, to achieve cell killing, both agents are delivered to a cell in a combined amount effective to kill the cell.

Agents or factors suitable for use in a combined therapy are any chemical compound or treatment method that induces DNA damage when applied to a cell. Such agents and factors include radiation and waves that induce DNA damage such as, γ-irradiation, X-rays, UV-irradiation, microwaves, electronic emissions, and the like. A variety of chemical compounds, also described as “chemotherapeutic agents,” function to induce DNA damage, all of which are intended to be of use in the combined treatment methods disclosed herein. Chemotherapeutic agents contemplated to be of use, include, e.g., adriamycin, 5-fluorouracil (5FU), etoposide (VP-16), camptothecin, actinomycin-D, mitomycin C, cisplatin (CDDP) and even hydrogen peroxide. The invention also encompasses the use of a combination of one or more DNA damaging agents, whether radiation-based or actual compounds, such as the use of X-rays with cisplatin or the use of cisplatin with etoposide. In certain embodiments, the use of cisplatin in combination with a T-Darpp expression construct is particularly preferred as this compound.

In treating cancer according to the invention, one would contact the tumor cells with an agent in addition to the expression construct. This may be achieved by irradiating the localized tumor site with radiation such as X-rays, UV-light, γ-rays or even microwaves. Alternatively, the tumor cells may be contacted with the agent by administering to the subject a therapeutically effective amount of a pharmaceutical composition comprising a compound such as, adriamycin, 5-fluorouracil, etoposide, camptothecin, actinomycin-D, mitomycin C, or more preferably, cisplatin. The agent may be prepared and used as a combined therapeutic composition, or kit, by combining it with an anti-t-DARPP therapy, as described above.

Agents that directly cross-link nucleic acids, specifically DNA, are envisaged to facilitate DNA damage leading to a synergistic, antineoplastic combination with an anti-t-DARPP therapy. Agents such as cisplatin, and other DNA alkylating agents may be used. Cisplatin has been widely used to treat cancer, with efficacious doses used in clinical applications of 20 mg/m² for 5 days every three weeks for a total of three courses. Cisplatin is not absorbed orally and must therefore be delivered via injection intravenously, subcutaneously, intratumorally or intraperitoneally.

Agents that damage DNA also include compounds that interfere with DNA replication, mitosis and chromosomal segregation. Such chemotherapeutic compounds include adriamycin, also known as doxorubicin, etoposide, verapamil, podophyllotoxin, and the like. Widely used in a clinical setting for the treatment of neoplasms, these compounds are administered through bolus injections intravenously at doses ranging from 25-75 mg/m² at 21 day intervals for adriamycin, to 35-50 mg/m² for etoposide intravenously or double the intravenous dose orally.

Agents that disrupt the synthesis and fidelity of nucleic acid precursors and subunits also lead to DNA damage. As such a number of nucleic acid precursors have been developed. Particularly useful are agents that have undergone extensive testing and are readily available. As such, agents such as 5-fluorouracil (5-FU), are preferentially used by neoplastic tissue, making this agent particularly useful for targeting to neoplastic cells. Although quite toxic, 5-FU, is applicable in a wide range of carriers, including topical, however intravenous administration with doses ranging from 3 to 15 mg/kg/day being commonly used.

Other factors that cause DNA damage and have been used extensively include what are commonly known as γrays, X-rays, and/or the directed delivery of radioisotopes to tumor cells. Other forms of DNA damaging factors are also contemplated such as microwaves and UV-irradiation. It is most likely that all of these factors affect a broad range of damage DNA, on the precursors of DNA, the replication and repair of DNA, and the assembly and maintenance of chromosomes. Dosage ranges for X-rays range from daily doses of 50 to 200 roentgens for prolonged periods of time (3 to 4 weeks), to single doses of 2000 to 6000 roentgens. Dosage ranges for radioisotopes vary widely, and depend on the half-life of the isotope, the strength and type of radiation emitted, and the uptake by the neoplastic cells.

The skilled artisan is directed to “Remington's Pharmaceutical Sciences” 15th Edition, chapter 33, in particular pages 624-652. Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

The inventors propose that the local or regional delivery of a t-DARPP inhibitor to patients with cancer will be a very efficient method for treating the clinical disease. Similarly, the chemo- or radiotherapy may be directed to a particular, affected region of the subjects body. Alternatively, systemic delivery of expression construct and/or the agent may be appropriate in certain circumstances, for example, where extensive metastasis has occurred.

In addition to combining an anti-t-DARPP therapy with chemo- and radiotherapies, it also is contemplated that combination with other gene therapies will be advantageous. For example, targeting of t-DARPP and p53 mutations at the same time may produce an improved anti-cancer treatment. Any other tumor-related gene conceivably can be targeted in this manner, for example, p21, Rb, APC, DCC, NF-1, NF-2, BCRA2, p16, FHIT, WT-1, MEN-I, MEN-II, BRCA1, VHL, FCC, MCC, ras, myc, neu, raf erb, src, fms, jun, trk, ret, gsp, hst, bcl and abl.

E. Formulations and Routes for Administration to Patients

Where clinical applications are contemplated, it will be necessary to prepare pharmaceutical compositions—expression vectors, virus stocks, proteins, peptides, antibodies and drugs—in a form appropriate for the intended application. Generally, this will entail preparing compositions that are essentially free of pyrogens, as well as other impurities that could be harmful to humans or animals.

One will generally desire to employ appropriate salts and buffers to render delivery vectors stable and allow for uptake by target cells. Buffers also will be employed when recombinant cells are introduced into a patient. Aqueous compositions of the present invention comprise an effective amount of the vector to cells, dissolved or dispersed in a pharmaceutically acceptable carrier or aqueous medium. Such compositions also are referred to as inocula. The phrase “pharmaceutically or pharmacologically acceptable” refer to molecular entities and compositions that do not produce adverse, allergic, or other untoward reactions when administered to an animal or a human. As used herein, “pharmaceutically acceptable carrier” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the vectors or cells of the present invention, its use in therapeutic compositions is contemplated. Supplementary active ingredients also can be incorporated into the compositions.

The active compositions of the present invention may include classic pharmaceutical preparations. Administration of these compositions according to the present invention will be via any common route so long as the target tissue is available via that route. This includes oral, nasal, buccal, rectal, vaginal or topical. Alternatively, administration may be by orthotopic, intradermal, subcutaneous, intramuscular, intraperitoneal or intravenous injection. Such compositions would normally be administered as pharmaceutically acceptable compositions, described supra. Of particular interest is direct intratumoral administration, perfusion of a tumor, or administration local or regional to a tumor, for example, in the local or regional vasculature or lymphatic system, or in a resected tumor bed.

The active compounds may also be administered parenterally or intraperitoneally. Solutions of the active compounds as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions. In all cases the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with several of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

As used herein, “pharmaceutically acceptable carrier” includes any and all solvents, dispersion media, coatings, antibacterial and antifungal agents, isotonic and absorption delaying agents and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions.

For oral administration the polypeptides of the present invention may be incorporated with excipients and used in the form of non-ingestible mouthwashes and dentifrices. A mouthwash may be prepared incorporating the active ingredient in the required amount in an appropriate solvent, such as a sodium borate solution (Dobell's Solution). Alternatively, the active ingredient may be incorporated into an antiseptic wash containing sodium borate, glycerin and potassium bicarbonate. The active ingredient may also be dispersed in dentifrices, including: gels, pastes, powders and slurries. The active ingredient may be added in a therapeutically effective amount to a paste dentifrice that may include water, binders, abrasives, flavoring agents, foaming agents, and humectants.

The compositions of the present invention may be formulated in a neutral or salt form. Pharmaceutically-acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

Upon formulation, solutions will be administered in a manner compatible with the dosage formulation and in such amount as is therapeutically effective. The formulations are easily administered in a variety of dosage forms such as injectable solutions, drug release capsules and the like. For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media which can be employed will be known to those of skill in the art in light of the present disclosure. For example, one dosage could be dissolved in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

### VI. Kits

According to the present invention, there are provided kits for detecting t-DARPP expression. The kit of the present invention can be prepared by known materials and techniques which are conventionally used in the art. Generally, kits comprise separate vials or containers for the various reagents, such as probes, primers, enzymes, antibodies, etc. The reagents are also generally prepared in a form suitable for preservation by dissolving it in a suitable solvent. Examples of a suitable solvent include water, ethanol, various buffer solutions, and the like. The various vials or containers are often held in blow-molded or injection-molded plastics.

### VII. Examples

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Materials & Methods

Cell lines and trastuzumab: BT-474 and SKBR-3 were obtained from the American Type Tissue Culture Collection (Manassas, Va.). To obtain Herceptin resistant cell lines, BT-474 cells were xenografted in athymic nude mice and were eliminated by trastuzumab as described previously (Ritter et al., 2007). Briefly, continuous cell lines (HR for Herceptin resistant) were generated from tumors that recurred in the presence of antibody therapy. Twenty mg/kg trastuzumab were diluted in sterile PBS and given by i.p. injection twice a week. The isolated cells behaved resistant to trastuzumab in culture as well as when re-injected into nude mice (For details, (Ritter et al., 2007)). All cells were maintained in Improved Minimal Essential Medium (IMEM; Life Technologies, Inc., Rockville, Md.) containing 10% FCS (Hyclone, Logan, Utah) at 37° C. in a humidified, 5% CO₂ atmosphere. Herceptin was purchased from the Vanderbilt University Hospital Pharmacy (Nashville, Tenn.).

Vectors: The expression plasmid for t-DARPP was generated by PCR amplification of the full-length coding sequence of t-DARPP and cloned in frame into pcDNA3.1 (Invitrogen Life Technologies, Carlsbad, Calif.). Cloning of t-DARPP was confirmed by sequencing and restriction enzyme digestion as described elsewhere (Belkhiri et al., 2005). Stably transfected SKBR-3 cells expressing t-DARPP or pcDNA3.1 empty vector were generated as described previously (Belkhiri et al., 2005). After selection with 400 μg/ml of neomycin (Invitrogen Life Technologies, Carlsbad, Calif.), the resulting resistant clones were screened for t-DARPP protein expression by Western blot analysis.

Cell survival assay: 5×10³ cells (BT474, HR5 and HR6 cells) per well were plated in a 96-well plate. The survival of these cells after treatment with vehicle or trastuzumab, or knockdown of t-Darpp was assayed using the CellTiter-Glo Luminescent Cell Viability Assay Kit (Promega, Madison, Wis.) following the supplier's instructions. This assay has high sensitivity in measurement of cell survival and can detect as little as a single cell or 0.01 picomoles of ATP. Cell washing, medium removal, and multiple pipeting are not required, thus providing higher reproducibility.

Immunoblot analysis: Sub-confluent cell monolayers were harvested by scraping in ice-cold PBS and spinning down at 3500 rpm in a bench top centrifuge for 10 minutes. Cell pellets were then lysed in RIPA buffer containing 1% Halt protease inhibitor cocktail (Pierce Biotechnology, Rockford, Ill.). Protein concentration was determined by standard Bradford assay, and total cell lysates (10 μg/lane) were separated by 10% SDS-PAGE and subjected to immunoblot analysis. Gel loading was normalized for equal β-actin. Proteins were then transferred onto Hybond-P PVDF membranes (Amersham Biosciences, Little Chalfont, UK). Horseradish peroxidase-conjugated secondary antibodies were obtained from Amersham Biosciences (Little Chalfont, UK). Immunoreactive bands were visualized by enhanced chemiluminescence (ECL; Pierce, Rockford, Ill.). A carboxyl terminal antibody that recognizes t-DARPP was obtained from Santa Cruz Biotechnology (Santa Cruz, Calif.). ERBB2, AKT, p-Ser₄₇₃ AKT, cleaved caspase 3, HSP90, and P-actin antibodies were obtained from Cell Signaling (Beverly, Mass.).

Quantitative real-time PCR: Quantitative real-time PCR (qPCR) was performed using an iCycler (Bio-Rad, Hercules, Calif.) with a threshold cycle number determined by use of iCycler software, version 3.0. Reactions were performed in triplicate and threshold cycle numbers were averaged. For qRT-PCR, single-stranded cDNA was synthesized using the Advantage RT-PCR Kit (Clontech, Palo Alto, Calif.). Gene-specific primers for DNA and mRNA specific sequences of t-DARPP, ERBB2, β-actin and HPRT1 were designed, and the results were normalized to β-actin and HPRT1, which are considered reliable and stable reference genes for real-time PCR (Schwartz et al., 2003). The mRNA expression-fold was calculated according to the formula 2_{(Rt-Et)}/2_{(Rn-En)}, as described previously (E1-Rifai et al., 2002), where Rt is the threshold cycle number for the reference gene observed in the test sample; Et is the threshold cycle number for the experimental gene observed in the test sample; Rn is the threshold cycle number for the reference gene observed in the normal sample; and En is the threshold cycle number for the experimental gene observed in the normal sample. Rn and En values were the averages taken from all normal analyzed samples.

Gene expression knockdown by small-interfering RNA (siRNA): The BT-474, HR5 and HR6 cells were transfected with control siRNA (sc-37007) or t-DARPP siRNA (sc-35173, a pool of 3 t-DARPP-specific siRNA oligonucleotides) using siRNA transfection reagent (sc-29528), and transfection medium (sc-36868) following the manufacturer's instructions (Santa Cruz Biotechnology, Santa Cruz, Calif.). Cell lysates were subjected to Western blot analysis of ERBB2, t-DARPP, cleaved caspase 3. In addition, AKT, p-Ser₄₇₃ AKT, HSP90, and BCL2 were analyzed in HR6 cells. All antibodies were obtained from Cell Signaling (Cell Signaling Technology, Inc, Danvers, Mass.). Gel loading was normalized for equal β-actin (10 μg protein per lane).

DNA bisulfite treatment and Pyrosequencing analysis of DNA methylation: A CpG island was found in t-DARPP promoter from −1438 to −830 of transcription start site. To investigate if DNA methylation of this CpG island is responsible for the difference of expression of t-DARPP in these cell lines, the inventors designed a Pyrosequencing assay using PSQ assay design software (Biotage, Kungsgatan 76, Sweden) which quantitatively detects DNA methylation level of 10 CpG sites within −1161 to −1109 of t-DARPP transcription start site. A sequencing primer was designed and used to read the 10 CpG sites. All primers were purchased from Integrated DNA Technologies, Inc. DNA from three cell lines (BT-474, HR5, HR6) was purified using DNeasy kit (Qiagen, Germany). One pg of DNA was modified by bisulfite using EZ DNA Methylation-Gold Kit (ZYMO RESEARCH) according to manufacturer's protocol. Twenty ng of bisulfite-modified DNA was subjected to PCR amplification. The Platinum PCR SuperMix High fidelity (Invitrogen) which contains anti-Taq DNA polymerase antibody, providing an automatic “hot start” was used in PCR reaction amplification. The PCR products were checked by gel-electrophoresis to confirm the products of specific size and no formation of primer dimer. The specific PCR products were then subjected to pyrosequencing quantitative analysis by using Biotage PyroMark MD System (Biotage) according the protocol provided by the manufacturer using a sequencing. The results were analysed by Pyro Q-CPG 1.0.9 software (Biotage).

### Example 2

### Results

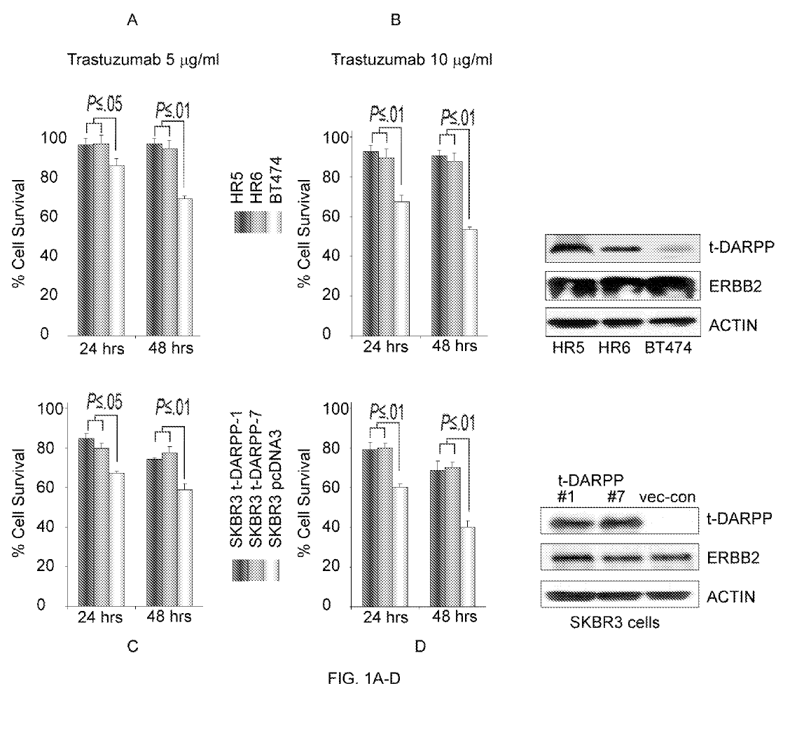

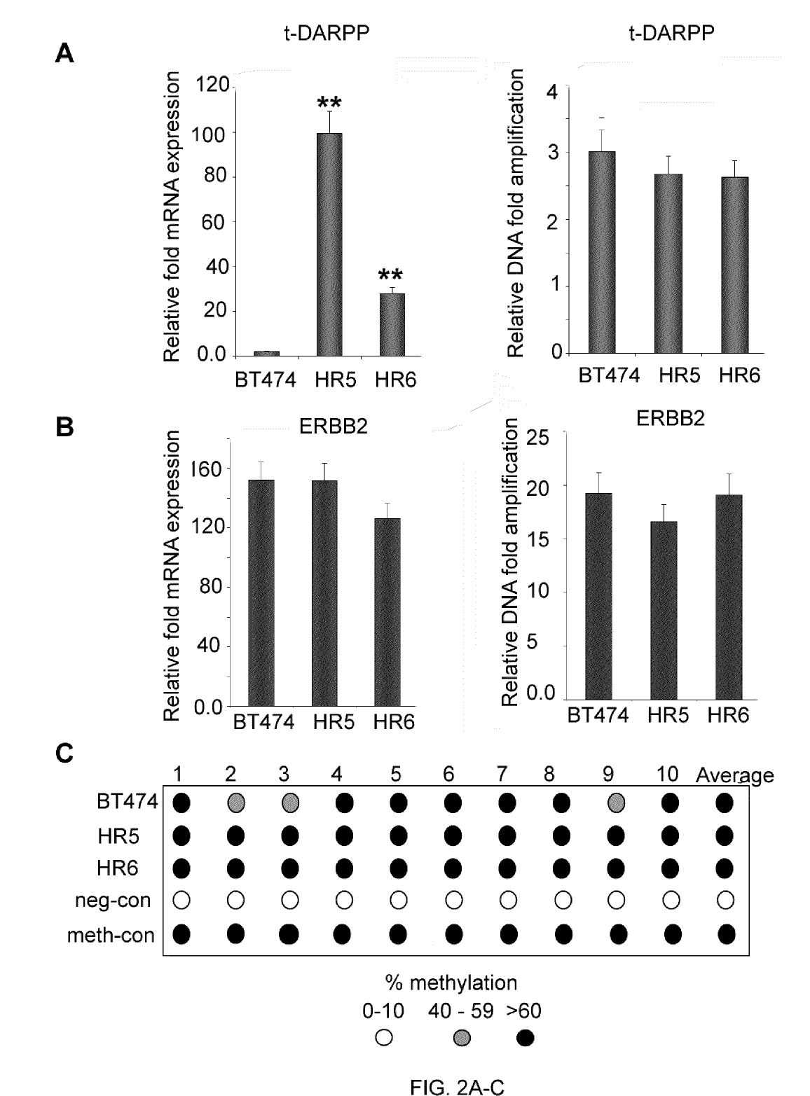

Expression of t-DARPP and trastuzumab resistance. Trastuzumab-sensitive BT-474 and SKBR-3 human breast cancer cells are known to display DNA amplification, mRNA and protein overexpression of ERBB2 (Alimandi et al., 1995; Schwartze, 1987). Trastuzumab-resistant HR5 and HR6 cell lines were derived from BT-474 cells that were xenografted in athymic nude mice and periodically exposed to trastuzumab as described previously (Ritter et al., 2007). Resistance to trastuzumab by HR cells was confirmed by increased cell survival following treatment with trastuzumab (5 and 10 μg/ml) for 24 h and 48 h (FIG. 1A). As the inventors have reported previously that DARPP-32 and its truncated form t-DARPP were overexpressed in the majority gastrointestinal carcinomas and provided resistance against apoptosis (Belkhiri et al., 2005), the inventors determined their expression levels by Western blot analysis in BT-474 and HR cells. Interestingly, t-DARPP protein levels in HR cells were considerably higher as compared to the parental BT-474 cells (FIG. 1B). In contrast, ERBB2 protein levels remained unchanged in all cells (FIG. 1B). Unlike t-DARPP, DARPP-32 protein was not detected in BT-474, HR, and SKBR-3 cells (data not shown). These results suggest that t-DARPP overexpression correlate with resistance to trastuzumab and increased cell survival without affecting ERBB2 protein levels. To further confirm this finding, the inventors generated SKBR-3 stable cell line expressing t-DARPP, and similar to HR cells, two clones of SKBR-3-t-DARPP cells were significantly resistant to trastuzumab as compared to the empty vector control SKBR-3 cells (FIG. 1C). Moreover, immunoblotting indicated that ERBB2 protein levels were comparable in all cells (FIG. 1D). To determine whether t-DARPP was transcriptionally upregulated and associated with gene amplification, the inventors performed qPCR of t-DARPP. The results demonstrated 25- to 100-fold up-regulation of the t-DARPP transcript levels in HR5 and HR6 clones as compared to the BT-474 parental cell line (FIG. 2A). In contrast, t-DARPP gene amplification was similar in all cell lines, showing three-fold amplification as compared to normal controls (FIG. 2A). ERBB2 mRNA overexpression and gene amplification levels were comparable in BT-473, HR5 and HR6 cells (FIG. 2B). These results indicate that the coamplification of t-DARPP and ERBB2 was not driving the expression of t-DARPP where it remained transcriptionally repressed in trastuzumab sensitive cell line (BT-474) but became transcriptionally upregulated in HR5 and HR6 cells. The inventors have attempted to investigate if a methylation-dependent epigenetic mechanism was involved in the transcriptional regulation of t-DARPP in BT-474 and HR cells. The results of pyrosequencing analysis of 10 CpG sites within −1161 to −1109 region of t-DARPP putative promoter indicated comparable high levels of DNA methylation in BT-474, HR5 and HR6 cells (FIG. 2C). This suggested that DNA methylation of the t-DARPP promoter CpG island did not regulate the expression of t-DARPP. Therefore, the molecular mechanism(s) underlying the transcription upregulation of t-DARPP remain unknown in this study.

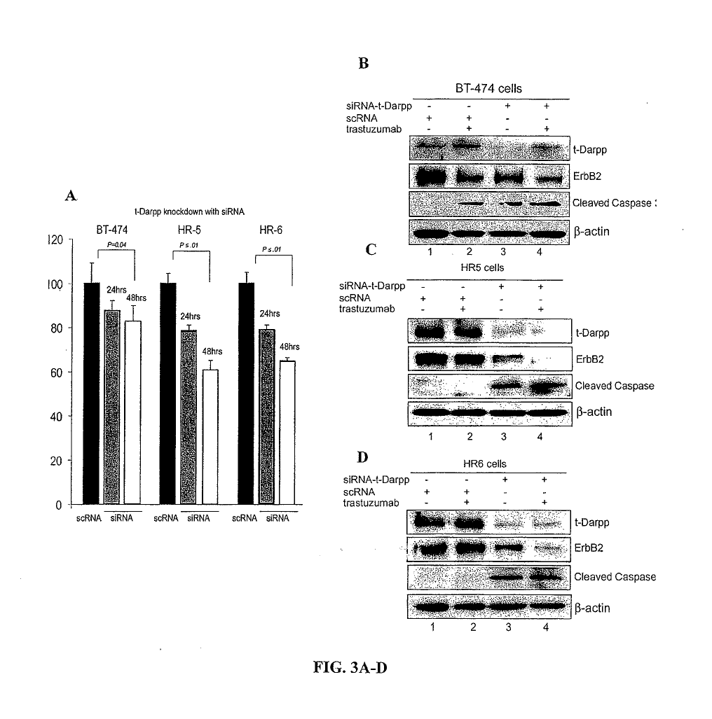

t-DARPP mediates cell survival and its knockdown leads to cell death in HR5 and HR6 trastuzumab resistant cells. The inventors have earlier shown that t-DARPP can mediate cell survival in epithelial cells (Belkhiri et al., 2005). Since t-DARPP was overexpressed in HR5 and HR6 cells, the inventors performed a knockdown of t-DARPP in these cells to ascertain its role in trastuzumab resistance. Indeed, knockdown of t-DARPP led to a significant reduction in cell survival at 24 hr and 48 hr post-transfection as compared to scrambled siRNA control (P ≦0.001) (FIG. 3A). Interestingly, BT-474 showed a reduction in cell survival that was less significant than HR5 and HR6 and correlated with the relative low expression of t-DARPP in these cells underscoring the possibility that t-DARPP levels in the cell may pre-determine the sensitivity to its selective knockdown (FIGS. 3A & 1A). To ascertain that knockdown of t-DARPP induced apoptosis, the inventor knocked down t-DARPP alone and in combination with 20 μg/ml trastuzumab treatment of BT-474, HR5 and HR6 cells. As expected, trastuzumab alone downregulated ERBB2 protein level and induced apoptosis as indicated by appearance of cleaved caspase 3 fragment in BT-474 cells (FIG. 3B, lane 2). Knockdown of t-DARPP alone or in combination with trastuzumab significantly downregulated ERBB2 and activated caspase 3 in BT-474 cells (FIG. 3B, lanes 3 & 4). In contrast, trastuzumab alone did not down-regulate ERBB2 or activate caspase 3 in HR5 (FIG. 3C, lane 2) and HR6 (FIG. 3D, lane 2). On the other hand, knockdown of t-DARPP alone or in combination with trastuzumab downregulated ERBB2 and activated caspase 3 in HR5 (FIG. 3C, lanes 3 & 4) and HR6 (FIG. 3D, lanes 3 & 4). Taken together, these results indicate that knockdown of t-DARPP alone was sufficient to down-regulate ERBB2 and induce apoptosis in HR5 and HR6 trastuzumab resistant cells.

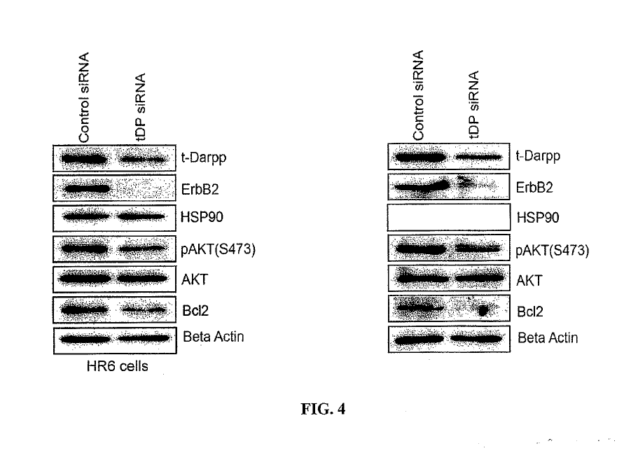

Knockdown of t-DARPP leads to downregulation, of ERBB2 protein and inhibition of the PI3K/AKT survival pathway in trastuzumab-resistant cells. Since the inventors' results described above demonstrated that t-DARPP mediated cell survival and provided resistance to trastuzumab, the inventors investigated the role of t-DARPP in promoting survival through the PI3K/AKT pathway. The inventors knocked down t-DARPP in HR6 cells, as representative of trastuzumab-resistant cells, and Western blot analysis showed a significant downregulation of ERBB2 protein associated with a notable decrease of p-Ser₄₇₃ AKT without affecting total AKT protein level (FIG. 4). Interestingly, the prosurvival protein BCL2 was also downregulated following t-DARPP knockdown leading to induction of apoptosis as indicated by appearance of cleaved caspase 3 (FIG. 4). As the heat shock protein HSP90 was reported to stabilize expression of ERBB2 through binding (Peng et al., 2005), the inventors investigated whether t-DARPP could regulate the expression of HSP90. The inventors' results showed that knockdown of t-DARPP did not affect HSP90 protein level (FIG. 4).

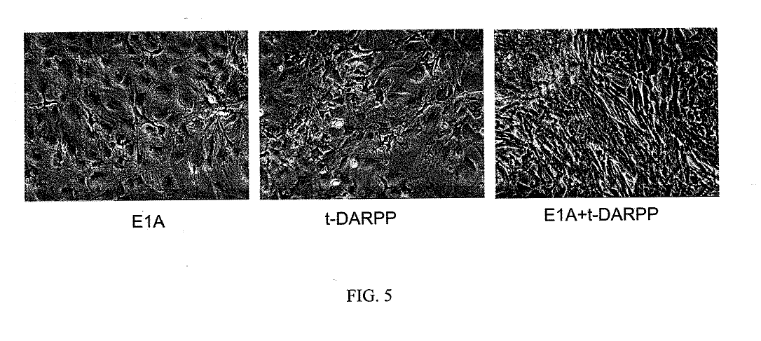

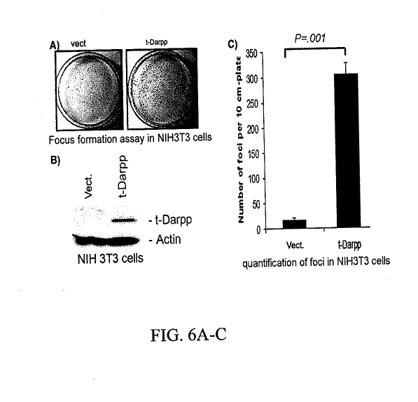

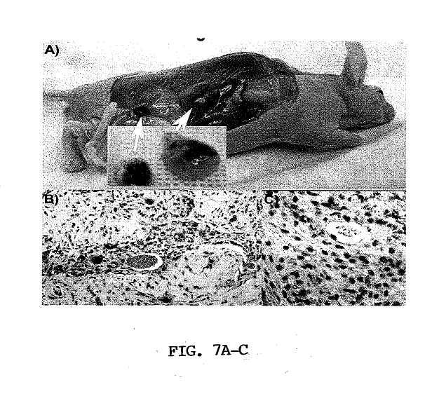

Identification of Oncogenic Potential of t-DARPP. The inventors performed gene transfer experiments that included focus formation assay, a classical transformation assay in primary mouse embryonic fibroblasts, NIH3T3 cells, and xenografting. The results indicated that t-DARPP transforms MEFs and NIH3T3 cells (FIGS. 5 and 6A-C). They then followed up on this finding by xenografting 2×10⁶ NIH3T3 cells transduced with t-DARPP, or empty vector (n=5 for each). The results indicated that t-DARPP led to the formation of tumors in 3/5 mice, whereas no tumors were seen for empty vector after 6 weeks of xenografting (FIGS. 7A-C). These tumors were classified by a pathologist as high grade sarcoma.

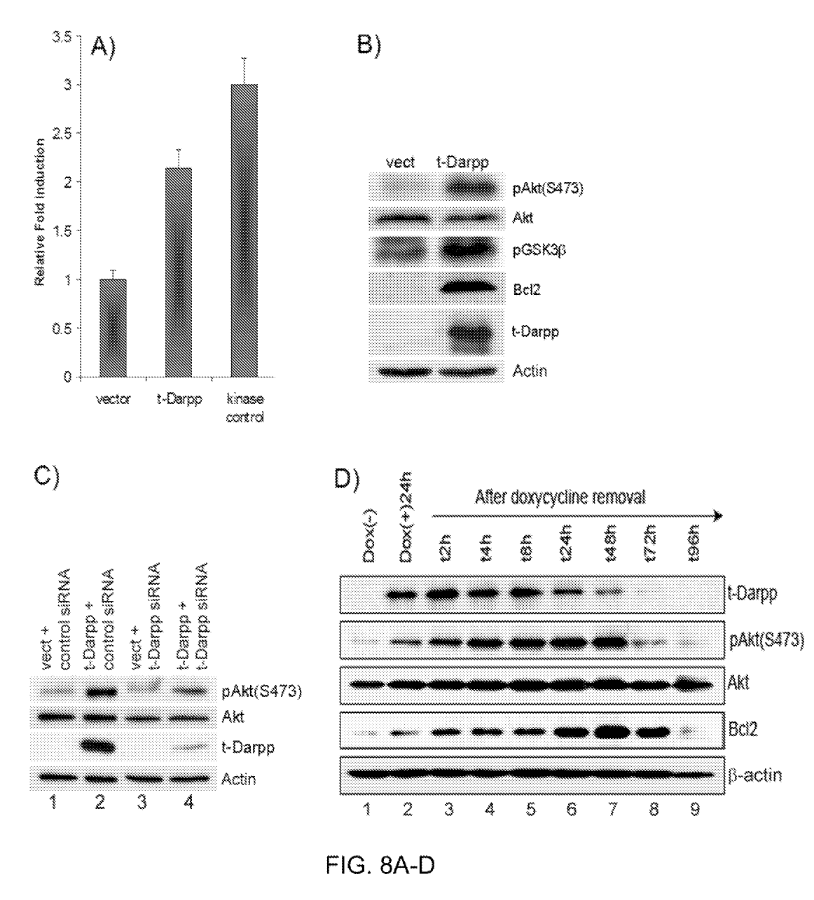

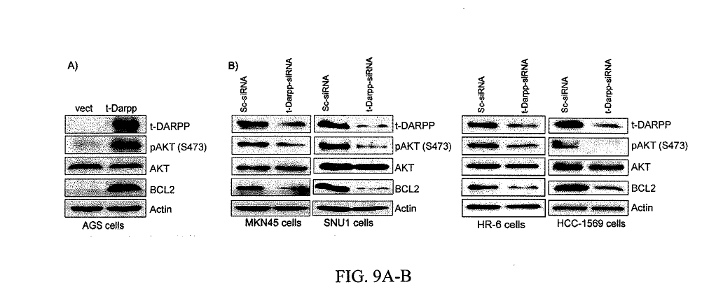

t-DARPP Regulates AKT Activity and BCL-2 Levels. Protein extracts from corresponding time points were analyzed by immunoblotting for p-Ser₄₇₃ AKT, AKT, BCL-2, and t-DARPP. As expected, the results in FIG. 8D show the t-DARPP protein level increased after 24 hr of induction of cells with doxycycline (lane 2), remained high for 8 hr after removal of doxycycline (lanes 3-5), and substantially decreased after 24 hours as shown in lanes 6-8. Similarly, AKT phosphorylation increased after induction with doxycycline and the signal level remained high following the removal of doxycycline for 48 hr (lanes 2-7), but dramatically decreased after 72 hr (lane 8). BCL-2 protein level increased after doxycycline induction and sustained its levels after the removal of doxycycline up to 72 hr as indicated in lanes 2-8. Together, our data confirm that t-DARPP mediates activation of the AKT pathway and up-regulates BCL-2 (Belkhiri et al. 2008a). Further validation was performed by knocking down endogenous t-DARPP in several cancer cell lines (FIGS. 9A-B).

t-DARPP Inhibition Reduces Cancer Cell/Tumor Growth in Mice. A mystery surrounds targeted anticancer drugs like trastuzumab (Herceptin), imatinib (Gleevec), and erlotinib (Tarceva), agents that block the specific signaling pathways. Cancer cells contain multiple genetic and epigenetic abnormalities. Despite this complexity, their growth and survival can often be impaired by the inactivation of a single oncogene (Rothenberg et al., 2008). This phenomenon, called “oncogene addiction,” provides a rationale for molecular targeted therapy (Rothenberg et al., 2008; Felsher, 2008a). Combination therapy may also be required to prevent the escape of cancers from a given state of oncogene addiction (Weinstein and Joe, 2006; 2008). Experimental evidence surprisingly illustrates that the inactivation of even a single oncogene can be sufficient to induce sustained tumor regression. The proposed explanation for this phenomenon is that activated oncogenes result in a signaling state in which the sudden abatement of oncogene activity balances towards proliferative arrest and apoptosis (Felsher, 2008b). Understanding when and how oncogene inactivation induces apoptosis, differentiation, and senescence within a tumor will be important when developing effective strategies for the treatment of cancer (Felsher, 2008a).

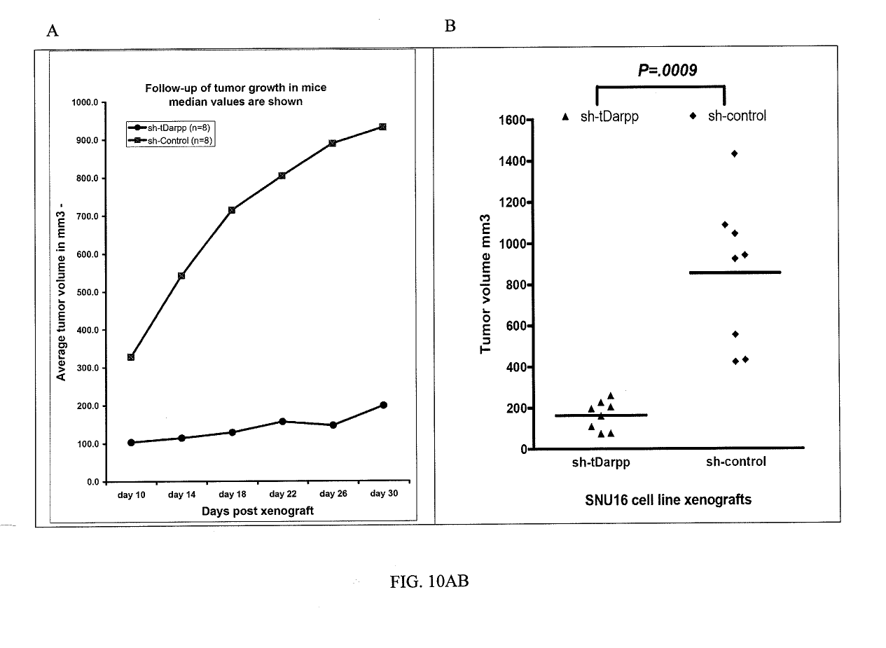

In light of these biological phenomena and the inventors' data that demonstrate that t-Darpp is an oncogene that transforms primary mouse embryonic fibroblasts, the inventors sought to examine the hypothesis of oncogene addiction and whether cancer cells that express t-DARPP are dependent on its expression for their survival. They used xenografted animal model utilizing human cancer cell lines that were engineered to express a tet-inducible shRNA t-DARPP or empty vector. Treatment with doxycycline leads to knockdown of t-DARPP in cells expressing the tet-inducible shRNA t-DARPP vector whereas cells with empty vector will have no change in the level of t-DARPP. The inventors are applying this approach on three human cancer cell lines that have high levels of endogenous t-DARPP (SNU16, MKN45, and HCC1569). For each cell line, the inventors randomized 16 mice and generated 8 xenografts with cell lines expressing the sh-t-DARPP and another eight with empty vector. All mice received doxycycline in drinking water, and the mice are followed for 30 days. For SNU16, at the end of the 30 days, none of the xenografts that express the shRNA t-DARPP had any significant growth and they remained under 250 mm³ (FIGS. 10A-B). On the other hand, the control group had fast growing tumors that reached and exceeded the size of 1000 mm³, requiring immediate termination according to the institutional animal care approved protocols (FIGS. 10A-B). In fact, three out of eight mice in the control group were terminated before 20 days due to the large tumor size (FIGS. 10A-B). These results confirm that t-DARPP is a critical pro-survival protein and that cancer cells that express this protein become dependent on its expression for their survival and growth. Moreover, these data support the conclusion that cancer cells can be addicted to t-DARPP and that knockdown of its expression is an anti-cancer therapy approach.

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. More specifically, it will be apparent that certain agents that are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### VIII. References

The following references, to the extent that they provide exemplary procedural or other details supplementary to those set forth herein, are specifically incorporated herein by reference:
U.S. Pat. No. 4,196,265
U.S. Pat. No. 4,415,723
U.S. Pat. No. 4,458,066
U.S. Pat. No. 4,554,101
U.S. Pat. No. 4,683,195
U.S. Pat. No. 4,683,202
U.S. Pat. No. 4,684,611
U.S. Pat. No. 4,800,159
U.S. Pat. No. 4,879,236
U.S. Pat. No. 4,883,750
U.S. Pat. No. 4,952,500
U.S. Pat. No. 5,217,879
U.S. Pat. No. 5,279,721
U.S. Pat. No. 5,302,523
U.S. Pat. No. 5,322,783
U.S. Pat. No. 5,354,855
U.S. Pat. No. 5,384,253
U.S. Pat. No. 5,440,013
U.S. Pat. No. 5,446,128
U.S. Pat. No. 5,464,765
U.S. Pat. No. 5,475,085
U.S. Pat. No. 5,506,138
U.S. Pat. No. 5,538,877
U.S. Pat. No. 5,538,880
U.S. Pat. No. 5,550,318
U.S. Pat. No. 5,563,055
U.S. Pat. No. 5,580,859
U.S. Pat. No. 5,589,466
U.S. Pat. No. 5,610,042
U.S. Pat. No. 5,618,914
U.S. Pat. No. 5,656,610
U.S. Pat. No. 5,670,155
U.S. Pat. No. 5,670,488
U.S. Pat. No. 5,672,681
U.S. Pat. No. 5,674,976
U.S. Pat. No. 5,702,932
U.S. Pat. No. 5,710,245
U.S. Pat. No. 5,736,524
U.S. Pat. No. 5,739,018
U.S. Pat. No. 5,780,448
U.S. Pat. No. 5,789,215
U.S. Pat. No. 5,795,715
U.S. Pat. No. 5,824,544
U.S. Pat. No. 5,830,725
U.S. Pat. No. 5,840,833
U.S. Pat. No. 5,849,304
U.S. Pat. No. 5,851,826
U.S. Pat. No. 5,858,744
U.S. Pat. No. 5,859,184
U.S. Pat. No. 5,871,982
U.S. Pat. No. 5,871,983
U.S. Pat. No. 5,871,986
U.S. Pat. No. 5,879,934
U.S. Pat. No. 5,888,502
U.S. Pat. No. 5,889,136
U.S. Pat. No. 5,925,565
U.S. Pat. No. 5,928,906
U.S. Pat. No. 5,929,237
U.S. Pat. No. 5,932,210
U.S. Pat. No. 5,935,819
U.S. Pat. No. 5,945,100
U.S. Pat. No. 5,955,331
U.S. Pat. No. 5,981,274
U.S. Pat. No. 5,994,136
U.S. Pat. No. 5,994,624
U.S. Pat. No. 6,013,516
U.S. Pubn 2005/0203047
EPO 320 308
EPO 027 3085
EPO 329 822
GB Appn. 2 202 328
PCT Appln. PCT/US87/00880
PCT Appln. PCT/US89/01025
PCT Appln. WO 88/10315
PCT Appln. WO 89/06700
PCT Appln. WO 90/07641
PCT Appln. WO 00/44914
PCT Appln. WO 01/36646
PCT Appln. WO 01/68836
PCT Appln. WO 89/06700
PCT Appln. WO 90/07641
PCT Appln. WO 9217598
PCT Appln. WO 94/09699
PCT Appln. WO 95/06128
PCT Appln. WO 99/32619
Alimandi et al., Oncogene, 10:1813-1821, 1995.
Almendro et al., J. Immunol., 157(12):5411-5421, 1996.
Amado and Chen, Science, 285(5428):674-676, 1999.
Angel et al., Cell, 49:729, 1987b.
Angel et al., Mol. Cell. Biol., 7:2256, 1987a.
Armentano et al., Proc. Natl. Acad. Sci. USA, 87(16):6141-6145, 1990.
Arteaga et al., Semin. Oncol., 29:4-10, 2002.
Atchison and Perry, Cell, 46:253, 1986.
Atchison and Perry, Cell, 48:121, 1987.
Ausubel et al., In: Current Protocols in Molecular Biology, John, Wiley & Sons, Inc, New York, 1994.
Banerji et al., Cell, 27 (2 Pt 1):299-308, 1981.
Banerji et al., Cell, 33(3):729-740, 1983.
Barany and Merrifield, In: The Peptides, Gross and Meienhofer (Eds.), Academic Press, NY, 1-284, 1979.
Baselga and Albanell, Ann. Oncol., 12 Suppl 1:S35-41, 2001.
Baselga et al., Cancer Res., 58:2825-2831, 1998.
Bates, Mol. Biotechnol., 2(2):135-145, 1994.
Batra et al., Am. J. Respir. Cell Mol. Biol., 21(2):238-245, 1999.
Battraw and Hall, Theor. App. Genet., 82(2):161-168, 1991.
Beckler et al., Cancer, 98:1547-1551, 2003.
Belkhiri et al., Cancer Res., 68(2):395-403, 2008a.
Belkhiri et al., Clin Cancer Res., 14(14):4564-71, 2008b.
Belkhiri et al., Cancer Res., 65:6583-6592, 2005.
Bellus, J. Macromol. Sci. Pure Appl. Chem., A31(1): 1355-1376, 1994.
Berkhout et al., Cell, 59:273-282, 1989.
Bernstein et al., Nature, 409:363-366, 2001.
Bett et al., J Virololgy, 67(10):5911-5921, 1993.
Bhattacharjee et al., J Plant Bioch. Biotech., 6(2):69-73. 1997.
Bilbao et al., Transplant Proc., 31(1-2):792-793, 1999.
Blackwell et al., Arch. Otolaryngol. Head. Neck Surg., 125(8):856-863, 1999.
Blanar et al, EMBO J, 8:1139, 1989.
Blomer et al., J. Virol., 71(9):6641-6649, 1997.
Bodine and Ley, EMBO J, 6:2997, 1987.
Borg et al., Lancet., 1:1268-1269, 1989.
Boshart et al., Cell, 41:521, 1985.
Bosher and Labouesse, Nat. Cell. Biol., 2(2):E31-E36, 2000.
Bosze et al., EMBO J, 5(7):1615-1623, 1986.
Braddock et al., Cell, 58:269, 1989.
Brennecke et al., Cell, 113(1):25-36, 2003.
Brinster et al., Proc. Natl. Acad. Sci. USA, 82(13):4438-4442, 1985.
Bulla and Siddiqui, J. Virol., 62:1437, 1986.
Campbell and Villarreal, Mol. Cell. Biol., 8:1993, 1988.
Campbell et al., Am. Rev. Respir. Dis., 130(3):417-423, 1984.
Campere and Tilghman, Genes and Dev., 3:537, 1989.
Campo et al., Nature, 303:77, 1983.
Capaldi et al., Biochem. Biophys. Res. Comm., 76:425, 1977.
Caplen et al., Gene Ther., 6(3):454-459, 1999.
Caplen et al., Gene, 252(1-2):95-105, 2000.
Carbonelli et al., “FEMS Microbiol Lett. 177(1):75-82, 1999.
Case et al., Proc. Natl. Acad. Sci. USA, 96(6):2988-2893, 1999.
Celander and Haseltine, J Virology, 61:269, 1987.
Celander et al., J Virology, 62:1314, 1988.
Chan et al., Breast Cancer Res. Treat., 91:187-201, 2005.
Chandler et al., Cell, 33:489, 1983.
Chandler et al., Proc. Natl. Acad. Sci. USA, 94(8):3596-601, 1997.
Chang et al., Mol. Cell. Biol., 9:2153, 1989.
Chatterjee et al., Proc. Natl. Acad. Sci. USA, 86:9114, 1989.
Chen and Okayama, Mol. Cell. Biol. 7:2745-2752, 1987.
Chen et al., Genes Dev., 10:2438-2451, 1996.
Chillon et al., J. Virol., 73(3):2537-2540, 1999.
Cho et al., Biotechniques, 30:562-572, 2001.
Choi et al., Cell, 53:519, 1988.
Christou et al., Proc. Natl. Acad. Sci. USA, 84(12):3962-3966, 1987.
Clay et al., Pathol. Oncol. Res., 5(1):3-15,1999.
Cocea, Biotechniques, 23(5):814-816, 1997.
Coffey et al., Science, 282(5392):1332-1334, 1999.
Cohen et al., J. Cell. Physiol., 5:75, 1987.
Cook et al., Cell, 27:487-496, 1981.
Cory and Adams, Nat. Rev. Cancer, 2:647-656, 2002.
Costa et al., Mol. Cell. Biol., 8:81, 1988.
Cripe et al., EMBO J, 6:3745, 1987.
Culotta and Hamer, Mol. Cell. Biol., 9:1376, 1989.
Culver et al., Science, 256(5063):1550-1552, 1992.
Dandachi et al., Anticancer Res., 24:2401-2406, 2004.
Dandolo et al., J Virology, 47:55-64, 1983.
De Villiers et al., Nature, 312(5991):242-246, 1984.
DeLuca et al., J. Virol., 56(2):558-570, 1985.
Derby et al., Hear Res., 134(1-2):1-8,1999.
Deschamps et al., Science, 230:1174-1177, 1985.
D'Halluin et al., Plant Cell, 4(12):1495-1505, 1992.
Doench et al., Genes Dev., 17:438-42, 2003.
Dorai et al., Int. J. Cancer, 82(6):846-852, 1999.
Ebihara et al., Br. J. Cancer, 91:119-123, 2004.
Edbrooke et al., Mol. Cell. Biol., 9:1908, 1989.
Edlund et al., Science, 230:912-916, 1985.
El-Rifai et al., Cancer Res., 62:4061-4064, 2002.
El-Rifai et al., Cancer Res., 62:6823-6826, 2002.
Elbashir et al., Genes Dev., 5(2):188-200, 2001.
Engel and Kohn, Front Biosci., 4:e26-33, 1999.
Fechheimer, et al., Proc Natl. Acad. Sci. USA, 84:8463-8467, 1987.
Feldman et al., Semin. Interv. Cardiol., 1(3):203-208,1996.
Felsher, D W, Apmis, 116:629-37, 2008a.
Felsher, D W, Cancer Res. 68:2081-6, 2008b.
Feng and Holland, Nature, 334:6178, 1988.
Feng et al., Nat. Biotechnol., 15(9):866-870, 1997.
Firak and Subramanian, Mol. Cell. Biol., 6:3667, 1986.
Fire et al., Nature, 391(6669):806-811, 1998.
Fisher et al., Virology, 217(1):11-22, 1996.
Fodor et al., Science, 251:767-773, 1991.
Foecking and Hofstetter, Gene, 45(1):101-105, 1986.
Forster and Symons, Cell, 49:211-220, 1987.
Fraley et al., Proc. Natl. Acad. Sci. USA, 76:3348-3352, 1979.
Freifelder, In: Physical Biochemistry Applications to Biochemistry and Molecular Biology, 2nd Ed. Wm. Freeman and Co., NY, 1982.
Frohman, In: PCR Protocols. A Guide To Methods And Applications, Academic Press, N.Y., 1990.
Fujita et al., Cell, 49:357, 1987.
Fujiwara and Tanaka, Nippon Geka Gakkai Zasshi, 99(7):463-468, 1998.
Garoff and Li, Curr. Opin. Biotechnol., 9(5):464-469, 1998.
Garrido et al., J. Neurovirol., 5(3):280-288, 1999.
Gefter et al., Somatic Cell Genet., 3:231-236, 1977.
Gerlach et al., Nature (London), 328:802-805, 1987.
Ghosh and Bachhawat, In: Liver Diseases, Targeted Diagnosis and Therapy Using Specific Receptors and Ligands, Wu and Wu (Eds.), Marcel Dekker, New York, 87-104, 1991.
Gilles et al., Cell, 33:717, 1983.
Gloss et al., EMBO J., 6:3735, 1987.
Gnant et al., Cancer Res., 59(14):3396-403, 1999.
Gnant et al., J. Natl. Cancer Inst., 91(20):1744-1750, 1999.
Godbout et al., Mol. Cell. Biol., 8:1169, 1988.
Goding, In: Monoclonal Antibodies: Principles and Practice, 2d ed., Orlando, Fla., Academic Press, 60-61, 65-66, 71-74, 1986.
Goodbourn and Maniatis, Proc. Natl. Acad. Sci. USA, 85:1447, 1988.
Goodbourn et al., Cell, 45:601, 1986.
Gopal, Mol. Cell. Biol., 5:1188-1190, 1985.
Graham and Prevec Mol. Biotechnol., 3(3):207-220, 1995.
Graham and Van Der Eb, Virology 52:456-467, 1973
Greene et al., Immunology Today, 10:272, 1989
Grishok et al., Science, 287:2494-2497, 2000.
Grosschedl and Baltimore, Cell, 41:885, 1985.
Hacia et al., Nature Genet., 14:441-449, 1996.
Haecker et al., Hum. Gene Ther., 7(15): 1907-1914, 1996.
Han et al., Euro. J Surgical Oncology, 25:194-198, 1999.
Harland and Weintraub, J. Cell Biol., 101:1094-1099, 1985.
Harlow and Lane, In: Antibodies: A Laboratory Manual, Cold Spring Harbor Laboratories, Cold Spring Harbor, N.Y., 1988.
Haslinger and Karin, Proc. Natl. Acad. Sci. USA, 82:8572, 1985.
Hauber and Cullen, J Virology, 62:673, 1988.
Hayashi et al., Neurosci. Lett., 267(1):37-40, 1999.
He et al., Plant Cell Reports, 14 (2-3):192-196, 1994.
Hen et al., Nature, 321:249, 1986.
Hensel et al., Lymphokine Res., 8:347, 1989.
Hermens and Verhaagen, Prog. Neurobiol., 55(4):399-432, 1998.
Herr and Clarke, Cell, 45:461, 1986.
Hirochika et al., J. Virol., 61:2599, 1987.
Hirsch et al., Mol. Cell. Biol., 10:1959, 1990.
Holbrook et al., Virology, 157:211, 1987.
Holzer et al., Virology, 253(1):107-114, 1999.
Horlick and Benfield, Mol. Cell. Biol., 9:2396, 1989.
Hou and Lin, Plant Physiology, 111: 166, 1996.
Howard et al., Ann. NY Acad. Sci., 880:352-365, 1999.
Huang et al., Cell, 27:245, 1981.
Huard et al., Neuromuscul Disord., 7(5):299-313, 1997.
Hug et al., Mol. Cell. Biol., 8:3065, 1988.
Hutvagner et al., Science, 293:834-838, 2001.
Hutvagner and Zamore, Science, 297:2056-2060, 2002.
Hwang et al., Mol. Cell. Biol., 10:585, 1990.
Imagawa et al., Cell, 51:251, 1987.
Imai et al., J. Virol., 72(5):4371-4378, 1998.
Imbra and Karin, Nature, 323:555, 1986.
Imler et al., Mol. Cell. Biol., 7:2558, 1987.
Imperiale and Nevins, Mol. Cell. Biol., 4:875, 1984.
Innis et al., Proc Natl Acad Sci USA, 85(24):9436-9440, 1988.
Irie et al., Antisense Nucleic Acid Drug Dev., 9(4):341-349, 1999.
Jakobovits et al., Mol. Cell. Biol., 8:2555, 1988.
Jameel and Siddiqui, Mol. Cell. Biol., 6:710, 1986.
Jaynes et al., Mol. Cell. Biol., 8:62, 1988.
Johannesson et al., J. Med. Chem., 42(22):4524-4537, 1999.
Johnson et al., In: Biotechnology And Pharmacy, Pezzuto et al., (Eds.), Chapman and Hall, New York, 1993.
Johnson et al., Mol. Cell. Biol., 9:3393, 1989.
Johnston et al., J. Virol., 73(6):4991-5000, 1999.
Joyce, Nature, 338:217-244, 1989.
Kadesch and Berg, Mol. Cell. Biol., 6:2593, 1986.
Kaeppler et al., Plant Cell Reports, 9:415-418, 1990.
Kaneda et al., Science, 243:375-378, 1989.
Karin et al., Mol. Cell. Biol., 7:606, 1987.
Katinka et al., Cell, 20:393, 1980.
Kato et al., J. Biol. Chem., 266(6):3361-3364, 1991.
Kaufman et al., Surv. Opthalmol., 43Suppl 1:S91-97, 1999.
Kauraniemi and Kallioniemi, Endocr. Relat. Cancer, 13:39-49, 2006.
Kauraniemi et al., Am. J. Pathol., 163:1979-1984, 2003.
Kawamoto et al., Mol. Cell. Biol., 8:267, 1988.
Kay, Haemophilia, 4(4):389-392, 1998.
Ketting et al., Cell, 99(2):133-141, 1999.
Kiledjian et al., Mol. Cell. Biol., 8:145, 1988.
Kim and Cech, Proc. Natl. Acad. Sci. USA, 84:8788-8792, 1987.
Klamut et al., Mol. Cell. Biol., 10:193, 1990.
Klimatcheva et al., Front Biosci.,; 4:D481-96, 1999.
Kluck et al., Science, 275:1132-1136, 1997.
Knight and Bass, Science, 2:2, 2001.
Koch et al., Mol. Cell. Biol., 9:303, 1989.
Kohler and Milstein, Eur. J. Immunol., 6:511-519, 1976.
Kohler and Milstein, Nature, 256:495-497, 1975.
Kohut et al., Am. J. Physiol., 275(6 Pt 1):L1089-94, 1998.
Kooby et al., FASEB J, 13(11):1325-1334, 1999.
Komberg, In: DNA Replication, W. H. Freeman and Company, New York, 1992.
Kraus et al., FEBS Lett., 428(3):165-170, 1998.
Kriegler and Botchan, In: Eukaryotic Viral Vectors, Gluzman (Ed.), Cold Spring Harbor: Cold Spring Harbor Laboratory, NY, 1982.
Kriegler and Botchan, Mol. Cell. Biol., 3:325, 1983.
Kriegler et al., Cell, 38:483, 1984.
Kriegler et al., Cell, 53:45, 1988.
Krisky et al., Gene Ther., 5(11):1517-1530, 1998.
Krisky et al., Gene Ther., 5(12):1593-1603, 1998.
Kuhl et al., Cell, 50:1057, 1987.
Kunz et al., Nucl. Acids Res., 17:1121, 1989.
Kwoh et al., Proc. Natl. Acad. Sci. USA, 86(4):1173-1177, 1989.
Kyte and Doolittle, J. Mol. Biol., 157(1):105-132, 1982.
Lachmann and Efstathiou, Clin. Sci. (Colch), 96(6):533-541, 1999.
Lareyre et al., J. Biol. Chem., 274(12):8282-8290, 1999.
Larsen et al., Proc Natl. Acad. Sci. USA., 83:8283, 1986.
Laspia et al., Cell, 59:283, 1989.
Latimer et al., Mol. Cell. Biol., 10:760, 1990.
Latta et al., Mod. Pathol., 15:1318-1325, 2002.
Lazzeri, Methods Mol. Biol., 49:95-106, 1995.
Lee et al., J. Auton. Nerv. Syst., 74(2-3):86-90, 1997.
Lee et al., Korean J. Genet., 11(2):65-72, 1989.
Lee et al., Nature, 294:228, 1981.
Lee et al., Nature, 329(6140):642-645, 1987.
Lee et al., Nucleic Acids Res., 12:4191-206, 1984.
Leibowitz et al., Diabetes, 48(4):745-753, 1999.
Lesch, Biol. Psychiatry, 45(3):247-253, 1999.
Levenson et al., Human Gene Therapy, 9:1233-1236, 1998.
Levine, Cell, 88:323-331, 1997.
Levinson et al., Nature, 295:79, 1982.
Li et al., Science, 275:1943-1947, 1997.
Liang and Pardee, Nature Reviews Cancer, 3:869-876, 2003.
Liang, Biotechniques, 33:338-346, 2002.
Lin et al., Mol. Cell. Biol., 10:850, 1990.
Lundstrom, J. Recept. Signal Transduct. Res., 19(1-4):673-686, 1999.
Luria et al., EMBO J., 6:3307, 1987.
Lusky and Botchan, Proc. Natl. Acad. Sci. USA, 83:3609, 1986.
Lusky et al., Mol. Cell. Biol., 3:1108, 1983.
Macejak and Samow, Nature, 353:90-94, 1991.
Majors and Varmus, Proc. Natl. Acad. Sci. USA, 80:5866, 1983.
Maqani et al., Mol. Cancer. Res., 4:449-455, 2006.
Marienfeld et al., Gene Ther., 6(6): 1101-1113, 1999.
Mastrangelo et al., Cancer Gene Ther., 6(5):409-422 1999.
MeNeall et al., Gene, 76:81, 1989.
Merrifield, Science, 232(4748):341-347 1986.
Michel and Westhof, J. Mol. Biol., 216:585-610, 1990.
Miksicek et al., Cell, 46:203, 1986.
Miller et al., Methods Enzymol., 217:581-599, 1993.
Miyatake et al., Gene Ther., 6(4):564-572, 1999.
Moldawer et al., Shock, 12(2):83-101, 1999.
Monni et al., Proc. Natl. Acad. Sci. USA, 98:5711-5716, 2001.
Montgomery et al., Proc. Natl. Acad. Sci. USA, 95:15502-15507, 1998.
Mordacq and Linzer, Genes and Dev., 3:760, 1989.
Moreau et al., Nucl. Acids Res., 9:6047, 1981.
Moriuchi et al., Cancer Res., 58(24):5731-5737, 1998.
Morrison et al., J. Gen. Virol., 78(Pt 4):873-878, 1997.
Mourelatos et al., Genes Dev., 16(6):720-728, 2002.
Muesing et al., Cell, 48:691, 1987.
Nagata et al., Cancer Cell, 6:117-127, 2004.
Nahta et al., Cancer Res., 65:11118-11128, 2005.
Nahta et al., Nat. Clin. Pract. Oncol., 3:269-280, 2006.
Naldini et al., Proc. Natl. Acad. Sci. USA, 93(21):11382-11388, 1996.
Neumann et al., Proc. Natl. Acad. Sci. USA, 96(16):9345-9350, 1999.
Ng et al., Nuc. Acids Res., 17:601, 1989.
Nicolau and Sene, Biochim. Biophys. Acta, 721:185-190, 1982.
Nicolau et al., Methods Enzymol., 149:157-176, 1987
Nomoto et al., Gene, 236(2):259-271, 1999.
Ohara et al., Proc. Natl. Acad. Sci. USA, 86:5673-5677, 1989.
Omirulleh et al., Plant Mol. Biol., 21(3):415-28, 1993.
Ondek et al., EMBO J., 6:1017, 1987.
Ornitz et al., Mol. Cell. Biol., 7:3466, 1987.
Palmiter et al., Nature, 300:611, 1982.
Parks et al., J. Virol., 71(4):3293-8, 1997.
Pease et al., Proc. Natl. Acad. Sci. USA, 91:5022-5026, 1994.
Pech et al., Mol. Cell. Biol., 9:396, 1989.
Pelletier and Sonenberg, Nature, 334:320-325, 1988.
Peng et al., J. Biol. Chem., 280:13148-13152, 2005.
Perales et al., Proc. Natl. Acad. Sci. USA, 91:4086-4090, 1994.
Perez-Stable and Constantini, Mol. Cell. Biol., 10:1116, 1990.
Petrof, Eur. Respir. J, 11(2):492-497, 1998.
Picard and Schaffner, Nature, 307:83, 1984.
Pignon J et al., Hum. Mutat., 3(2):126-132,1994.
Pinkert et al., Genes and Dev., 1:268, 1987.
Ponta et al., Proc. Natl. Acad. Sci. USA, 82:1020, 1985.
Porton et al., Mol. Cell. Biol., 10:1076, 1990.
Potrykus et al., Mol. Gen. Genet., 199:183-188, 1985.
Potter et al., Proc. Natl. Acad. Sci. USA, 81:7161-7165, 1984.
Queen and Baltimore, Cell, 35:741, 1983.
Quinn et al., Mol. Cell. Biol., 9:4713, 1989.
Rabinovitch et al., Diabetes, 48(6): 1223-1229, 1999.
Reddy et al., J. Virol., 72(2):1394-1402, 1998.
Redondo et al., Science, 247:1225, 1990.
Reinhart et al., Nature, 403:901-906, 2000.
Reinhold-Hurek and Shub, Nature, 357:173-176, 1992.
Reisman and Rotter, Mol. Cell. Biol., 9:3571, 1989.
Remington's Pharmaceutical Sciences, 15^{th} ed., pages 1035-1038 and 1570-1580, Mack Publishing Company, Easton, Pa., 1980.
Remington's Pharmaceutical Sciences 15th Edition, 33:624-652, 1990.
Resendez Jr. et al., Mol. Cell. Biol., 8:4579, 1988.
Rhodes et al., Methods Mol. Biol., 55:121-131, 1995.
Ripe et al., Mol. Cell. Biol., 9:2224, 1989.
Rippe et al., Mol. Cell. Biol., 10:689-695, 1990.
Ritter et al., Clin. Cancer Res., 13(16):4909-4919, 2007.
Rittling et al., Nuc. Acids Res., 17:1619, 1989.
Robbins and Ghivizzani, Pharmacol. Ther., 80(1):35-47, 1998.
Robbins et al., Proc. Natl. Acad. Sci. USA, 95(17):10182-10187 1998.
Robbins et al., Trends Biotechnol., 16(1):35-40, 1998.
Rosen et al., Cell, 41:813, 1988.
Ross et al., Mol. Cell. Proteomics, 3:379-398, 2004.
Rothenberg et al., Proc. Nat'l Acad. Sci. USA 105:12480-4, 2008.
Sakai et al., Genes and Dev., 2:1144, 1988.
Sambrook et al., In:Molecular Cloning: A Laboratory Manual, Vol. 1, Cold Spring Harbor Laboratory Press, Cold Spring Harbor, N.Y., Ch. 7,7,19-17.29, 1989.
Sarver et al., Science, 247:1222-1225, 1990.
Satake et al., J Virology, 62:970, 1988.
Sawai et al., Mol. Genet. Metab., 67(1):36-42, 1999.
Saxena et al., J. Biol. Chem., 278(45):44312-44319, 2003.
Scanlon et al., Proc. Natl. Acad. Sci. USA, 88:10591-10595, 1991.
Schaffner et al., J. Mol. Biol., 201:81, 1988.
Schwartz et al., Cancer Res., 63:2913-2922, 2003.
Schwartze, Z Gesamte Inn. Med., 42:336-339, 1987.
Searle et al., Mol. Cell. Biol., 5:1480, 1985.
Sharp, Genes Dev., 13:139-141, 1999.
Sharp and Marciniak, Cell, 59:229, 1989.
Sharp and Zamore, Science, 287:2431-2433, 2000.
Shaul and Ben-Levy, EMBO J, 6:1913, 1987.
Sherman et al., Mol. Cell. Biol., 9:50, 1989.
Shin et al., Nat. Med., 8:1145-1152, 2002.
Shoemaker et al., Nature Genetics, 14:450-456, 1996.
Sleigh and Lockett, J EMBO, 4:3831, 1985.
Sliwkowski et al., Semin. Oncol., 26:60-70, 1999.
Smith, Arch. Neurol., 55(8):1061-1064, 1998.
Spalholz et al., Cell, 42:183, 1985.
Spandau and Lee, J Virology, 62:427, 1988.
Spandidos and Wilkie, EMBO J, 2:1193, 1983.
Stambolic et al., Mol. Cell, 8:317-325, 2001.
Steck et al., Nat. Genet., 15:356-362, 1997.
Stein et al., J. Biol. Chem., 279:48930-48940, 2004.
Stephens and Hentschel, Biochem. J, 248:1, 1987.
Stephens et al., Nature, 431:525-526, 2004.
Stewart and Young, “Solid Phase Peptide Synthesis”, 2d. ed., Pierce Chemical Co., 1984.
Stewart et al., Gene Ther., 6(3):350-363, 1999.
Stuart et al., Nature, 317:828, 1985.
Sullivan and Peterlin, Mol. Cell. Biol., 7:3315, 1987.
Suzuki et al., Biochem. Biophys. Res. Commun., 252(3):686-690, 1998.
Swartzendruber and Lehman, J. Cell. Physiology, 85:179, 1975.
Tabara et al., Cell, 99(2):123-132, 1999.
Takebe et al., Mol. Cell. Biol., 8:466, 1988.
Tam et al., J. Am. Chem. Soc., 105:6442, 1983.
Tanaka et al., Oncogene, 8:2253-2258, 1993.
Taniura et al., J. Biol. Chem., 274:16242-16248, 1999.
Tavernier et al., Nature, 301:634, 1983.
Taylor and Kingston, Mol. Cell. Biol., 10:165, 1990a.
Taylor and Kingston, Mol. Cell. Biol., 10:176, 1990b.
Taylor and Stark, Oncogene, 20:1803-1815, 2001.
Taylor et al., J. Biol. Chem., 264:15160, 1989.
Thiesen et al., J Virology, 62:614, 1988.
Timiryasova et al., Int. J. Oncol., 14(5):845-854, 1999.
Timiryasova et al., Oncol. Res.; 11(3):133-144, 1999.
Treisman, Cell, 42:889, 1985.
Tronche et al., Mol. Biol. Med., 7:173, 1990.
Trudel and Constantini, Genes and Dev., 6:954, 1987.
Tsukada et al., Plant Cell Physiol., 30(4)599-604, 1989.
Tsumaki et al., J. Biol. Chem., 273(36):22861-22864, 1998.
Tur-Kaspa et al., Mol. Cell. Biol., 6:716-718, 1986.
Tyndell et al., Nuc. Acids. Res., 9:6231, 1981.
Vanderkwaak et al., Gynecol. Oncol., 74(2):227-234, 1999.
Vannice and Levinson, J Virology, 62:1305, 1988.
Varis et al., Cancer Res., 62:2625-2629, 2002.
Varis et al., Int. J. Cancer, 109:548-553, 2004.
Vasseur et al., Proc Natl. Acad. Sci. USA, 77:1068, 1980.
Vogelstein et al., Nature, 408(6810):307-310, 2000.
Vogelstein, Nature, 348(6303):681-682, 1990.
Vousden and Lu, Nat. Rev. Cancer, 2:594-604, 2002.
Vousden and Prives, Cell, 120:7-10, 2005.
Wagner et al., Science, 260:1510-1513, 1990.
Walker et al., Nucleic Acids Res., 20(7):1691-1696, 1992.
Wang and Calame, Cell, 47:241, 1986.
Wang et al., Gynecol. Oncol., 71(2):278-287, 1998.
Weber et al., Cell, 36:983, 1984.
Weihl et al., Neurosurgery, 44(2):239-252, 1999.
Weinberg et al., Biochemistry, 28:8263-8269, 1989.
Weinberger et al., Mol. Cell. Biol., 8:988, 1984.
Weinstein & Joe, Nat. Clin. Pract. Oncol. 3:448-57, 2006.
Weinstein & Joe, Cancer Res. 68:3077-80, 2008.
Weisshoff et al., Eur. J. Biochem., 259(3):776-788, 1999.
White et al., J. Virol., 73(4):2832-28340, 1999.
Wilson, J. Clin. Invest., 98(11):2435, 1996.
Wincott et al., Nucleic Acids Res., 23(14):2677-2684, 1995.
Winoto and Baltimore, Cell, 59:649, 1989.
Wong et al., Gene, 10:87-94, 1980.
Wu and Wallace, Genomics, 4:560-569, 1989.
Wu and Wu, J. Biol. Chem., 262:4429-4432, 1987.
Wu and Wu, Adv. Drug Delivery Rev., 12:159-167, 1993.
Wu et al., Biochem. Biophys. Res. Commun., 233(1):221-226, 1997.
Wu, Chung Hua Min Kuo Hsiao Erh Ko I Hsueh Hui Tsa Chih, 39(5):297-300, 1998.
Xu et al., Curr. Biol., 13(9):790-795, 2003.
Yakes et al., Cancer Res., 62:4132-41, 2002.
Yamada et al., Proc. Natl. Acad. Sci. USA, 96(7):4078-4083, 1999.
Yang and Liang, Mol. Biotechnol., 3:197-208, 2004.
Yang et al., Science, 275:1129-1132, 1997.
Yeung et al., Gene Ther., 6(9):1536-1544, 1999.
Yoon et al., J. Gastrointest. Surg., 3(1):34-48, 1999.
Yu and Zhang, Biochem. Biophys. Res. Commun., 331:851-858, 2005.
Yu et al., Proc. Natl. Acad. Sci. USA, 100:1931-1936, 2003.
Yu et al., Proc. Natl. Acad. Sci. USA, 96:14517-14522, 1999.
Yutzey et al., Mol. Cell. Biol., 9:1397, 1989.
Zeng et al., Cancer Res., 62(13):3630-3635, 2002.
Zhao-Emonet et al., Biochim. Biophys. Acta, 1442(2-3):109-119,1998.
Zheng et al., J. Gen. Virol., 80(Pt 7):1735-1742, 1999.
Zhou et al., Exp. Hematol, 21:928-933, 1993.
Zufferey et al., Nat. Biotechnol., 15(9):871-875, 1997.

What is claimed is:

1. A method of improving the effect of an anti-HER2/Neu therapy in a subject comprising administering to said subject an inhibitor of t-DARPP activity.

2. The method of claim 1, wherein said inhibitor of t-DARPP activity inhibits t-DARPP mRNA synthesis or stability, or t-DARPP protein translation.

3. The method of claim 2, wherein said inhibitor is antisense nucleic acid, an siRNA, or an shRNA.

4. The method of claim 1, wherein said inhibitor of t-DARPP activity inhibits t-DARPP gene product function.

5. The method of claim 4, wherein said inhibitor of t-DARPP gene product function is an antibody, a peptide or a small molecule that binds to t-DARPP.

6. The method of claim 1, wherein said anti-HER2/Neu therapy is anti-HER2/Neu antibody administration.

7. The method of claim 1, wherein said anti-HER2/Neu therapy comprises a reduced dose as compared to an effective dose for said anti-HER2/Neu therapy provided in the absence of said inhibitor of t-DARPP activity.

8. The method of claim 1, wherein said inhibitor of t-DARPP activity is administered to said subject more than once.

9. The method of claim 1, wherein said inhibitor of t-DARPP gene product function is delivered by an expression vector encoding said inhibitor.

10. A method of inhibiting trastuzumab resistance in a subject comprising administering to said subject an inhibitor of t-DARPP activity.

11. A method of inducing apoptosis in a cancer cell the overexpresses ERBB2 comprising contacting said cell with an anti-HER2/Neu agent and an inhibitor of t-DARPP activity.

12. A method of treating cancer in a subject comprising administering to said subject an anti-HER2/Neu therapy and an inhibitor of t-DARPP activity.

13. The method of claim 12, wherein said inhibitor of t-DARPP activity is administered prior to said HER2-Neu therapy.

14. The method of claim 12, wherein said inhibitor of t-DARPP activity is administered during or after said HER2-Neu therapy.

15. The method of claim 12, wherein said cancer is breast cancer.

16. The method of claim 12, wherein said cancer is a Herceptin-resistant breast cancer or a recurrent cancer.

17. The method of claim 12, wherein said inhibitor of t-DARPP activity inhibits t-DARPP mRNA synthesis or stability, or t-DARPP protein translation.

18. The method of claim 17, wherein said inhibitor is antisense nucleic acid, an siRNA, or an shRNA.

19. The method of claim 12, wherein said inhibitor of t-DARPP activity inhibits function of t-DARPP gene product.

20. The method of claim 19, wherein said inhibitor of t-DARPP gene product function is an antibody, a peptide or a small molecule that binds to t-DARPP.

21. The method of claim 12, wherein said anti-HER2/Neu therapy is anti-HER2/Neu antibody administration.

22. The method of claim 12, further comprising altering a dose of either anti-HER2/Neu therapy or the inhibitor of t-DARPP activity based on a clinical response of said patient.

23. The method of claim 12, further comprising assessing t-DARPP protein level or t-DARPP gene amplification in cancer cells of said subject.

24. The method of claim 12, wherein said inhibitor of t-DARPP gene product function is delivered by an expression vector encoding said inhibitor.

25. The method of claim 12, wherein either or both of said anti-HER2Neu therapy and said inhibitor of t-DARPP activity is administered to said subject more than once.

26. A method of inhibiting a cancer cell in a subject, wherein said cancer cell expresses elevated t-DARPP activity as compared to a comparable non-cancer cell, comprising administering to said subject an inhibitor of t-DARPP activity.

27. The method of claim 26, wherein said inhibitor of t-DARPP activity inhibits t-DARPP mRNA synthesis or stability, or t-DARPP protein translation.

28. The method of claim 27, wherein said inhibitor is antisense nucleic acid, an siRNA, or an shRNA.

29. The method of claim 26, wherein said inhibitor of t-DARPP activity inhibits t-DARPP gene product function.

30. The method of claim 29, wherein said inhibitor of t-DARPP gene product function is an antibody, a peptide or a small molecule that binds to t-DARPP.

31. The method of claim 26, wherein said inhibitor of t-DARPP activity is administered to said subject more than once.

32. The method of claim 26, wherein said inhibitor of t-DARPP gene product function is delivered by an expression vector encoding said inhibitor.

33. The method of claim 26, wherein said cancer cell is a gastric carcinoma cell, an esophageal carcinoma cell, or a breast cancer cell.


SEQUENCE LISTING (2 sequences)

SEQ ID NO: 1 - DNA, 1502 bp, Homo sapiens
  CDS (237)..(743)
ttttcatttctcacaaggactgggtgaagagttctgcagccttacagagactggaaaagaagcccaaaccaaggcccccagagaggtcccccaggcccctttgggtccctgagcctcagctggaggtggggggtgcctgcagtgcgctggctcagtctccttctgaaaagctggatccagcttgtttgaagcccttgagctgatcttagatccggcgcaggagaccaacgcctgccatgctgttccggctctcagagcactcctcaccagaggaggaagcctccccccaccagagagcctcaggagaggggcaccatctcaagtcgaagagacccaacccctgtgcctacacaccaccttcgctgaaagctgtgcagcgcattgctgagtctcacctgcagtctatcagcaatttgaatgagaaccaggcctcagaggaggaggatgagctgggggagcttcgggagctgggttatccaagagaggaagatgaggaggaagaggaggatgatgaagaagaggaagaagaagaggacagccaggctgaagtcctgaaggtcatcaggcagtctgctgggcaaaagacaacctgtggccagggtctggaagggccctgggagcgcccaccccctctggatgagtccgagagagatggaggctctgaggaccaagtggaagacccagcactaagtgagcctggggaggaacctcagcgcccttccccctctgagcctggcacataggcacccagcctgcatctcccaggaggaagtggaggggacatcgctgttccccagaaacccactctatcctcaccctgttttgtgctcttcccctcgcctgctagggctgcggcttctgacttctagaagactaaggctggtctgtgtttgcttgtttgcccacctttggctgatacccagagaacctgggcacttgctgcctgatgcccacccctgccagtcattcctccattcacccagcgggaggtgggatgtgagacagcccacattggaaaatccagaaaaccgggaacagggatttgcccttcacaattctactccccagatcctctcccctggacacaggagacccacagggcaggaccctaagatctggggaaaggaggtcctgagaaccttgaggtacccttagatccttttctacccactttcctatggaggattccaagtcaccacttctctcaccggcttctaccagggtccaggactaaggcgtttttctccatagcctcaacattttgggaatcttcccttaatcacccttgctcctcctgggtgcctggaagatggactggcagagacctctttgttgcgttttgtgctttgatgccaggaatgccgcctagtttatgtccccggtggggcacacagcggggggcgccaggttttccttgtcccccagctgctctgcccctttccccttcttccctgactccaggcctgaacccctcccgtgctgtaataaatctttgtaaataa

SEQ ID NO: 2 - protein, 168 aa, Homo sapiens
MLFRLSEHSSPEEEASPHQRASGEGHHLKSKRPNPCAYTPPSLKAVQRIAESHLQSISNLNENQASEEEDELGELRELGYPREEDEEEEEDDEEEEEEEDSQAEVLKVIRQSAGQKTTCGQGLEGPWERPPPLDESERDGGSEDQVEDPALSEPGEEPQRPSPSEPGT